SEQUENCE LISTING (237 sequences)

SEQ ID NO: 1 - protein, 876 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVHFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYQMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGQLTDEQVIENAGEFFRDQGRQVYLSRPPACPQGLYELMLRCWSRESEQRPPFSQLHRFLAEDALNTV

SEQ ID NO: 2 - protein, 396 aa, Homo sapiens
DMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVHFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPT

SEQ ID NO: 3 - DNA, 405 bp, Mus musculus
atgagagtgctgattcttttgtgcctgttcacagcctttcctggtatcctgtctgatgtacatcttcaggagtcaggacctgacctggtgaaaccttctcagtcactttcactcacctgcactgtcactgactactccatcaccagtggttatggctggcactggatccggcagtttccaggaaacaaactggaatggatgggctacatacactacggtggtagcactaagtacaattcatctctcaaaagtcgcatctctatcactcgagacacatccaagaaccagttcttcctgcaattgaaatctgtgactactgatgacacaggcacatattactgtgcaacctctggtaactacggatttgcttactggggccaagggactctggtcactgtctctgca

SEQ ID NO: 4 - protein, 135 aa, Mus musculus
MRVLILLCLFTAFPGILSDVHLQESGPDLVKPSQSLSLTCTVTDYSITSGYGWHWIRQFPGNKLEWMGYIHYGGSTKYNSSLKSRISITRDTSKNQFFLQLKSVTTDDTGTYYCATSGNYGFAYWGQGTLVTVSA

SEQ ID NO: 5 - DNA, 33 bp, Mus musculus
gactactccatcaccagtggttatggctggcac

SEQ ID NO: 6 - protein, 11 aa, Mus musculus
DYSITSGYGWH

SEQ ID NO: 7 - DNA, 48 bp, Mus musculus
tacatacactacggtggtagcactaagtacaattcatctctcaaaagt

SEQ ID NO: 8 - protein, 16 aa, Mus musculus
YIHYGGSTKYNSSLKS

SEQ ID NO: 9 - DNA, 24 bp, Mus musculus
tctggtaactacggatttgcttac

SEQ ID NO: 10 - protein, 8 aa, Mus musculus
SGNYGFAY

SEQ ID NO: 11 - DNA, 396 bp, Mus musculus
atggagacagacacactcctgctatgggtgctgctgctctgggttccaggttccacaggtgacattgtgctgacccaatctccaacttctttggctgtgtctcttgggcagagggccaccatgtcctgcagagccagtgaaagtgttgatgattatggcactagttttatgcactggtaccagcagaaaccaggacagccacccaaactcctcatctatcgtgcatccaacctagaatctgggatccctgccaggttcagtggcagtgggtctagggcagacttcaccctcagcattaatcctgtggagcctgatgatgttgcaacctattactgtcagcaaagtaatgaggatccattcacgttcggctcggggacaaagttggaaataaaacgg

SEQ ID NO: 12 - protein, 132 aa, Mus musculus
METDTLLLWVLLLWVPGSTGDIVLTQSPTSLAVSLGQRATMSCRASESVDDYGTSFMHWYQQKPGQPPKLLIYRASNLESGIPARFSGSGSRADFTLSINPVEPDDVATYYCQQSNEDPFTFGSGTKLEIKR

SEQ ID NO: 13 - DNA, 45 bp, Mus musculus
agagccagtgaaagtgttgatgattatggcactagttttatgcac

SEQ ID NO: 14 - protein, 15 aa, Mus musculus
RASESVDDYGTSFMH

SEQ ID NO: 15 - DNA, 21 bp, Mus musculus
cgtgcatccaacctagaatct

SEQ ID NO: 16 - protein, 7 aa, Mus musculus
RASNLES

SEQ ID NO: 17 - DNA, 27 bp, Mus musculus
cagcaaagtaatgaggatccattcacg

SEQ ID NO: 18 - protein, 9 aa, Mus musculus
QQSNEDPFT

SEQ ID NO: 19 - DNA, 414 bp, Mus musculus
atgaacttagggctcagcttcattttccttgcccttattttaaaaggtgtccagtgtgaggtgcagctggtggagtctgggggaggcttagtgcagcctggagggtccctgaaattctcctgtgcagcctctggattcactttcagtagctatggcatgtcttgggttcgccagactccagacaagaggctggagttggtcgcaattattaatagtcatggttttagcacctattatcctgacagtgtgaagggccgattcaccatctccagagacaatgccaagaacaccctgtacctgcaaatgagcagtcttaagtctgaggacacagccatatattactgtgcaagaggggggtatggtaactacgggatggactactggggtcaaggaacctcagtcaccgtctcctca

SEQ ID NO: 20 - protein, 138 aa, Mus musculus
MNLGLSFIFLALILKGVQCEVQLVESGGGLVQPGGSLKFSCAASGFTFSSYGMSWVRQTPDKRLELVAIINSHGFSTYYPDSVKGRFTISRDNAKNTLYLQMSSLKSEDTAIYYCARGGYGNYGMDYWGQGTSVTVSS

SEQ ID NO: 21 - DNA, 30 bp, Mus musculus
ggattcactttcagtagctatggcatgtct

SEQ ID NO: 22 - protein, 10 aa, Mus musculus
GFTFSSYGMS

SEQ ID NO: 23 - DNA, 51 bp, Mus musculus
attattaatagtcatggttttagcacctattatcctgacagtgtgaagggc

SEQ ID NO: 24 - protein, 17 aa, Mus musculus
IINSHGFSTYYPDSVKG

SEQ ID NO: 25 - DNA, 30 bp, Mus musculus
ggggggtatggtaactacgggatggactac

SEQ ID NO: 26 - protein, 10 aa, Mus musculus
GGYGNYGMDY

SEQ ID NO: 27 - DNA, 396 bp, Mus musculus
atggagacagacacactcctgctatgggtgctgctgctctgggttccaggttccacaggtgacattgtgctgacccaatctccagcttctttggctgtgtctctagggcagagggccaccatatcctgcagagccagtgaaagtgttgatagttatggcaatagttttatgcactggtaccagcagaaagttggacagccacccaaactcctcatctttcgtgcatccaatatagaatctgggatccctgccaggttcagtggcagtgggtctaggacagacttcaccctcaccattaattctgtggaggctgatgatgttgcaacctattactgtcagcaaagtaatgaggctccgtacacgttcggaggggggaccaagctggaaataaaacgg

SEQ ID NO: 28 - protein, 132 aa, Mus musculus
METDTLLLWVLLLWVPGSTGDIVLTQSPASLAVSLGQRATISCRASESVDSYGNSFMHWYQQKVGQPPKLLIFRASNIESGIPARFSGSGSRTDFTLTINSVEADDVATYYCQQSNEAPYTFGGGTKLEIKR

SEQ ID NO: 29 - DNA, 45 bp, Mus musculus
agagccagtgaaagtgttgatagttatggcaatagttttatgcac

SEQ ID NO: 30 - protein, 15 aa, Mus musculus
RASESVDSYGNSFMH

SEQ ID NO: 31 - DNA, 21 bp, Mus musculus
cgtgcatccaatatagaatct

SEQ ID NO: 32 - protein, 7 aa, Mus musculus
RASNIES

SEQ ID NO: 33 - DNA, 27 bp, Mus musculus
cagcaaagtaatgaggctccgtacacg

SEQ ID NO: 34 - protein, 9 aa, Mus musculus
QQSNEAPYT

SEQ ID NO: 35 - DNA, 414 bp, Mus musculus
atggaaaggcactggatctttctcttcctgttttcagtaactgcaggtgtccactcccaggtccagcttcaccagtctgggtctgaactggcaaaacctggggcctcagtgaagatgtcctgcaaggcttctggctacatctttactacctactggatacactggttaaaacagaggcctggacagggtctggaatggattggattcattaatcctgacactgattatactgaatacaatcagaagttcaaggacaaggccacattgactgcagacaaatcctccaacacagcctacgtgcaactgagcagcctgacatctgaggactctgcagtctattactgtgcgagaggaaactatggttacgacggtcttgtttactggggcctagggactctggtcactgtctctgca

SEQ ID NO: 36 - protein, 138 aa, Mus musculus
MERHWIFLFLFSVTAGVHSQVQLHQSGSELAKPGASVKMSCKASGYIFTTYWIHWLKQRPGQGLEWIGFINPDTDYTEYNQKFKDKATLTADKSSNTAYVQLSSLTSEDSAVYYCARGNYGYDGLVYWGLGTLVTVSA

SEQ ID NO: 37 - DNA, 30 bp, Mus musculus
ggctacatctttactacctactggatacac

SEQ ID NO: 38 - protein, 10 aa, Mus musculus
GYIFTTYWIH

SEQ ID NO: 39 - DNA, 51 bp, Mus musculus
ttcattaatcctgacactgattatactgaatacaatcagaagttcaaggac

SEQ ID NO: 40 - protein, 17 aa, Mus musculus
FINPDTDYTEYNQKFKD

SEQ ID NO: 41 - DNA, 30 bp, Mus musculus
ggaaactatggttacgacggtcttgtttac

SEQ ID NO: 42 - protein, 10 aa, Mus musculus
GNYGYDGLVY

SEQ ID NO: 43 - DNA, 381 bp, Mus musculus
atgaagtcacagacccaggtcttcgtatttctactgctctgtgtgtctggtgctcatgggagtattgtgatgacccagactcccaaattcctgcttgtatcagcaggagacagggttaccataacctgcaaggccagtcagagtgtgagtactgatgtagcttggtaccaacagaagccagggcagtctcctaaactgctgatatactatgcatccaatcgctacactggagtccctgatcgcttcactggcagtggatatgggacggatttcactttcaccatcagcactgtgcaggctgaagacctggcagtttatttctgtcagcaggattataactcgtacacgttcggaggggggaccaagctggaaataaaacgg

SEQ ID NO: 44 - protein, 127 aa, Mus musculus
MKSQTQVFVFLLLCVSGAHGSIVMTQTPKFLLVSAGDRVTITCKASQSVSTDVAWYQQKPGQSPKLLIYYASNRYTGVPDRFTGSGYGTDFTFTISTVQAEDLAVYFCQQDYNSYTFGGGTKLEIKR

SEQ ID NO: 45 - DNA, 33 bp, Mus musculus
aaggccagtcagagtgtgagtactgatgtagct

SEQ ID NO: 46 - protein, 11 aa, Mus musculus
KASQSVSTDVA

SEQ ID NO: 47 - DNA, 21 bp, Mus musculus
tatgcatccaatcgctacact

SEQ ID NO: 48 - protein, 7 aa, Mus musculus
YASNRYT

SEQ ID NO: 49 - DNA, 24 bp, Mus musculus
cagcaggattataactcgtacacg

SEQ ID NO: 50 - protein, 8 aa, Mus musculus
QQDYNSYT

SEQ ID NO: 51 - DNA, 420 bp, Mus musculus
atggaatggagctggatctttctcttcctcctgtcaggaacttcaggtgtccactctgagatccagctgcagcagtctggacctgagctggtgaaccctggggcttcagtgaaggtatcctgcaaggcttctggttactcattcactgactacaacatgtactgggtgaagcagagccatggaaagagccttgagtggattggatatattgatccttacaatggtggtactagctataatcagaagttcaagggcaaggccacattgactgttgacaagtcctccagcacagccttcatgcatctcaacagcctgacatctgaggactctgcagtctattactgtgcaagagagtactacggtagtagactttggtacttcgatgtctggggcgcagggaccgcggtcaccgtctcctca

SEQ ID NO: 52 - protein, 140 aa, Mus musculus
MEWSWIFLFLLSGTSGVHSEIQLQQSGPELVNPGASVKVSCKASGYSFTDYNMYWVKQSHGKSLEWIGYIDPYNGGTSYNQKFKGKATLTVDKSSSTAFMHLNSLTSEDSAVYYCAREYYGSRLWYFDVWGAGTAVTVSS

SEQ ID NO: 53 - DNA, 30 bp, Mus musculus
ggttactcattcactgactacaacatgtac

SEQ ID NO: 54 - protein, 10 aa, Mus musculus
GYSFTDYNMY

SEQ ID NO: 55 - DNA, 51 bp, Mus musculus
tatattgatccttacaatggtggtactagctataatcagaagttcaagggc

SEQ ID NO: 56 - protein, 17 aa, Mus musculus
YIDPYNGGTSYNQKFKG

SEQ ID NO: 57 - DNA, 36 bp, Mus musculus
gagtactacggtagtagactttggtacttcgatgtc

SEQ ID NO: 58 - protein, 12 aa, Mus musculus
EYYGSRLWYFDV

SEQ ID NO: 59 - DNA, 402 bp, Mus musculus
atggaatcacagacccaggtcctcatgtttcttctgctctgggtatctggtgcctgtgtagacattgtgatgacacagtctccatcctccctggctatgtcagtaggacagaaggtcactatgagctgcaagtccagtcagagtcttttaaataggaacaatcaaaagaactatttggcctggtaccagcagaaaccaggacagtctcctaaacttctggtatactttgcatccactagggaatctggggtccctgatcgcttcataggcagtggatctgggacagatttcactcttaccctcagcagtgtgcgggctgaagacctggcagaaaacttctgtcagcaacattatagcactccgttcacgttcggctcggggacaaggttggaaataaaacgg

SEQ ID NO: 60 - protein, 134 aa, Mus musculus
MESQTQVLMFLLLWVSGACVDIVMTQSPSSLAMSVGQKVTMSCKSSQSLLNRNNQKNYLAWYQQKPGQSPKLLVYFASTRESGVPDRFIGSGSGTDFTLTLSSVRAEDLAENFCQQHYSTPFTFGSGTRLEIKR

SEQ ID NO: 61 - DNA, 51 bp, Mus musculus
aagtccagtcagagtcttttaaataggaacaatcaaaagaactatttggcc

SEQ ID NO: 62 - protein, 17 aa, Mus musculus
KSSQSLLNRNNQKNYLA

SEQ ID NO: 63 - DNA, 21 bp, Mus musculus
tttgcatccactagggaatct

SEQ ID NO: 64 - protein, 7 aa, Mus musculus
FASTRES

SEQ ID NO: 65 - DNA, 27 bp, Mus musculus
cagcaacattatagcactccgttcacg

SEQ ID NO: 66 - protein, 9 aa, Mus musculus
QQHYSTPFT

SEQ ID NO: 67 - DNA, 423 bp, Mus musculus
atgaacttcgggctcagcttgattttccttgtccttgttttaaaaggtgtccagtgtgaagtgatgctggtggagtctgggggaggcttagtgaagcctggagggtccctgaaactctcctgtgcagcctctggattcactttcagtaactatgccatgtcttgggttcgccagactccggagaagaggctggagtgggtcgcaaaaactagtagtggtgatacttacacctactatccagacagtgtgaaggggcgattcaccatctccagggacaatgccaagaacaccctgtacctgcaaatgaccagtctgaggtctgaggacacggccatttattactgtgcaagacaagactactacggtattaccccttggttcttcgatgtctggggcgcagggaccacggtcaccgtctcctca

SEQ ID NO: 68 - protein, 141 aa, Mus musculus
MNFGLSLIFLVLVLKGVQCEVMLVESGGGLVKPGGSLKLSCAASGFTFSNYAMSWVRQTPEKRLEWVAKTSSGDTYTYYPDSVKGRFTISRDNAKNTLYLQMTSLRSEDTAIYYCARQDYYGITPWFFDVWGAGTTVTVSS

SEQ ID NO: 69 - DNA, 30 bp, Mus musculus
ggattcactttcagtaactatgccatgtct

SEQ ID NO: 70 - protein, 10 aa, Mus musculus
GFTFSNYAMS

SEQ ID NO: 71 - DNA, 51 bp, Mus musculus
aaaactagtagtggtgatacttacacctactatccagacagtgtgaagggg

SEQ ID NO: 72 - protein, 17 aa, Mus musculus
KTSSGDTYTYYPDSVKG

SEQ ID NO: 73 - DNA, 39 bp, Mus musculus
caagactactacggtattaccccttggttcttcgatgtc

SEQ ID NO: 74 - protein, 13 aa, Mus musculus
QDYYGITPWFFDV

SEQ ID NO: 75 - DNA, 396 bp, Mus musculus
atggattttctggtgcagattttcagcttcttgctaatcagtgcctcagttgcaatgtccagaggagaaaatgtgctcacccagtctccagcaatcatgtctgcatctccaggggaaaaggtcaccatgacctgcagggccagctcaagtgtaagttccatttacttgcactggtaccagcagaagtcaggtgcctcccccaaactctggatttatagcacatccaacttggcttctggagtccctgctcgcttcagtggcagtgggtctgggacctcttactctctcacaatcaccagtgtggaggctgaagatgctgccacttattactgccagcagtacagtggttacccactcatcacgttcggtgctgggaccaagctggagctgaaacgg

SEQ ID NO: 76 - protein, 132 aa, Mus musculus
MDFLVQIFSFLLISASVAMSRGENVLTQSPAIMSASPGEKVTMTCRASSSVSSIYLHWYQQKSGASPKLWIYSTSNLASGVPARFSGSGSGTSYSLTITSVEAEDAATYYCQQYSGYPLITFGAGTKLELKR

SEQ ID NO: 77 - DNA, 36 bp, Mus musculus
agggccagctcaagtgtaagttccatttacttgcac

SEQ ID NO: 78 - protein, 12 aa, Mus musculus
RASSSVSSIYLH

SEQ ID NO: 79 - DNA, 21 bp, Mus musculus
agcacatccaacttggcttct

SEQ ID NO: 80 - protein, 7 aa, Mus musculus
STSNLAS

SEQ ID NO: 81 - DNA, 30 bp, Mus musculus
cagcagtacagtggttacccactcatcacg

SEQ ID NO: 82 - protein, 10 aa, Mus musculus
QQYSGYPLIT

SEQ ID NO: 83 - DNA, 423 bp, Mus musculus
atggattggctgtggaacttgctattcctgatggcagctgcccaaagtgcccaagcacagatccagttggtgcaatctggacctgagctgaagaagtctggagagacagtcaagatctcctgcaaggcttctgggtataccttcacagactatggaatgaactgggtgaagcaggctccaggaaagggtttaaagtggatgggctggatcaacacctacactggaaagtcaacatatgctgatgacttcaagggacggtttgccttctctttggaaacctctgccagcactacctatttgcagatcaccaacctcaaaaatgaggacatggctacatatttctgtgcgagatgggcggattacttcggtagtagctacctgtttgcttcctggggccaagggactctggtcactgtctctgca

SEQ ID NO: 84 - protein, 141 aa, Mus musculus
MDWLWNLLFLMAAAQSAQAQIQLVQSGPELKKSGETVKISCKASGYTFTDYGMNWVKQAPGKGLKWMGWINTYTGKSTYADDFKGRFAFSLETSASTTYLQITNLKNEDMATYFCARWADYFGSSYLFASWGQGTLVTVSA

SEQ ID NO: 85 - DNA, 39 bp, Mus musculus
aaggcttctgggtataccttcacagactatggaatgaac

SEQ ID NO: 86 - protein, 13 aa, Mus musculus
KASGYTFTDYGMN

SEQ ID NO: 87 - DNA, 51 bp, Mus musculus
tggatcaacacctacactggaaagtcaacatatgctgatgacttcaaggga

SEQ ID NO: 88 - protein, 17 aa, Mus musculus
WINTYTGKSTYADDFKG

SEQ ID NO: 89 - DNA, 39 bp, Mus musculus
tgggcggattacttcggtagtagctacctgtttgcttcc

SEQ ID NO: 90 - protein, 13 aa, Mus musculus
WADYFGSSYLFAS

SEQ ID NO: 91 - DNA, 396 bp, Mus musculus
atgaagttgcctgttaggctgttggtgctgatgttctggattcctgtttccagcagtgatgttttgatgacccaaactccactctccctgcctgtcagtcttggagatcaagcctccatctcttgcagatctagtcagagcattgtacatagtgatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggaatttattactgctttcaaggttcacatgttccgtggacgttcggtggaggcaccaagctggaaatcaaacgg

SEQ ID NO: 92 - protein, 132 aa, Mus musculus
MKLPVRLLVLMFWIPVSSSDVLMTQTPLSLPVSLGDQASISCRSSQSIVHSDGNTYLEWYLQKPGQSPKLLIYKVSNRFSGVPDRFSGSGSGTDFTLKISRVEAEDLGIYYCFQGSHVPWTFGGGTKLEIKR

SEQ ID NO: 93 - DNA, 48 bp, Mus musculus
agatctagtcagagcattgtacatagtgatggaaacacctatttagaa

SEQ ID NO: 94 - protein, 16 aa, Mus musculus
RSSQSIVHSDGNTYLE

SEQ ID NO: 95 - DNA, 21 bp, Mus musculus
aaagtttccaaccgattttct

SEQ ID NO: 96 - protein, 7 aa, Mus musculus
KVSNRFS

SEQ ID NO: 97 - DNA, 27 bp, Mus musculus
tttcaaggttcacatgttccgtggacg

SEQ ID NO: 98 - protein, 9 aa, Mus musculus
FQGSHVPWT

SEQ ID NO: 99 - DNA, 420 bp, Mus musculus
atggctgtcttggggctgctcttctgcctggtgacattcccaagctgtgtcctatcccaggtgcagctgaagcagtcaggacctggcctagtgcagccctcacagagcctgtccatcacctgcacagtctctggtttctcatttactggctatggtatacactgggttcgccagtctccaggaaagggtctggagtggctcggagtgatttggagtggtggaatcacagactataatgcagctttcatatccagactgagcatcagcatggacagttccaagggccaagttttctttaaaatgaacagtctgcaagctaatgacacagccatatattactgtgccagaagaagtactacggtagtggccgactatgctatggactactggggtcaaggaacctcagtcaccgtctcctca

SEQ ID NO: 100 - protein, 140 aa, Mus musculus
MAVLGLLFCLVTFPSCVLSQVQLKQSGPGLVQPSQSLSITCTVSGFSFTGYGIHWVRQSPGKGLEWLGVIWSGGITDYNAAFISRLSISMDSSKGQVFFKMNSLQANDTAIYYCARRSTTVVADYAMDYWGQGTSVTVSS

SEQ ID NO: 101 - DNA, 30 bp, Mus musculus
ggtttctcatttactggctatggtatacac

SEQ ID NO: 102 - protein, 10 aa, Mus musculus
GFSFTGYGIH

SEQ ID NO: 103 - DNA, 48 bp, Mus musculus
gtgatttggagtggtggaatcacagactataatgcagctttcatatcc

SEQ ID NO: 104 - protein, 16 aa, Mus musculus
VIWSGGITDYNAAFIS

SEQ ID NO: 105 - DNA, 39 bp, Mus musculus
agaagtactacggtagtggccgactatgctatggactac

SEQ ID NO: 106 - protein, 13 aa, Mus musculus
RSTTVVADYAMDY

SEQ ID NO: 107 - DNA, 384 bp, Mus musculus
atgaagtcacagacccaggtcttcgtatttctactgctctgtgtgtctggtgctcatgggagtattgtgatgacccagactcccaagttcctgcttgtatcagcaggagacagggttaccataacctgcaaggccagtcagagtgtgagtaatgatgtagcttggtaccaacagaagccagggcagtctcctaaactgctgatatactatgcatccaatcgctacactggagtccctgatcgcttcactggcagtggatatgggacggatttcactttcaccatcaacactgtgcaggctgaagacctggcagtttatttctgtcaacaggattatacctctcctctcacgttcggtgctgggaccaagctggagctgaaacgg

SEQ ID NO: 108 - protein, 128 aa, Mus musculus
MKSQTQVFVFLLLCVSGAHGSIVMTQTPKFLLVSAGDRVTITCKASQSVSNDVAWYQQKPGQSPKLLIYYASNRYTGVPDRFTGSGYGTDFTFTINTVQAEDLAVYFCQQDYTSPLTFGAGTKLELKR

SEQ ID NO: 109 - DNA, 33 bp, Mus musculus
aaggccagtcagagtgtgagtaatgatgtagct

SEQ ID NO: 110 - protein, 11 aa, Mus musculus
KASQSVSNDVA

SEQ ID NO: 111 - DNA, 21 bp, Mus musculus
tatgcatccaatcgctacact

SEQ ID NO: 112 - protein, 7 aa, Mus musculus
YASNRYT

SEQ ID NO: 113 - DNA, 27 bp, Mus musculus
caacaggattatacctctcctctcacg

SEQ ID NO: 114 - protein, 9 aa, Mus musculus
QQDYTSPLT

SEQ ID NO: 115 - DNA, 420 bp, Mus musculus
atggctgtcttggggctgctcttctgcctggtgacattcccaagctgtgtcctatcccaggtgctgctgaggcagtcaggacctggcctagtgcagccctcacagagcctgtccatcacctgcacagtctctggtttctcattaactacctatggtgtacactgggttcgccagtctccaggaaagggtctggagtggctgggagtgatatggagtggtggaagcacagactataatgcagctttcatatccagactgaccatcagcaaggacaattccaagagccaagttttctttaaaatgaacagtctgcaagctaatgacacagccatatattactgtgccagatattactccggtagtagcctttactttgctatggactactggggtcaaggaacctcagtcaccgtctcctca

SEQ ID NO: 116 - protein, 140 aa, Mus musculus
MAVLGLLFCLVTFPSCVLSQVLLRQSGPGLVQPSQSLSITCTVSGFSLTTYGVHWVRQSPGKGLEWLGVIWSGGSTDYNAAFISRLTISKDNSKSQVFFKMNSLQANDTAIYYCARYYSGSSLYFAMDYWGQGTSVTVSS

SEQ ID NO: 117 - DNA, 30 bp, Mus musculus
ggtttctcattaactacctatggtgtacac

SEQ ID NO: 118 - protein, 10 aa, Mus musculus
GFSLTTYGVH

SEQ ID NO: 119 - DNA, 48 bp, Mus musculus
gtgatatggagtggtggaagcacagactataatgcagctttcatatcc

SEQ ID NO: 120 - protein, 16 aa, Mus musculus
VIWSGGSTDYNAAFIS

SEQ ID NO: 121 - DNA, 39 bp, Mus musculus
tattactccggtagtagcctttactttgctatggactac

SEQ ID NO: 122 - protein, 13 aa, Mus musculus
YYSGSSLYFAMDY

SEQ ID NO: 123 - DNA, 383 bp, Mus musculus
atgaagtcacagacccaggtcttcgtatttctactgcctctgtgtgtctggtgctcatgggaatattgtgatgacccagactcccaaattccctgcttgtatcagcaggagacacggttaccataacctgcaaggccagtcagagtgtgagtaatgatgttgcttggtaccaacagaagccagggcagtctcctaaactgctgatatactatgcatccaatcgctacactggagtccctgatcgcttcactggcagtggatttgggacggatttcactttcaccatcagcactgtgcagactgaagacctggcagtttatttctgtcagcaggattatagctcgctcacgttcggtgctgggaccaagctggagctgaaacgg

SEQ ID NO: 124 - protein, 127 aa, Mus musculus
MKSQTQVFVFLLLCVSGAHGNIVMTQTPKFLLVSAGDTVTITCKASQSVSNDVAWYQQKPGQSPKLLIYYASNRYTGVPDRFTGSGFGTDFTFTISTVQTEDLAVYFCQQDYSSLTFGAGTKLELKR

SEQ ID NO: 125 - DNA, 33 bp, Mus musculus
aaggccagtcagagtgtgagtaatgatgttgct

SEQ ID NO: 126 - protein, 11 aa, Mus musculus
KASQSVSNDVA

SEQ ID NO: 127 - DNA, 21 bp, Mus musculus
tatgcatccaatcgctacact

SEQ ID NO: 128 - protein, 7 aa, Mus musculus
YASNRYT

SEQ ID NO: 129 - DNA, 24 bp, Mus musculus
cagcaggattatagctcgctcacg

SEQ ID NO: 130 - protein, 8 aa, Mus musculus
QQDYSSLT

SEQ ID NO: 131 - DNA, 408 bp, Mus musculus
atggctgtcctggggctgcttctctgcctggtgactttcccaagctgtgtcctgtcccaggtacagttgaaggagtcaggacctggcctggtggcgccctcacagagcctgtccatcacatgcaccgtctcagggttttcattaactagttatcatatacactgggttcgtcagcctccaggaaagggtctggagtggctggtagtgatatggagtgatggaagcacaacctataattcagctctcaaatccagactgagcatcagcaaggacaactccaagagccaagttttcttaaaaatgaacagtctccaaactgatgacacagccatttactactgtgccagaactggtaatttttatgctatggactactggggtcaaggaacctcagtcaccgtctcctca

SEQ ID NO: 132 - protein, 136 aa, Mus musculus
MAVLGLLLCLVTFPSCVLSQVQLKESGPGLVAPSQSLSITCTVSGFSLTSYHIHWVRQPPGKGLEWLVVIWSDGSTTYNSALKSRLSISKDNSKSQVFLKMNSLQTDDTAIYYCARTGNFYAMDYWGQGTSVTVSS

SEQ ID NO: 133 - DNA, 30 bp, Mus musculus
gggttttcattaactagttatcatatacac

SEQ ID NO: 134 - protein, 10 aa, Mus musculus
GFSLTSYHIH

SEQ ID NO: 135 - DNA, 48 bp, Mus musculus
gtgatatggagtgatggaagcacaacctataattcagctctcaaatcc

SEQ ID NO: 136 - protein, 16 aa, Mus musculus
VIWSDGSTTYNSALKS

SEQ ID NO: 137 - DNA, 27 bp, Mus musculus
actggtaatttttatgctatggactac

SEQ ID NO: 138 - protein, 9 aa, Mus musculus
TGNFYAMDY

SEQ ID NO: 139 - DNA, 381 bp, Mus musculus
atgaagtcacagacccaggtcttcgtatttctactgctctgtgtgtctggtgctcatgggagtattgtgatgacccagactcccaaattcctgcttgtatcagcaggagacagggttaccataacctgcaaggccagtcagagtgtgagtaatgatgtagtttggtaccaacagaagccagggcagtctcctaaactgctgatataytatgcatccagtcgctacactggagtccctgatcgcttcactggcagtggatatgggacggatttcactttcaccatcagcactgtgcaggctgaagacctggcagtttatttctgtcagcaggattatagctctccgtacacgttcggaggggggaccaagctggaaataaga

SEQ ID NO: 140 - protein, 127 aa, Mus musculus
MKSQTQVFVFLLLCVSGAHGSIVMTQTPKFLLVSAGDRVTITCKASQSVSNDVVWYQQKPGQSPKLLIYYASSRYTGVPDRFTGSGYGTDFTFTISTVQAEDLAVYFCQQDYSSPYTFGGGTKLEIR

SEQ ID NO: 141 - DNA, 33 bp, Mus musculus
aaggccagtcagagtgtgagtaatgatgtagtt

SEQ ID NO: 142 - protein, 11 aa, Mus musculus
KASQSVSNDVV

SEQ ID NO: 143 - DNA, 21 bp, Mus musculus
tatgcatccagtcgctacact

SEQ ID NO: 144 - protein, 7 aa, Mus musculus
YASSRYT

SEQ ID NO: 145 - DNA, 27 bp, Mus musculus
cagcaggattatagctctccgtacacg

SEQ ID NO: 146 - protein, 9 aa, Mus musculus
QQDYSSPYT

SEQ ID NO: 147 - DNA, 408 bp, Mus musculus
atgaacttcgggttcagcttgattttccttgtccttgttttaaaaggtgtccagtgtgaagtgaacctggtggagtctgggggaggcttagtgaagcctggagggtccctgaaactctcctgtgcagcctctggattcactttcagtacctatgccatgtcttgggttcgccaaactccagagaagaggctggagtgggtcgcatccattagtagtggtggtatcacccactatccagacagtatgaagggccgattcaccgtctccagagataatgccaggaacatcctgtacctgcaaatgcgcagtctgaggtctgaggacacggccaagtattactgtgcaaggggtcgatcttctatgatccccgactactggggtcaaggaacctcagtctccgtctcctca

SEQ ID NO: 148 - protein, 136 aa, Mus musculus
MNFGFSLIFLVLVLKGVQCEVNLVESGGGLVKPGGSLKLSCAASGFTFSTYAMSWVRQTPEKRLEWVASISSGGITHYPDSMKGRFTVSRDNARNILYLQMRSLRSEDTAKYYCARGRSSMIPDYWGQGTSVSVSS

SEQ ID NO: 149 - DNA, 30 bp, Mus musculus
ggattcactttcagtacctatgccatgtct

SEQ ID NO: 150 - protein, 10 aa, Mus musculus
GFTFSTYAMS

SEQ ID NO: 151 - DNA, 48 bp, Mus musculus
tccattagtagtggtggtatcacccactatccagacagtatgaagggc

SEQ ID NO: 152 - protein, 16 aa, Mus musculus
SISSGGITHYPDSMKG

SEQ ID NO: 153 - protein, 27 aa, Mus musculus
GGTCGATCTTCTATGATCCCCGACTAC

SEQ ID NO: 154 - protein, 9 aa, Mus musculus
GRSSMIPDY

SEQ ID NO: 155 - DNA, 414 bp, Mus musculus
atgaacttcgggttcagcttgattttccttgtccttgttttaaaaggtgtccagtgtgaagtgaagctggtggagtctgggggaggcttagtgaagcctggagggtccctgaacctctcctgtgcagcctctggattcactttcagtaactatgccatgtcttggtctcgccagactccagagaagaggctggagtgggtcgcatccattagtagtggtggtaacacctactatccagacagtgtgaggggccgattcaccatctccagagataatgccaggaacatcctgtacctgcaaatgagcagtctgaggtctgaggacacggccatgtattactgtgcaagagagcccggtacctacaactggtacttcgatgtctggggcgcagggaccacggtcaccgtctcctca

SEQ ID NO: 156 - protein, 138 aa, Mus musculus
MNFGFSLIFLVLVLKGVQCEVKLVESGGGLVKPGGSLNLSCAASGFTFSNYAMSWSRQTPEKRLEWVASISSGGNTYYPDSVRGRFTISRDNARNILYLQMSSLRSEDTAMYYCAREPGTYNWYFDVWGAGTTVTVSS

SEQ ID NO: 157 - DNA, 30 bp, Mus musculus; identical to SEQ ID NO: 69

SEQ ID NO: 158 - protein, 10 aa, Mus musculus
GFTFSNYAMS

SEQ ID NO: 159 - DNA, 48 bp, Mus musculus
tccattagtagtggtggtaacacctactatccagacagtgtgaggggc

SEQ ID NO: 160 - protein, 16 aa, Mus musculus
SISSGGNTYYPDSVRG

SEQ ID NO: 161 - DNA, 33 bp, Mus musculus
gagcccggtacctacaactggtacttcgatgtc

SEQ ID NO: 162 - protein, 11 aa, Mus musculus
EPGTYNWYFDV

SEQ ID NO: 163 - DNA, 420 bp, Mus musculus
atggctgtcttggggctgctcttctgcctggtgacattcccaagctgtgtcctatcccaggtgcagctgaagcagtcaggacctggcctagtgcagccctcacagagcctgtccatcacctgcacagtctctggtttctcattaactacctatggtgtacactggtttcgccagtctccaggaaagggtctggagtggctgggagtgatatggagtggtggaagcacagactttaatgcagctttcatatccagactgaccatcaacaaggacaattccaagagccaagttttctttacaatgaacagtctgcaagctaatgacaccgccatatattactgtgccagaattagtacggtactagccaattactatcctttggactactggggtcaaggaacctcagtcaccgtctcctca

SEQ ID NO: 164 - protein, 140 aa, Mus musculus
MAVLGLLFCLVTFPSCVLSQVQLKQSGPGLVQPSQSLSITCTVSGFSLTTYGVHWFRQSPGKGLEWLGVIWSGGSTDFNAAFISRLTINKDNSKSQVFFTMNSLQANDTAIYYCARISTVLANYYPLDYWGQGTSVTVSS

SEQ ID NO: 165 - DNA, 30 bp, Mus musculus; identical to SEQ ID NO: 117

SEQ ID NO: 166 - protein, 10 aa, Mus musculus
GFSLTTYGVH

SEQ ID NO: 167 - DNA, 48 bp, Mus musculus
gtgatatggagtggtggaagcacagactttaatgcagctttcatatcc

SEQ ID NO: 168 - protein, 16 aa, Mus musculus
VIWSGGSTDFNAAFIS

SEQ ID NO: 169 - DNA, 39 bp, Mus musculus
attagtacggtactagccaattactatcctttggactac

SEQ ID NO: 170 - protein, 13 aa, Mus musculus
ISTVLANYYPLDY

SEQ ID NO: 171 - DNA, 33 bp, Mus musculus
aagaccagtcagagtgtgagtaatgatgtagct

SEQ ID NO: 172 - protein, 11 aa, Mus musculus
KTSQSVSNDVA

SEQ ID NO: 173 - DNA, 21 bp, Mus musculus
tatgcatccaatcgctacact

SEQ ID NO: 174 - protein, 7 aa, Mus musculus
YASNRYT

SEQ ID NO: 175 - DNA, 27 bp, Mus musculus
caccaggattataggtctccgctcacg

SEQ ID NO: 176 - protein, 9 aa, Mus musculus
HQDYRSPLT

SEQ ID NO: 177 - DNA, 363 bp, Mus musculus
gaggtccagctgcaacagtctggacctgagctggtgaagcctggagcttcaatgaagatatcctgcaaggcttctggttactcattcactggctacaccatgacctgggtgaagcagagccatggaaagaaccttgagtggattggacttattaatccttacattggtgttactagctacaaccagaagttcaagggcaaggccacattgactgtagacaagtcatccagcacagcctacatggagctcctcagtctgacatctgaggactctgcagtctattactgtgcaagactctatgatcgaggtggacgctatgctatggactactggggtcaaggaacctcagtcaccgtctcctca

SEQ ID NO: 178 - protein, 121 aa, Mus musculus
EVQLQQSGPELVKPGASMKISCKASGYSFTGYTMTWVKQSHGKNLEWIGLINPYIGVTSYNQKFKGKATLTVDKSSSTAYMELLSLTSEDSAVYYCARLYDRGGRYAMDYWGQGTSVTVSS

SEQ ID NO: 179 - DNA, 30 bp, Mus musculus
ggttactcattcactggctacaccatgacc

SEQ ID NO: 180 - protein, 10 aa, Mus musculus
GYSFTGYTMT

SEQ ID NO: 181 - DNA, 51 bp, Mus musculus
cttattaatccttacattggtgttactagctacaaccagaagttcaagggc

SEQ ID NO: 182 - protein, 17 aa, Mus musculus
LINPYIGVTSYNQKFKG

SEQ ID NO: 183 - DNA, 36 bp, Mus musculus
ctctatgatcgaggtggacgctatgctatggactac

SEQ ID NO: 184 - protein, 12 aa, Mus musculus
LYDRGGRYAMDY

SEQ ID NO: 185 - DNA, 309 bp, Mus musculus
caaattgttctcacccagtctccagcaatcatgtctgcatctccaggggagaaggtccccatgacctgcagtgccagctcaagtgtaagtttcatgcactggtaccagcagaagtcaggcacctcccccaaaagatggatttatgacacatccaaactggcttctggagtccctgctcgcttcagtggcagtgggtctgggacctcttactctctcacaatcagcaacatggaggctgaagatgctgccacttattactgccagcagtggaatagtgacccgtggacgttcggtggaggctccaagctt

SEQ ID NO: 186 - protein, 103 aa, Mus musculus
QIVLTQSPAIMSASPGEKVPMTCSASSSVSFMHWYQQKSGTSPKRWIYDTSKLASGVPARFSGSGSGTSYSLTISNMEAEDAATYYCQQWNSDPWTFGGGSKL

SEQ ID NO: 187 - DNA, 30 bp, Mus musculus
agtgccagctcaagtgtaagtttcatgcac

SEQ ID NO: 188 - protein, 10 aa, Mus musculus
SASSSVSFMH

SEQ ID NO: 189 - DNA, 21 bp, Mus musculus
gacacatccaaactggcttct

SEQ ID NO: 190 - protein, 7 aa, Mus musculus
DTSKLAS

SEQ ID NO: 191 - DNA, 27 bp, Mus musculus
cagcagtggaatagtgacccgtggacg

SEQ ID NO: 192 - protein, 9 aa, Mus musculus
QQWNSDPWT

SEQ ID NO: 193 - DNA, 420 bp, Mus musculus
atggaatggctgtggaacttgctatttctcatggcagcagctcaaagtatccaagcacagatccagttggtgcagtctggacctgagctgaagaagcctggagagacagtcaggatctcctgcaaggcttctgggtattccttcacaactgctggaatgcagtgggtgcaagagatgccaggaaagggtttgaagtggattggctggatagacacccactctgcagtgccaaaatatgcagaagacttcaagggacggtttgccttctctttggaaacctctgccagcactgcatatttacagataagcaacctcaaaaatgaggacacggctgcgtatttctgtgcgagatctcaggccgactatggtaactacggatttgattactggggccaagggactctggtcactgtgtctgca

SEQ ID NO: 194 - protein, 140 aa, Mus musculus
MEWLWNLLFLMAAAQSIQAQIQLVQSGPELKKPGETVRISCKASGYSFTTAGMQWVQEMPGKGLKWIGWIDTHSAVPKYAEDFKGRFAFSLETSASTAYLQISNLKNEDTAAYFCARSQADYGNYGFDYWGQGTLVTVSA

SEQ ID NO: 195 - DNA, 30 bp, Mus musculus
gggtattccttcacaactgctggaatgcag

SEQ ID NO: 196 - protein, 10 aa, Mus musculus
GYSFTTAGMQ

SEQ ID NO: 197 - DNA, 51 bp, Mus musculus
tggatagacacccactctgcagtgccaaaatatgcagaagacttcaaggga

SEQ ID NO: 198 - protein, 17 aa, Mus musculus
WIDTHSAVPKYAEDFKG

SEQ ID NO: 199 - DNA, 36 bp, Mus musculus
tctcaggccgactatggtaactacggatttgattac

SEQ ID NO: 200 - protein, 12 aa, Mus musculus
SQADYGNYGFDY

SEQ ID NO: 201 - protein, 416 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPT

SEQ ID NO: 202 - protein, 414 aa, Mus musculus
MGTGTLSSLLLLLLLVTIGDADMKGHFDPAKCRYALGMQDRTIPDSDISVSSSWSDSTAARHSRLESSDGDGAWCPAGPVFPKEEEYLQVDLRRLHLVALVGTQGRHAGGLGKEFSRSYRLRYSRDGRRWMDWKDRWGQEVISGNEDPGGVVLKDLGPPMVARLVRFYPRADRVMSVCLRVELYGCLWRDGLLSYTAPVGQTMQLSEVMVHLNDSTYDGYTAGGLQYGGLGQLADGVVGLDDFRQSQELRVWPGYDYVGWSNQSFPTGYVEMEFEFDRLRTFQTMQVHCNNMHTLGARLPGGVECRFKRGPAMAWEGEPVRHALGGSLGDPRARAISVPLGGHVGRFLQCRFLFAGPWLLFSEISFISDVVNDSSDTFPPAPWWPPGPPPTNFSSLELEPRGQQPVAKAEGSPT

SEQ ID NO: 203 - protein, 119 aa, Homo sapiens
EVQLVESGGGLVQPGGSLRFSCAASGFTFSSYGMSWVRQAPGKGLELVAIINSHGFSTYYPDSVKGRFTISRDNAKNTLYLQMSSLRAEDTAIYYCARGGYGNYGMDYWGQGTSVTVSS

SEQ ID NO: 204 - protein, 119 aa, Homo sapiens
EVQLVESGGGLVQPGGSLRFSCAASGFTFSSYGMSWVRQAPGKGLELVAIINSHGFSTYYPDSVKGRFTISRDNAKNTLYLQMNSLRAEDTAIYYCARGGYGNYGMDYWGQGTTVTVSS

SEQ ID NO: 205 - protein, 119 aa, Homo sapiens
EVQLVESGGGLVQPGGSLRLSCAASGFTFSSYGMSWVRQAPGKGLELVAIINSHGFSTYYPDSVKGRFTISRDNAKNTLYLQMNSLRAEDTAIYYCARGGYGNYGMDYWGQGTTVTVSS

SEQ ID NO: 206 - protein, 119 aa, Homo sapiens
EVQLVESGGGLVQPGGSLRLSCAASGFTFSSYGMSWVRQAPGKGLELVAIINSHGFSTYYPDSVKGRFTISRDNAKNTLYLQMNSLRAEDTAVYYCARGGYGNYGMDYWGQGTTVTVSS

SEQ ID NO: 207 - protein, 111 aa, Homo sapiens
DIVLTQSPASLALSPGERATLSCRASESVDSYGNSFMHWYQQKVGKPPKLLIFRASNIESGIPARFSGSGSRTDFTLTISSLQAEDFATYYCQQSNEAPYTFGQGTKLEIK

SEQ ID NO: 208 - protein, 111 aa, Homo sapiens
DIVLTQSPATLSLSPGERATLSCRASESVDSYGNSFMHWYQQKVGKAPKLLIFRASNIESGIPARFSGSGSRTDFTLTISSLQPEDFATYYCQQSNEAPYTFGQGTKLEIK

SEQ ID NO: 209 - protein, 111 aa, Homo sapiens
DIVLTQSPATLSLSPGERATLSCRASESVDSYGNSFMHWYQQKPGKAPKLLIFRASNIESGIPARFSGSGSRTDFTLTISSLQPEDFATYYCQQSNEAPYTFGQGTKLEIK

SEQ ID NO: 210 - protein, 111 aa, Homo sapiens
DIVLTQSPATLSLSPGERATLSCRASESVDSYGNSFMHWYQQKPGKAPKLLIFRASNIESGIPARFSGSGSRTDFTLTISSLQPEDFAVYYCQQSNEAPYTFGQGTKLEIK

SEQ ID NO: 211 - DNA, 384 bp, Mus musculus
atggagacacattctcaggtctttgtatacatgttgctgtggttgtctggtgttgaaggagacattgtgatgacccagtctcacaaattcatgtccacatcagtaggagacagggtcagcatcacctgcaaggccagtcaggatgtgggtactgctgtagcctggtatcaacagaaaccaggacaatctcctaaactactgatttactgggcatccacccggcacactggagtccctgatcgcttcacaggcagtggatctgggacagatttcactctcaccattagcaatgtgcagtctgaagacttggcagattatttctgtcagcaatacagcacctatcctctcacgttcggtgctgggaccaagctggagctgaaacgg

SEQ ID NO: 212 - protein, 128 aa, Mus musculus
METHSQVFVYMLLWLSGVEGDIVMTQSHKFMSTSVGDRVSITCKASQDVGTAVAWYQQKPGQSPKLLIYWASTRHTGVPDRFTGSGSGTDFTLTISNVQSEDLADYFCQQYSTYPLTFGAGTKLELKR

SEQ ID NO: 213 - DNA, 33 bp, Mus musculus
aaggccagtcaggatgtgggtactgctgtagcc

SEQ ID NO: 214 - protein, 11 aa, Mus musculus
KASQDVGTAVA

SEQ ID NO: 215 - DNA, 21 bp, Mus musculus
tgggcatccacccggcacact

SEQ ID NO: 216 - protein, 7 aa, Mus musculus
WASTRHT

SEQ ID NO: 217 - DNA, 27 bp, Mus musculus
cagcaatacagcacctatcctctcacg

SEQ ID NO: 218 - protein, 9 aa, Mus musculus
QQYSTYPLT

SEQ ID NO: 219 - DNA, 384 bp, Mus musculus
atggagtcacagactcaggtctttgtatacatgttgctgtggttgtctggtgttgatggagacattgtgatgacccagtctcaaaaattcatgtccacatcagtaggagacagggtcagcgtcacctgcaaggccagtcagaatgtgggtactaatgtagcctggtatcaacagaaaccagggcaatctcttaaagcactgatttactcggcatcctaccggttcagtggagtccctgatcgcttcacaggcagtggatatgggacagatttcactctcaccatcagcaatgtgcagtctgaagacttggcagagtatttctgtcagcaatgtaacagctatcctctcacgttcggtgctgggaccaagctggagctgaaacgg

SEQ ID NO: 220 - protein, 128 aa, Mus musculus
MESQTQVFVYMLLWLSGVDGDIVMTQSQKFMSTSVGDRVSVTCKASQNVGTNVAWYQQKPGQSLKALIYSASYRFSGVPDRFTGSGYGTDFTLTISNVQSEDLAEYFCQQCNSYPLTFGAGTKLELKR

SEQ ID NO: 221 - DNA, 33 bp, Mus musculus
aaggccagtcagaatgtgggtactaatgtagcc

SEQ ID NO: 222 - protein, 11 aa, Mus musculus
KASQNVGTNVA

SEQ ID NO: 223 - DNA, 21 bp, Mus musculus
tcggcatcctaccggttcagt

SEQ ID NO: 224 - protein, 7 aa, Mus musculus
SASYRFS

SEQ ID NO: 225 - DNA, 27 bp, Mus musculus
cagcaatgtaacagctatcctctcacg

SEQ ID NO: 226 - protein, 9 aa, Mus musculus
QQCNSYPLT

SEQ ID NO: 227 - protein, 119 aa, Homo sapiens
QVQLHQSGSELAKPGASVKMSCKASGYIFTTYWIHWLKQRPGQGLEWIGFINPDTDYTEYNQKFKDKATLTADKSSNTAYVQLSSLTSEDSAVYYCARGNYGYDGLVYWGLGTLVTVSA

SEQ ID NO: 228 - protein, 119 aa, Homo sapiens
QVQLVQSGSELKKPGASVKMSCKASGYIFTTYWIHWLKQAPGQGLEWIGFINPDTDYTEYNQKFKDRATLTADKSTNTAYVELSSLTSEDSAVYYCARGNYGYDGLVYWGQGTLVTVSS

SEQ ID NO: 229 - protein, 119 aa, Homo sapiens
QVQLVQSGSELKKPGASVKMSCKASGYIFTTYWIHWLKQAPGQGLEWIGFINPDTDYTEYNQKFKDRATLTADKSTNTAYVELSSLRSEDTAVYYCARGNYGYDGLVYWGQGTLVTVSS

SEQ ID NO: 230 - protein, 119 aa, Homo sapiens
QVQLVQSGSELKKPGASVKMSCKASGYIFTTYWIHWLKQAPGQGLEWIGFINPDTDYTEYNQKFKDRATLTADKSTSTAYVELSSLRSEDTAVYYCARGNYGYDGLVYWGQGTLVTVSS

SEQ ID NO: 231 - protein, 119 aa, Homo sapiens
QVQLVQSGSELKKPGASVKMSCKASGYIFTTYWIHWLRQAPGQGLEWIGFINPDTDYTEYNQKFKDRATLTADKSTSTAYVELSSLRSEDTAVYYCARGNYGYDGLVYWGQGTLVTVSS

SEQ ID NO: 232 - protein, 119 aa, Homo sapiens
QVQLVQSGSELKKPGASVKVSCKASGYIFTTYWIHWLRQAPGQGLEWIGFINPDTDYTEYNQKFKDRATITADKSTSTAYMELSSLRSEDTAVYYCARGNYGYDGLVYWGQGTLVTVSS

SEQ ID NO: 233 - protein, 106 aa, Homo sapiens
SIVMTQTPKFLLVSAGDRVTITCKASQSVSTDVAWYQQKPGQSPKLLIYYASNRYTGVPDRFTGSGYGTDFTFTISTVQAEDLAVYFCQQDYNSYTFGGGTKLEIK

SEQ ID NO: 234 - protein, 106 aa, Homo sapiens
SIVMTQSPSFLLASVGDRVTITCKASQSVSTDVAWYQQKPGKAPKLLIYYASNRYTGVPDRFTGSGYGTDFTFTISSLQAEDVAVYFCQQDYNSYTFGQGTKLEIK

SEQ ID NO: 235 - protein, 106 aa, Homo sapiens
SIVMTQSPSFLSASVGDRVTITCKASQSVSTDVAWYQQKPGKAPKLLIYYASNRYTGVPDRFTGSGYGTDFTLTISSLQAEDVAVYFCQQDYNSYTFGQGTKLEIK

SEQ ID NO: 236 - protein, 106 aa, Homo sapiens
SIVMTQSPSFLSASVGDRVTITCKASQSVSTDVAWYQQKPGKAPKLLIYYASNRYTGVPDRFSGSGYGTDFTLTISSLQAEDVAVYFCQQDYNSYTFGQGTKLEIK

SEQ ID NO: 237 - protein, 106 aa, Homo sapiens
SIQMTQSPSFLSASVGDRVTITCKASQSVSTDVAWYQQKPGKAPKLLIYYASNRYTGVPDRFSGSGYGTDFTLTISSLQAEDVAVYFCQQDYNSYTFGQGTKLEIK

(19) United States
(12) Patent Application Publication
(10) Pub. No.: US 2014/0086913 A1
(43) Pub. Date: Mar. 27, 2014

(54) ANTI-DDR1 ANTIBODIES

(71) Applicant: GILEAD SCIENCES, INC., FOSTER CITY, CA (US)

(72) Inventors: Victoria SMITH, Burlingame, CA (US); Scott Alan MCCAULEY, Brisbane, CA (US); Maria VAYSBERG, Los Altos, CA (US); Joanne I. ADAMKEWICZ, Belmont, CA (US)

(73) Assignee: GILEAD SCIENCES, INC., FOSTER CITY, CA (US)

(21) Appl. No.: 14/037,081

(22) Filed: Sep. 25, 2013

Related U.S. Application Data
(63) Continuation of application No. PCT/US2013/061390, filed on Sep. 24, 2013.
(60) Provisional application No. 61/800,450, filed on Mar. 15, 2013.
(60) Provisional application No. 61/705,044, filed on Sep. 24, 2012.

(51) Int. Cl. C07K 16/28; G01N 33/68

(52) U.S. Cl. CPC C07K 16/2863; G01N 33/68
USPC 424/133.1; 530/389.2; 530/387.3; 424/130.1; 424/158.1; 436/501; 435/7.92

(57) ABSTRACT

Provided are antibodies, including functional antibody fragments, that specifically bind to discoidin domain receptors (DDRs), and in particular to DDR1 proteins, as well as uses and method of using such antibodies, including in the detection, diagnosis and treatment of diseases and conditions associated with DDR1.

### CROSS-REFERENCE TO RELATED APPLICATIONS

This application is a continuation of International Application No. PCT/US2013/061390, filed Sep. 24, 2013, which application claims the benefit under 35 U.S.C. §119(e) of U.S. Provisional Application No. 61/800,450, filed Mar. 15, 2013 and U.S. Provisional Application No. 61/705,044, filed Sep. 24, 2012, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is GILE_{—}050_{—}02US_ST25.txt. The text file is 113 KB, was created on Sep. 25, 2013, and is being submitted electronically via EFS-Web, concurrent with the filing of the specification.

### TECHNICAL FIELD

The present disclosure relates in some aspects to antibodies, including functional antibody fragments, that bind DDR1. The present disclosure further relates to uses and methods of using such antibodies, including in the detection, diagnosis and treatment of diseases and conditions associated with DDR1.

### BACKGROUND

DDR1 (also known as CAK, CD167a, RTK6, and TrkE) belongs to the discoidin-like domain containing subfamily of receptor tyrosine kinases, which also includes DDR2. Vogel et al., Molecular Cell, Vol. 1, 13-23, December, 1997; Vogel et al., The FASEB Journal, Vol. 13, Supplement, s77-s82, 1999; Yoshimura et al., Immunologic Research, 31(3): 219-29. DDR1 encompasses multiple isoforms, resulting from alternative splicing, including DDR1a and DDR1b. Id. DDR1 proteins contain an extracellular domain (ECD) that shares some homology with DDR2. Vogel et al., Molecular Cell, Vol. 1, 13-23, December, 1997. DDR1 is involved in a number of diseases and conditions. Vogel et al., Molecular Cell, Vol. 1, 13-23, December, 1997; Vogel et al., The FASEB Journal, Vol. 13, Supplement, s77-s82, 1999; Yoshimura et al., Immunologic Research, 31(3): 219-29. There is a need for DDR1 inhibitors and antibodies that specifically bind to DDR1, such as antibodies that inhibit DDR1, and therapeutic, diagnostic, and prognostic methods using the same.

### SUMMARY

Provided herein are antibodies, including antibody fragments, generally isolated antibodies, that specifically bind to discoidin domain receptors (DDRs), generally to discoidin domain receptor family, member 1 (DDR1), and methods and uses of the same, including therapeutic, detection, diagnostic, and prognostic methods and uses. In some embodiments, the antibody inhibits DDR1, e.g., the activity of DDR1.

The antibody typically includes one or more heavy chain complementarity determining region (CDRH). In one embodiment, the CDRH is a CDRH selected from the group consisting of: a) the heavy chain variable (VH) region comprising the amino acid sequence set forth as SEQ ID NO: 4; b) the VH region comprising the amino acid sequence set forth as SEQ ID NO: 20; c) the VH region comprising the amino acid sequence set forth as SEQ ID NO: 36; d) the VH region comprising the amino acid sequence set forth as SEQ ID NO: 52; e) the VH region comprising the amino acid sequence set forth as SEQ ID NO: 68; f) the VH region comprising the amino acid sequence set forth as SEQ ID NO: 84; g) the VH region comprising the amino acid sequence set forth as SEQ ID NO: 100; h) the VH region comprising the amino acid sequence set forth as SEQ ID NO:116; i) the VH region comprising the amino acid sequence set forth as SEQ ID NO:132; j) the VH region comprising the amino acid sequence set forth as SEQ ID NO:148; k) the VH region comprising the amino acid sequence set forth as SEQ ID NO:156; 1) the VH region comprising the amino acid sequence set forth as SEQ ID NO:164; m) the VH region comprising the amino acid sequence set forth as SEQ ID NO:178; n) the VH region comprising the amino acid sequence set forth as SEQ ID NO:194; o) the VH region comprising the amino acid sequence set forth as SEQ ID NO:203, p) the VH region comprising the amino acid sequence set forth as SEQ ID NO:204; q) the VH region comprising the amino acid sequence set forth as SEQ ID NO:205; r) the VH region comprising the amino acid sequence set forth as SEQ ID NO:206; s) the VH region comprising the amino acid sequence set forth as SEQ ID NO:227; t) the VH region comprising the amino acid sequence set forth as SEQ ID NO:228; u) the VH region comprising the amino acid sequence set forth as SEQ ID NO:229; v) the VH region comprising the amino acid sequence set forth as SEQ ID NO:230; w) the VH region comprising the amino acid sequence set forth as SEQ ID NO:231; and x) the VH region comprising the amino acid sequence set forth as SEQ ID NO:232.

In some embodiments, the one or more CDRH includes a CDRH3, such as a CDRH3 having the amino acid sequence set forth as SEQ ID NO: 10, 26, 42, 58, 74, 90, 106, 122, 138, 154, 162, 170, 184, or 200 or an amino acid sequence having at least at or about 75%, 80%, 85%, 90%, 95%, 96%, 97%, 98%, or 99% identity to such a sequence. In some embodiments, the one or more CDRH1 includes a CDRH1 and/or a CDRH2, such as a CDRH1 having the amino acid sequence set forth as SEQ ID NO: 6, 22, 38, 54, 70, 86, 102, 118, 134, 150, 158, 166, or 180 and/or the CDRH2 comprises the amino acid sequence set forth as SEQ ID NO: 8, 24, 40, 56, 72, 88, 104, 120, 136, 152, 160, 168, 182, or 198 or an amino acid sequence having at least at or about 75%, 80%, 85%, 90%, 95%, 96%, 97%, 98%, or 99% identity to such a sequence.

In some aspects, the antibody includes a VH region comprising the amino acid sequence set forth in SEQ ID NO: 4, 20, 36, 52, 68, 84, 100, 116, 132, 148, 156, 164, 178, 194, 203, 204, 205, 206, 227, 228, 229, 230, 231, or 232, with or without the leader sequence, or an amino acid sequence having at least at or about 75%, 80%, 85%, 90%, 95%, 96%, 97%, 98%, or 99% identity to such a sequence.

In some embodiments, the antibody comprises one or more light chain complementarity determining region (CDRL). In some examples, the one or more CDRL is selected from the group consisting of a) the light chain variable (VL) region comprising the amino acid sequence set forth as SEQ ID NO: 12; b) the VL region comprising the amino acid sequence set forth as SEQ ID NO: 28; c) the VL region comprising the amino acid sequence set forth as SEQ ID NO: 44; d) the VL region comprising the amino acid sequence set forth as SEQ ID NO: 60; e) the VL region comprising the amino acid sequence set forth as SEQ ID NO: 76; f) the VL region comprising the amino acid sequence set forth as SEQ ID NO: 92; g) the VL region comprising the amino acid sequence set forth as SEQ ID NO:108; h) the VL region comprising the amino acid sequence set forth as SEQ ID NO:124; i) the VL region comprising the amino acid sequence set forth as SEQ ID NO:140; j) the VL region comprising the amino acid sequence set forth as SEQ ID NO:186; k) the VL region comprising the amino acid sequence set forth as SEQ ID NO:207; 1) the VL region comprising the amino acid sequence set forth as SEQ ID NO:208; m) the VL region comprising the amino acid sequence set forth as SEQ ID NO: 209; n) the VL region comprising the amino acid sequence set forth as SEQ ID NO:210; o) the VL region comprising the amino acid sequence set forth as SEQ ID NO:212; p) the VL region comprising the amino acid sequence set forth as SEQ ID NO:220; q) the VL region comprising the amino acid sequence set forth as SEQ ID NO:233; r) the VL region comprising the amino acid sequence set forth as SEQ ID NO:234; s) the VL region comprising the amino acid sequence set forth as SEQ ID NO:235; t) the VL region comprising the amino acid sequence set forth as SEQ ID NO:236; and u) the VL region comprising the amino acid sequence set forth as SEQ ID NO:237.

In some embodiments, the one or more CDRL includes a CDRL3, such as a CDRL3 having the amino acid sequence set forth as SEQ ID NO: 18, 34, 50, 66, 82, 98, 114, 130, 146, 176, 192, 218, or 226, or an amino acid sequence having at least at or about 75%, 80%, 85%, 90%, 95%, 96%, 97%, 98%, or 99% identity to such a sequence. In some embodiments, the one or more CDRL includes a CDRL1 and/or a CDRL2, such as a CDRL1 having the amino acid sequence set forth as SEQ ID NO: 14, 30, 46, 62, 78, 94, 110, 126, 142, 172, 188, 214, or 222, and/or the CDRL2 having the amino acid sequence set forth as SEQ ID NO: 16, 32, 48, 64, 80, 96, 112, 128, 144, 174, 190, 216, or 224, or an amino acid sequence having at least at or about 75%, 80%, 85%, 90%, 95%, 96%, 97%, 98%, or 99% identity to such a sequence.

In some embodiments, the antibody includes a VL region having the amino acid sequence set forth in SEQ ID NO: 12, 28, 44, 60, 76, 92, 108, 124, 140, 186, 207, 208, 209, 210, 212, 220, 233, 234, 235, 236, or 237, with or without the leader sequence, or an amino acid sequence having at least at or about 75%, 80%, 85%, 90%, 95%, 96%, 97%, 98%, or 99% identity to such a sequence.

In some embodiments, the antibody has or has sequences with at least at or about 75%, 80%, 85%, 90%, 95%, 96%, 97%, 98%, or 99% identity to. a) a VH region comprising the amino acid sequence set forth as SEQ ID NO: 4, with or without the leader sequence and a VL region having the amino acid sequence set forth as SEQ ID NO: 12, with or without the leader sequence; b) a VH region comprising the amino acid sequence set forth as SEQ ID NO: 20, with or without the leader sequence and a VL region having the amino acid sequence set forth as SEQ ID NO: 28, with or without the leader sequence; c) a VH region comprising the amino acid sequence set forth as SEQ ID NO: 36, with or without the leader sequence and a VL region having the amino acid sequence set forth as SEQ ID NO: 44, with or without the leader sequence; d) a VH region comprising the amino acid sequence set forth as SEQ ID NO: 52, with or without the leader sequence and a VL region having the amino acid sequence set forth as SEQ ID NO: 60, with or without the leader sequence; e) a VH region comprising the amino acid sequence set forth as SEQ ID NO: 68, with or without the leader sequence and a VL region having the amino acid sequence set forth as SEQ ID NO: 76, with or without the leader sequence; f) a VH region comprising the amino acid sequence set forth as SEQ ID NO: 84, with or without the leader sequence and a VL region having the amino acid sequence set forth as SEQ ID NO: 92, with or without the leader sequence; g) a VH region comprising the amino acid sequence set forth as SEQ ID NO: 100, with or without the leader sequence and a VL region having the amino acid sequence set forth as SEQ ID NO: 108, with or without the leader sequence; h) a VH region comprising the amino acid sequence set forth as SEQ ID NO: 116, with or without the leader sequence and a VL region having the amino acid sequence set forth as SEQ ID NO: 124, with or without the leader sequence; i) a VH region comprising the amino acid sequence set forth as SEQ ID NO: 132, with or without the leader sequence and a VL region having the amino acid sequence set forth as SEQ ID NO: 140, with or without the leader sequence; j) a VH region comprising the amino acid sequence set forth as SEQ ID NO: 178, with or without the leader sequence and a VL region having the amino acid sequence set forth as SEQ ID NO: 186, with or without the leader sequence; k) a VH region comprising the amino acid sequence set forth as SEQ ID NO: 148, with or without the leader sequence and a VL region having the amino acid sequence set forth as SEQ ID NO: 212, with or without the leader sequence; l) a VH region comprising the amino acid sequence set forth as SEQ ID NO: 156, with or without the leader sequence and a VL region having the amino acid sequence set forth as SEQ ID NO: 220, with or without the leader sequence; m) a VH region comprising the amino acid sequence set forth as in any one of SEQ ID NO: 203, 204, 205, and 206, with or without a leader sequence and a VL region having the amino acid sequence set forth as in any one of SEQ ID NO: 207, 208, 209, and 210 with or without a leader sequence; or n) a VH region comprising the amino acid sequence set forth as in any one of SEQ ID NO: 227, 228, 229, 230, 231, and 232, with or without a leader sequence and a VL region having the amino acid sequence set forth as in any one of SEQ ID NO: 233, 234, 235, 236, and 237 with or without a leader sequence.

In certain embodiments, the anti-DDR1 antibody comprises the relevant sequences of AB2039 and/or AB2041, a humanized form thereof, or an amino acid sequence having at least at or about 75%, 80%, 85%, 90%, 95%, 96%, 97%, 98%, or 99% identity to such an antibody.

In some embodiments, the antibody competes for binding to a DDR1 protein with any of the antibodies described above. In some embodiments, the antibody is chimeric, human or humanized. In some embodiments, it is an antibody fragment, such as a Fab fragment, a Fab′ dimer, an scFv, or Fv fragment.

Also provided are methods for treating a disease or condition associated with DDR1 in a subject. In some embodiments, the methods are carried out by administering to the subject an antibody that specifically binds to DDR1, e.g., thereby treating the disease or condition in the subject. In some embodiments, the methods are carried out using any of the above-described antibodies.

Also provided are detection methods carried out by detecting DDR1 levels and/or activity in a sample from a subject using the DDR1 antibodies, such as by contacting the sample with the antibody and assessing or measuring the presence or extent of binding thereof to a protein in the sample. The methods in some embodiments further include comparing the levels, activity, and/or binding to that observed or known to be present in a control sample. In some cases, the methods indicate the presence, or severity of the disease or condition or sign or symptom thereof in the subject.

Among the diseases and conditions for use with the provided methods are cancer, including but not limited to breast cancer, lung cancer, ovarian cancer, brain cancer, esophageal cancer, bile duct cancer such as cholangiocarcinoma, metastasis, angiogenesis, tumor invasion and/or progression, diseases associated with cell proliferation, cell invasion, and/or deregulation of extracellular matrix production, and/or fibrosis such as pulmonary or lung fibrosis, and inflammatory and autoimmune diseases, such as but not limited to glomerulonephritis, rheumatoid arthritis.

### BRIEF DESCRIPTION OF THE DRAWINGS

FIG. 1 shows the alignment of the protein sequences of human (Hu) (SEQ ID NO:201) and mouse (Mu) (SEQ ID NO:202) DDR1 extracellular domains (ECD), including the collagen-binding discoidin (DS) and DS-like domains Amino acid residues in shaded blocks indicate non-conserved residues between the two species.

FIG. 2 shows a three-dimensional model of human DDR1-ECD protein, and residues involved in anti-DDR1 antibody binding.

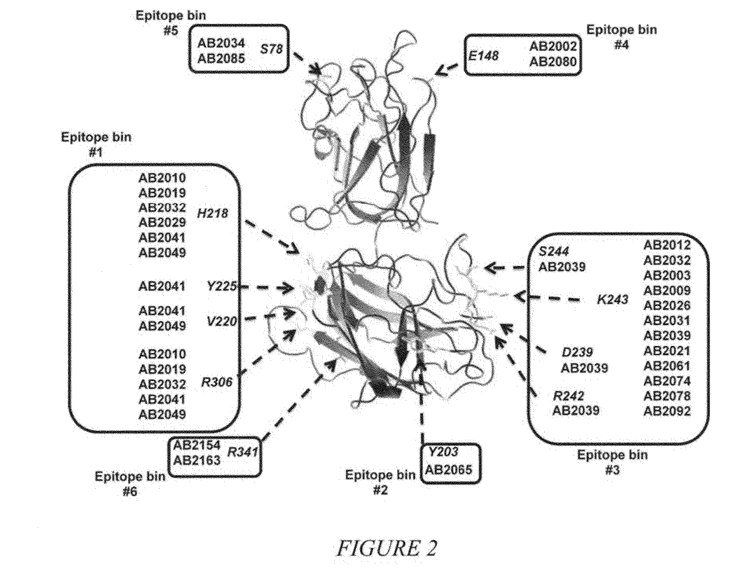

### DETAILED DESCRIPTION OF THE INVENTION

### Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

When a trade name is used herein, reference to the trade name also refers to the product formulation, the generic drug, and the active pharmaceutical ingredient(s) of the trade name product, unless otherwise indicated by context.

The term “antibody” is used in the broadest sense unless clearly indicated otherwise, and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to Fv, scFv, Fab, Fab′ F(ab′)₂ and Fab₂, so long as they exhibit the desired biological activity. The term “human antibody” refers to antibodies containing sequences of human origin, except for possible non-human CDR regions, and does not imply that the full structure of an immunoglobulin molecule be present, only that the antibody has minimal immunogenic effect in a human (i.e., does not induce the production of antibodies to itself).

An “antibody fragment” comprises a portion of a full-length antibody, for example, the antigen binding or variable region of a full-length antibody. Such antibody fragments may also be referred to herein as “functional fragments: or “antigen-binding fragments”. Examples of antibody fragments include Fab, Fab′, F(ab′)₂, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is a minimum antibody fragment containing a complete antigen-recognition and -binding site. This region consists of a dimer of one heavy- and one light-chain variable domain in tight, non-covalent association. It is in this configuration that the three complementarity-determining regions (CDRs) of each variable domain interact to define an antigen-binding site on the surface of the VH-VL dimer. Collectively, the six CDRs confer antigen-binding specificity to the antibody. However, even a single variable domain (or an isolated VH or VL region comprising only three of the six CDRs specific for an antigen) has the ability to recognize and bind antigen, although generally at a lower affinity than does the entire Fᵥ fragment.

The “Fab” fragment also contains, in addition to heavy and light chain variable regions, the constant domain of the light chain and the first constant domain (CH₁) of the heavy chain. Fab fragments were originally observed following papain digestion of an antibody. Fab′ fragments differ from Fab fragments in that F(ab′) fragments contain several additional residues at the carboxy terminus of the heavy chain CH₁ domain, including one or more cysteines from the antibody hinge region. F(ab′)₂ fragments contain two Fab fragments joined, near the hinge region, by disulfide bonds, and were originally observed following pepsin digestion of an antibody. Fab′-SH is the designation herein for Fab′ fragments in which the cysteine residue(s) of the constant domains bear a free thiol group. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to five major classes: IgA, IgD, IgE, IgG, and IgM, and several of these may be further divided into subclasses (isotypes), e.g., IgG1, IgG2, IgG3, IgG4, IgA1, and IgA2.

“Single-chain Fv” or “sFv” or “scFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun, in The Pharmacology of Monoclonal Antibodies, vol. 113 (Rosenberg and Moore eds.) Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain, thereby creating two antigen-binding sites. Diabodies are additionally described, for example, in EP 404,097; WO 93/11161 and Hollinger et al. (1993) Proc. Natl. Acad. Sci. USA 90:6444-6448.

An “isolated” antibody is one that has been identified and separated and/or recovered from a component of its natural environment. Components of its natural environment may include enzymes, hormones, and other proteinaceous or nonproteinaceous solutes. In some embodiments, an isolated antibody is purified (1) to greater than 95% by weight of antibody as determined by the Lowry method, for example, more than 99% by weight, (2) to a degree sufficient to obtain at least 15 residues of N-terminal or internal amino acid sequence, e.g., by use of a spinning cup sequenator, or (3) to homogeneity by gel electrophoresis (e.g., SDS-PAGE) under reducing or nonreducing conditions, with detection by Coomassie blue or silver stain. The term “isolated antibody” includes an antibody in situ within recombinant cells, since at least one component of the antibody's natural environment will not be present. In certain embodiments, isolated antibody is prepared by at least one purification step.

As used herein, “immunoreactive” refers to antibodies or fragments thereof that are specific to a sequence of amino acid residues (“binding site” or “epitope”), yet if are cross-reactive to other peptides/proteins, are not toxic at the levels at which they are formulated for administration to human use. “Epitope” refers to that portion of an antigen capable of forming a binding interaction with an antibody or antigen binding fragment thereof. An epitope can be a linear peptide sequence (i.e., “continuous”) or can be composed of noncontiguous amino acid sequences (i.e., “conformational” or “discontinuous”). The term “preferentially binds” means that the binding agent binds to the binding site with greater affinity than it binds unrelated amino acid sequences.

The terms “complementarity determining region,” and “CDR,” are known in the art to refer to non-contiguous sequences of amino acids within antibody variable regions, which confer antigen specificity and binding affinity. In general, there are three (3) CDRs in a heavy chain variable region (CDRH1, CDRH2, CDRH3) and three (3) CDRs in a light chain variable region (CDRL1, CDRL2, CDRL3).

The precise amino acid sequence boundaries of a given CDR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745.” (Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Plückthun A, “Yet another numbering scheme for immunoglobulin variable domains: an automatic modeling and analysis tool,” J Mol Biol, 2001 Jun. 8; 309(3):657-70, (“AHo” numbering scheme).

The boundaries of a given CDR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. Table V, infra, lists the positions of CDRL1, CDRL2, CDRL3 and CDRH1, CDRH2, CDRH3 as identified by the Kabat, Chothia, and Contact schemes, respectively. For CDR-H1, residue numbering is given listed using both the Kabat and Chothia numbering schemes.

Thus, unless otherwise specified, the terms “CDR” and “complementary determining region” of a given antibody or region thereof, such as a variable region, as well as individual CDRs (e.g., CDRH1, CDRH2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

As used herein, “treat” or “treatment” means stasis or a postponement of development of one or more symptoms associated with a disease or disorder described herein, or ameliorating existing uncontrolled or unwanted symptoms, preventing additional symptoms, or ameliorating or preventing the underlying metabolic causes of symptoms. Thus, the terms denote that a beneficial result has been conferred on a mammalian subject with a disease or symptom, or with the potential to develop such disease or symptom. A response is achieved when the subject experiences partial or total alleviation, or reduction of one or more signs or symptoms of disease, condition, or illness, such as, but not limited to, prolongation of survival, or reduction of tumor progression, tumor growth, metastasis, invasion, or angiogenesis, or other symptom.

As used herein, unless otherwise specified, the term “therapeutically effective amount” or “effective amount” refers to an amount of an agent or compound or composition that when administered (either alone or in combination with another therapeutic agent, as may be specified) to a subject is effective to prevent or ameliorate the disease condition or the progression of the disease, or result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

As used herein, the term “subject” means a mammalian subject. Exemplary subjects include, but are not limited to humans, monkeys, dogs, cats, mice, rats, cows, horses, goats and sheep. In certain embodiments, the subject is a human. In some embodiments, the subject has cancer, an inflammatory disease or condition, or an autoimmune disease or condition, and can be treated with the agent or the antibody of the present application. Other embodiments provide that a human in need of treatment with the antibodies of the present application, wherein the human has or is suspected to have cancer, an inflammatory disease or condition, or an autoimmune disease or condition, or a fibrotic disease or condition.

Antibodies

Provided are antibodies (including antibody fragments) that bind to discoidin domain receptors (DDRs), particularly the antibodies that specifically bind to discoidin domain receptor family, member 1 (DDR1) proteins. Suitable anti-DDR1 antibodies may be inhibitory or non-inhibitory, as both categories of antibodies have utility. In some embodiments, the antibodies specifically bind within one or more domain of DDR1, such as all or part of the extracellular domain (ECD), such as a domain comprising residues 41 through 416 of a DDR1 protein sequence, such as of SEQ ID NO: 1 or SEQ ID NO: 201. The DDR1 proteins include human DDR1 proteins, including a-isoform, b-isoform, and c-isoform of DDR1. The extracellular domain (ECD) of a-isoform and b-isoform are identical. In some embodiment, the anti-DDR1 antibodies of the present application binds to a-, b-, and c-isoforms of DDR1. In one example, the antibody binds to a DDR1 protein or polypeptide comprising the amino acid sequence referenced at GenBank gi Number 47125290 (SEQ ID NO: 1) or gi Number 83977450 (also known as NP_{—}054699), or a natural variant or homolog thereof, or a domain thereof, such as the extracellular domain. For example, in one embodiment, the DDR1 protein or polypeptide includes an amino acid sequence set forth in SEQ ID NO: 1, 2, 201, or 202. DDR1 proteins are described, for example, in Vogel et al., Molecular Cell, Vol. 1, 13-23, December, 1997.

In some embodiments, the antibodies inhibit a DDR1 protein, e.g., inhibit the activity of DDR1. It is expected but not required that inhibitory anti-DDR1 antibodies would have utility as therapeutic reagents. Inhibition by an anti-DDR1 antibody can be measured by any assay that is commonly employed by those of ordinary skill in the art and specific assays are discussed below. A variety of effective concentrations of inhibitory anti-DDR1 antibodies are reported here. In one embodiment, the anti-DDR1 antibodies described here have an EC₅₀ of less than 10 nM, less than 5 nM, less than 4 nM, less than 3 nM, less than 2 nM, or less than 1 nM.

The antibodies of the present application exhibit competitive or non-competitive inhibition to DDR1 protein. Anti-DDR1 antibodies that interact and/or bind with residues of DDR1 located distal to the collagen-binding loops are unlikely to be competitive to collagen binding, and anti-DDR1 antibodies that interact and/or bind with residues of DDR1 located in close proximity to the collagen-binding loops are likely to be competitive to collagen binding. In certain embodiment, the anti-DDR1 antibodies bind and/or inhibit DDR1 in the presence of collagen. In some embodiment, the anti-DDR1 antibodies bind and/or inhibit a complex of DDR1-collagen. In one embodiment, the anti-DDR1 antibodies exhibit non-competitive inhibition (i.e. allosteric binding or interaction) to DDR1 protein. In other embodiment, the anti-DDR1 antibodies exhibit competitive inhibition (i.e. non-allosteric binding or interaction) to DDR1 protein.

In some embodiments, the anti-DDR1 antibodies described here are non-inhibitory antibodies that bind specifically to the DDR1 protein. Such non-inhibitory antibodies have utility, for example as reagents for assay purposes.

In some aspects, the antibodies specifically bind to DDR1 and do not bind to another given discoidin domain receptor, such as DDR2, or do not exhibit detectable binding to such a receptor. For example, the antibodies described here may have an affinity which is one, two, three, four, five, ten, twenty, thirty, forty, fifty or more times greater for DDR1 than for DDR2.

In some aspects, the antibodies bind to the DDR1 with a K_{d} of no more than at or about 0.1, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.46, 0.48, 0.49, 0.5, 0.51, 0.52, 0.53, 0.54, 0.55, 0.56, 0.57, 0.58, 0.59, 0.6, 0.7, 0.8, 0.9, or 1 nM². In some cases, the K_{d} is measured via immunoassay, such as via ELISA. In some examples, the K_{d} is measured with respect to a DDR1 fusion protein, such as Fc-ECD-DR1 (R&D Systems).

In some embodiments, the antibody contains one, two, or three heavy chain CDR (CDRH) of a heavy chain variable (VH) region set forth herein, as determined by any known method, such as those described herein. In some embodiments, the antibody contains one or more CDRH of a VH region having an amino acid sequence set forth in SEQ ID NO: 4, 20, 36, 52, 68, 84, 100, 116, 132, 148, 156, 164, 178, 194, 203, 204, 205, 206, 227, 228, 229, 230, 231, or 232, or of a VH region encoded by the nucleotide sequence set forth in SEQ ID NO: 3, 19, 35, 51, 67, 83, 99, 115, 131, 147, 155, 227, 163, 177, or 193 such as the CDRH1, CDRH2, and/or CDRH3 of such a sequence, as determined by any known numbering scheme for identifying CDRs, such as any described herein.

For example, in some embodiments, the antibody contains a CDRH3 having the amino acid sequence set forth in SEQ ID NO: 10, 26, 42, 58, 74, 90, 106, 122, 138, 154, 162, 170, 184, or 200 or encoded by the nucleotide sequence set forth in SEQ ID NO: 9, 25, 41, 57, 73, 89, 105, 121, 137, 153, 161, 169, 183, or 199, a CDRH1 having the amino acid sequence set forth in SEQ ID NO: 6, 22, 38, 54, 70, 86, 102, 118, 134, 150, 158, 166, 180, or 196, or encoded by the nucleotide sequence set forth in SEQ ID NO: 5, 21, 37, 53, 69, 85, 101, 117, 133, 149, 157, 165, 179, or 195, and/or a CDRH2 having the amino acid sequence set forth in SEQ ID NO: 8, 24, 40, 56, 72, 88, 104, 120, 136, 152, 160, 168, 182, or 198, or encoded by the nucleic acid sequence set forth in SEQ ID NO: 7, 23, 39, 55, 71, 87, 103, 119, 135, 151, 159, 167, 181, or 197.

In some embodiments, the antibody contains a VH region having an amino acid sequence set forth in SEQ ID NO: 4, 20, 36, 52, 68, 84, 100, 116, 132, 148, 156, 164, 178, 194, 203, 204, 205, 206, 227, 228, 229, 230, 231, or 232, with or without the leader sequence, or a VH region encoded by the nucleotide sequence set forth in SEQ ID NO: 3, 19, 35, 51, 67, 83, 99, 115, 131, 147, 155, 163, 177, or 193, with or without the leader sequence.

In some embodiments, the antibody contains one, two, or three light chain CDR (CDRL) of a light chain variable (VL) region set forth herein, as determined by any known method, such as those described herein. In some embodiments, the antibody contains one or more CDRH of a VL region having an amino acid sequence set forth in SEQ ID NO: 12, 28, 44, 60, 76, 92, 108, 124, 140, 186, 207, 208, 209, 210, 212, 220, 233, 234, 235, 236, or 237, or of a VL region encoded by a nucleotide sequence set forth in SEQ ID NO: 11, 27, 43, 59, 75, 91, 107, 123, 139, 185, 211, or 219 such as the CDRL1, CDRL2, and/or CDRL3 of such a sequence, as determined by any known numbering scheme for identifying CDRs, such as any described herein.

For example, in some embodiments, the antibody contains a CDRL3 having the amino acid sequence set forth in SEQ ID NO: 18, 34, 50, 66, 82, 98, 114, 130, 146, 176, 192, 218, or 226 or encoded by the nucleotide sequence set forth in SEQ ID NO: 17, 33, 49, 65, 81, 97, 113, 129, 145, 175, 191, 217, or 225, a CDRL1 having the amino acid sequence set forth in SEQ ID NO: 14, 30, 46, 62, 78, 94, 110, 126, 142, 172, 188, 214, or 222, or encoded by the nucleotide sequence set forth in SEQ ID NO: 13, 29, 45, 61, 77, 93, 109, 125, 141, 171, 187, 213, or 221, and/or a CDRL2 having the amino acid sequence set forth in SEQ ID NO: 16, 32, 48, 64, 80, 96, 112, 128, 144, 174, 190, 216, or 224, or encoded by the nucleic acid sequence set forth in SEQ ID NO: 15, 31, 47, 63, 79, 95, 111, 127, 143, 175, 189, 215, or 223.

In some embodiments, the antibody contains a VL region having an amino acid sequence set forth in SEQ ID NO: 12, 28, 44, 60, 76, 92, 108, 124, 140, 186, 207, 209, 209, 210, 212, 220, 233, 234, 235, 236, or 237, with or without the leader sequence, or a VL region encoded by the nucleotide sequence set forth in SEQ ID NO: 11, 27, 43, 59, 75, 91, 107, 123, 139, 185, 211, or 219 with or without the leader sequence.

Also among the provided embodiments are antibodies that compete for binding to antigen with antibodies having such variable region(s) and/or CDR sequences. In some embodiments, the provided antibody further includes one or more constant region.

In other embodiments, the antibodies contain VH and/or VL amino acid sequences having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to any of the VH and/or VL set forth herein. In some embodiments, the antibodies contain CDRH 1, 2, and/or 3, and/or CDRL 1, 2, and/or 3 sequences having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99° A identity to any of the CDR sequences set forth herein.

In some embodiments, the antibody has a VH region having a CDRH1 having an amino acid sequence as set forth in SEQ ID NO: 6, a CDRH2 amino acid as set forth in SEQ ID NO: 8, and/or a CDRH3 sequence as set forth in SEQ ID NO: 10, and/or has a VL region having a CDRL1 having an amino acid sequence as set forth in SEQ ID NO: 14, a CDRL2 amino acid as set forth in SEQ ID NO: 16, and/or a CDRL3 sequence as set forth in SEQ ID NO: 18, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 4, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region as set forth in SEQ ID NO: 12, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 22, a CDRH2 amino acid as set forth in SEQ ID NO: 24, and/or a CDRH3 sequence as set forth in SEQ ID NO: 26, and/or has a VL region having a CDRL1 having an amino acid sequence as set forth in SEQ ID NO: 30, a CDRL2 amino acid as set forth in SEQ ID NO: 32, and/or a CDRL3 sequence as set forth in SEQ ID NO: 34, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 20, 203, 204, 205, or 206, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region as set forth in SEQ ID NO: 28, 207, 208, 209, or 210, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 38, a CDRH2 amino acid as set forth in SEQ ID NO: 40, and/or a CDRH3 sequence as set forth in SEQ ID NO: 42, and/or has a VL region having a CDRL1 having an amino acid sequence as set forth in SEQ ID NO: 46, a CDRL2 amino acid as set forth in SEQ ID NO: 48, and/or a CDRL3 sequence as set forth in SEQ ID NO: 50, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 36, 227, 228, 229, 230, 231, or 232 with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region as set forth in SEQ ID NO: 44, 233, 234, 235, 236, or 237, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 54, a CDRH2 amino acid as set forth in SEQ ID NO: 56, and/or a CDRH3 sequence as set forth in SEQ ID NO: 58, and/or has a VL region having a CDRL1 having an amino acid sequence as set forth in SEQ ID NO: 62, a CDRL2 amino acid as set forth in SEQ ID NO: 64, and/or a CDRL3 sequence as set forth in SEQ ID NO: 66, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 52, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region as set forth in SEQ ID NO: 60, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 70, a CDRH2 amino acid as set forth in SEQ ID NO: 72, and/or a CDRH3 sequence as set forth in SEQ ID NO: 74, and/or has a VL region having a CDRL1 having an amino acid sequence as set forth in SEQ ID NO: 78, a CDRL2 amino acid as set forth in SEQ ID NO: 80, and/or a CDRL3 sequence as set forth in SEQ ID NO: 82, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 68, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region as set forth in SEQ ID NO: 76, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 86, a CDRH2 amino acid as set forth in SEQ ID NO: 88, and/or a CDRH3 sequence as set forth in SEQ ID NO: 90, and/or has a VL region having a CDRL1 having an amino acid sequence as set forth in SEQ ID NO: 94, a CDRL2 amino acid as set forth in SEQ ID NO: 96, and/or a CDRL3 sequence as set forth in SEQ ID NO: 98, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 84, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region as set forth in SEQ ID NO: 92, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 102, a CDRH2 amino acid as set forth in SEQ ID NO: 104, and/or a CDRH3 sequence as set forth in SEQ ID NO:106, and/or has a VL region having a CDRL1 having an amino acid sequence as set forth in SEQ ID NO:110, a CDRL2 amino acid as set forth in SEQ ID NO:112, and/or a CDRL3 sequence as set forth in SEQ ID NO: 114, or the sequences have at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 100, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region as set forth in SEQ ID NO: 108, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 118, a CDRH2 amino acid as set forth in SEQ ID NO: 120, and/or a CDRH3 sequence as set forth in SEQ ID NO:122, and/or has a VL region having a CDRL1 having an amino acid sequence as set forth in SEQ ID NO:126, a CDRL2 amino acid as set forth in SEQ ID NO:128, and/or a CDRL3 sequence as set forth in SEQ ID NO: 130, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 116, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region as set forth in SEQ ID NO: 124, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 134, a CDRH2 amino acid as set forth in SEQ ID NO: 136, and/or a CDRH3 sequence as set forth in SEQ ID NO:138, and/or has a VL region having a CDRL1 having an amino acid sequence as set forth in SEQ ID NO:142, a CDRL2 amino acid as set forth in SEQ ID NO:144, and/or a CDRL3 sequence as set forth in SEQ ID NO: 146, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 132, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region as set forth in SEQ ID NO: 140, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 150, a CDRH2 amino acid as set forth in SEQ ID NO: 152, and/or a CDRH3 sequence as set forth in SEQ ID NO:154, and/or has a VL region having a CDRL1 having an amino acid sequence as set forth in SEQ ID NO:214, a CDRL2 amino acid as set forth in SEQ ID NO:216, and/or a CDRL3 sequence as set forth in SEQ ID NO: 218, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 148, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region with an amino acid sequence as set forth in SEQ ID NO: 212, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 158, a CDRH2 amino acid as set forth in SEQ ID NO: 160, and/or a CDRH3 sequence as set forth in SEQ ID NO:162, and/or has a VL region having a CDRL1 having an amino acid sequence as set forth in SEQ ID NO:222, a CDRL2 amino acid as set forth in SEQ ID NO:224, and/or a CDRL3 sequence as set forth in SEQ ID NO: 226, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 156, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region with an amino acid sequence as set forth in SEQ ID NO: 220, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 166, a CDRH2 amino acid as set forth in SEQ ID NO: 168, and/or a CDRH3 sequence as set forth in SEQ ID NO:170, and/or has a VL region having a CDRL1 having an amino acid sequence as set forth in SEQ ID NO: 172, a CDRL2 amino acid as set forth in SEQ ID NO:174, and/or a CDRL3 sequence as set forth in SEQ ID NO: 176, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 164, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region with an amino acid sequence, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 180, a CDRH2 amino acid as set forth in SEQ ID NO: 182, and/or a CDRH3 sequence as set forth in SEQ ID NO:184, and/or has a VL region having a CDRL1 having an amino acid sequence as set forth in SEQ ID NO:188, a CDRL2 amino acid as set forth in SEQ ID NO:190, and/or a CDRL3 sequence as set forth in SEQ ID NO:192, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 178, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region with an amino acid sequence as set forth in SEQ ID NO: 186, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

In some embodiments, the antibody has a VH region having a CDRH1 having an amino acid sequence as set forth in SEQ ID NO: 196, a CDRH2 amino acid as set forth in SEQ ID NO: 198, and/or a CDRH3 sequence as set forth in SEQ ID NO:200, and/or has a VL region having a CDRL1, a CDRL2, and/or a CDRL3 sequence, or has at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such an antibody, or competes for binding to DDR1 with such an antibody.

For example, in some aspects, the antibody has a VH region having the amino acid sequence set forth in SEQ ID NO: 194, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, and a VL region with an amino acid sequence, with or without the leader sequence, or having at or about or at least at or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to such a sequence, or competes for binding to DDR1 with such an antibody.

Included among the provided antibodies are humanized variants of such antibodies, as well as human and chimeric variants. Also among the provided antibodies are those in which modifications have been made to framework residues within VH and/or VL. In some aspects, such framework modifications are made to decrease immunogenicity, for example, by “backmutating” one or more framework residues to the corresponding germline sequence, for example, in an antibody that has undergone somatic mutation and may contain framework residues differing from the germline sequence from which the antibody is derived. In some aspects, the antibodies have modifications in one or more framework or even CDR residues to remove T-cell epitopes and reduce potential immunogenicity, or within the Fc region, for example, to alter one or more functional properties of the antibody, such as serum half-life, complement fixation, Fc receptor binding, and/or antigen-dependent cellular cytotoxicity, and/or that are chemically modified, or modified to alter glycosylation, using any of a number of known methods.

Various methods for the preparation of antibodies, such as monoclonal antibodies, are well known in the art and can be used to produce the provided antibodies. For example, antibodies can be prepared by immunizing a suitable mammalian host using a DDR1 protein, peptide, or fragment, in isolated or immunoconjugated form (Antibodies: A Laboratory Manual, CSH Press, Eds., Harlow, and Lane (1988); Harlow, Antibodies, Cold Spring Harbor Press, NY (1989)), or using a fusion protein, such as a DDR1-GST or -Fc fusion protein. In some embodiments, the antibody is generated using a protein or peptide with an amino acid sequence containing all or part of SEQ ID NO: 1 as an immunogen. In some embodiments, immortalized cell lines secreting a desired monoclonal antibody are prepared using standard hybridoma technology, such as that described of Kohler and Milstein or modifications thereof that immortalize antibody-producing B cells. Immortalized cell lines secreting the antibodies may be screened by immunoassay or other known technique with DDR, e.g., DDR1 proteins or peptides. In some examples, cells are expanded; antibodies may be produced either from in vitro cultures or from ascites fluid.

The antibodies or fragments of the invention can also be produced by recombinant means, including known methods to generate chimeric antibodies and complementarity-determining region (CDR) grafted antibodies of multiple species origin, such as humanized antibodies. See for example, Jones et al., 1986, Nature 321: 522-525; Riechmann et al., 1988, Nature 332: 323-327; Verhoeyen et al., 1988, Science 239: 1534-1536). See also, Carter et al., 1993, Proc. Natl. Acad. Sci. USA 89: 4285 and Sims et al., 1993, J. Immunol. 151: 2296.

Fully human antibodies may be produced using any of a number of known techniques, such as use of transgenic mice engineered to express human immunoglobulin genes, such as the Xenomouse (Amgen Fremont, Inc.), those described by U.S. Pat. No. 6,657,103, U.S. Pat. Nos. 5,569,825; 5,625,126; 5,633,425; 5,661,016; and 5,545,806, Mendez, et. al. Nature Genetics, 15: 146-156 (1998), Kellerman, S. A. & Green, L. L., Curr. Opin. Biotechnol 13, 593-597 (2002), Lonberg, et al. (1994) Nature 368(6474): 856-859)), Tomizuka et al. (2000) Proc. Natl. Acad. Sci. USA 97:722-727, and PCT Publication WO 02/43478 to Tomizuka, et al., using SCID mice reconstituted with human cells (see U.S. Pat. Nos. 5,476,996 and 5,698,767 to Wilson et al.), and using phage display methods for screening libraries of human immunoglobulin genes, including for example, those described in U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al. Other techniques include those described by U.S. Pat. Nos. 6,586,251, 6,596,541, 7,105,348, 6,528,313, 6,638,768, and 6,528,314.

### Antibody Assays

Reactivity, binding, specificity, affinity, potency, interactions, and other properties of the antibodies with DDR1 protein can be established by a number of well-known techniques. The term “reactivity” relates to the interaction or binding of an antibody with its cognate antigen. The term “specificity” or sometime “selectivity” relates the relative strength of antibody affinity for the desired target, as opposed to the universe of all other possible targets. Typically specificity is reported as fold-difference in Kd. For example, if an antibody binds to protein X, but not to protein Y, then the exact specificity cannot be calculated, except to say that it is greater than the assay range. For the present application, disclosed antibodies are consider “specific” if binding to DDR2 falls below certain parameters in the various assays discussed herein or otherwise known to those of ordinary skill in the art.

Generally, the term “affinity” refers to the strength of the physical association (binding) between two molecules. Affinity is typically reported as an equilibrium constant (Kd) measurement (units=molar concentration). The tighter the binding, the lower the Kd value. Terms such as “adequate,” “good,” or “strong” affinity, are relative in nature. In an in vitro setting, antibodies with monomeric antigen affinity <1 nM may be considered to possess “strong” binding affinity. “Potency” refers to the strength of the inhibitory activity of an antibody as measured in a specific assay. Typically, potency is reported as an EC₅₀ measurement (units=molar concentration). The stronger the potency, typically the lower the EC₅₀. Like affinity, defining “adequate,” “good,” or “strong” potency, may be problematic. For in vitro purposes only, we consider antibodies with potency EC₅₀<1 nM to be “strong.” For example, to assess binding or specificity, or affinity, the suitable techniques include, but are not limited to Western blot, immunoprecipitation, ELISA, surface plasmon resonance using a Biacore instrument, and flow cytometry, using, as appropriate, DDR1 proteins, peptides, and fragments thereof, and/or cells expressing the same. As various techniques measure different antigen presentation and surface interactions between antigens and antibodies, one skilled in the art understands that the values from different technologies likely result in different values for the binding, specificity, or affinity constant. In some embodiments, the antibodies are labeled with detectable markers or conjugated to secondary antibodies that are labeled with a detectable marker, such as a radioisotope, a fluorescent compound, a bioluminescent compound, chemiluminescent compound, a metal chelator or an enzyme.

The ability of DDR1 and its binding partners to mediate interactions between cells is used in one embodiment to evaluate an anti-DDR1 antibody for its ability to block the formation of multi-cell clusters of cells that express DDR1 either endogenously or recombinantly. For example, a DDR1 expressing tumor cell line can be analyzed for the ability to form multi-cell clusters when treated with an anti-DDR1 antibody. The tumor cells can be transformed cell lines, primary cancer cells isolated from a cancer tissue of a subject, or cancer cell in a subject in vivo. In one embodiment, A431 cells (a human epidermoid carcinoma cell line) are plated as a single-cell suspension and cultured in media containing a collagen/matrigel mixture. An anti-DDR1 antibody is added and incubated. In one aspect, multi-cell cluster formation in untreated cells and cells treated with the anti-DDR1 antibody is compared by visual observation. In one embodiment, cells treated with the anti-DDR1 antibody exhibit impaired cluster formation with smaller groupings, while untreated cells form large multi-cell clusters. Without being bound any theory, the multi-cell cluster formation assay can be automated and has high-throughput potential.

In addition, point-test assays can be used to determine the normalized percent inhibition (NPI). Signals from the vehicle-treated cells (i.e. negative control) are defined as 0% inhibition and signals from the cells treated with the anti-DDR1 polyclonal antibody (i.e. positive control) are defined as 100% inhibition. Where quantitative data is not available, qualitative assessment of cluster formation can be used to indicate either full inhibition (i.e. equivalent to 100% NPI) or no inhibition (i.e. equivalent to 0% NPI). By way of example, when NPI at the point of antibodies at 66 nm is less than about 40%, there is no inhibition of cluster formation. In one embodiment, the anti-DDR1 antibody inhibits the cluster formation.

In another embodiment, an anti-DDR1 antibody is assessed for its ability to alter the collagen-mediated subcellular relocalization of DDR1. This embodiment takes advantage of the observation that, in certain tumor cells, DDR1 is primarily localized to the outer cell membrane in cells grown on plastic dishes without stimulation. Upon collagen stimulation, the localization of DDR1 is altered. For example, the collagen-mediated relocalization of DDR1 can be inhibited by an anti-DDR1 antibody. In some embodiments, the anti-DDR1 antibodies inhibit the relocation of DDR1 protein that is mediated or stimulated by collagen. Suitable the tumor cells for use with the assay include transformed cell lines, primary cancer cells isolated from a cancer tissue of a subject, or cancer cell in a subject in vivo that express DDR-1. In one embodiment, HCT-116 tumor cells (a human colorectal carcinoma cell line) are used in the assay, which measures the impact of an anti-DDR1 on receptor relocalization after stimulation. Without being bound any theory, the DDR1 relocalization assay can be automated and has high-throughput potential.

In another assay, an anti-DDR1 antibody is evaluated by a cell-based NFκB luciferase reporter assay. Any suitable NFκB luciferase reporter assay can be used. In one such assay, a cell line that recombinantly expresses is prepared, by transfecting the cells with the NFκB reporter construct, and assayed in the presence or absence of an anti-DDR1 antibody at various concentrations. After stimulation, cells are analyzed for luciferase activity.

In another assay, an anti-DDR1 antibody is evaluated by measuring by measuring phosphorylation of DDR1 at tyrosine 513 in response to collagen stimulation, and in the presence or absence of the candidate antibody. For example, the engineered cells that express DDR1 with a ProLink™ Tag (PK) and enzyme acceptor (EA) tagged-SHC1 adaptor protein (DiscoveRx Corporation) are prepared. The cells are treated with Collagen II, which initiates DDR1 phosphorylation and recruitment of SHC-1-EA to the receptor. This interaction leads to complementation of the two β-galactosidase enzyme fragments (EA and PK) to make active enzyme, which upon addition of DiscoveRx substrate solution hydrolyzes the substrate to generate chemiluminescent signal proportional to the tyrosine 513 DDR1 phosphorylation. Chemiluminescence can be measured using a BioTek Synergy plate reader in the presence and absence of a candidate antibody screened to identify those that inhibit phosphorylation of DDR1 at tyrosine 513. EC₅₀ values generated by the methods described above (NFκB reporter, cluster formation and phosphorylation assays) can be used to evaluate the potency of an anti-DDR1 antibody.

In some embodiments, the anti-DDR1 antibodies exhibit one or more properties of inhibiting cluster formation, specific binding to DDR1, inhibits the relocation of DDR1 relocalization that is mediated or stimulated by collagen, high binding affinity to DDR1, and being inhibitory antibodies.

Therapeutic, Detection, and Diagnostic Methods

Also provided are methods of using the antibodies, including in detection, diagnostic, and therapeutic methods, and uses of the antibodies in such methods. For example, provided are methods and uses of the antibodies to treat, diagnose, or detect a disease or condition associated with DDR1. Such DDR1-associated diseases and conditions include, but are not limited to cancer, e.g., breast cancer, lung cancer, ovarian cancer, brain cancer, esophageal cancer, metastasis, angiogenesis, tumor invasion and/or progression, diseases associated with cell proliferation, cell invasion, and/or deregulation of extracellular matrix production, and/or fibrosis, and inflammatory and autoimmune diseases, such as but not limited to glomerulonephritis, rheumatoid arthritis. See, e.g., Vogel et al., Molecular Cell, Vol. 1, 13-23, December, 1997; Vogel et al., The FASEB Journal, Vol. 13, Supplement, s77-s82, 1999; Yoshimura et al., Immunologic Research, 31(3): 219-29.

Also provided are pharmaceutical compositions for use in connection with such methods, such as those containing any of the antibodies described herein. Compositions can be suitable for administration locally or systemically by any suitable route. The antibodies of the present invention or the pharmaceutical compositions comprising the same may be combined with one or more other therapeutic agents.

The therapeutic agent may be a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, or an therapeutic antibody.

In another embodiment, the antibodies contemplated herein are combined with one or more chemotherapeutic agents.

Chemotherapeutic agents may be categorized by their mechanism of action into, for example, the following groups: anti-metabolites/anti-cancer agents, such as pyrimidine analogs floxuridine, capecitabine, and cytarabine) and purine analogs, folate antagonists and related inhibitors antiprobliferative/antimitotic agents including natural products such as vinca alkaloid (vinblastine, vincristine, and microtubule such as taxane (paclitaxel, docetaxel), vinblastin, nocodazole, epothilones and navelbine, epidipodophyllotoxins (etoposide, teniposide), DNA damaging agents (actinomycin, amsacrine, busulfan, carboplatin, chlorambucil, cisplatin, cyclophosphamide, Cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, triethylenethiophosphoramide and etoposide; antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards cyclophosphamide and analogs, melphalan, chlorambucil), and (hexamethylmelamine and thiotepa), alkyl nitrosoureas (BCNU) and analogs, streptozocin), trazenes-dacarbazinine (DTIC); antiproliferative/antimitotic antimetabolites such as folic acid analogs (methotrexate); platinum coordination complexes (cisplatin, oxiloplatinim, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (breveldin); immunosuppressives tacrolimus sirolimus azathioprine, mycophenolate; compounds (TNP-470, genistein) and growth factor inhibitors (vascular endothelial growth factor inhibitors, fibroblast growth factor inhibitors); angiotensin receptor blocker, nitric oxide donors; anti-sense oligonucleotides; antibodies (trastuzumab, rituximab); cell cycle inhibitors and differentiation inducers (tretinoin); inhibitors, topoisomerase inhibitors (doxorubicin (adriamycin), daunorubicin, dactinomycin, eniposide, epirubicin, etoposide, idarubicin, irinotecan and mitoxantrone, topotecan, irinotecan), corticosteroids (cortisone, dexamethasone, hydrocortisone, methylpednisolone, prednisone, and prenisolone); growth factor signal transduction kinase inhibitors; dysfunction inducers, toxins such as Cholera toxin, ricin, Pseudomonas exotoxin, Bordetella pertussis adenylate cyclase toxin, or diphtheria toxin, and caspase activators; and chromatin.

As used herein the term “chemotherapeutic agent” or “chemotherapeutic” (or “chemotherapy,” in the case of treatment with a chemotherapeutic agent) is meant to encompass any non-proteinaceous (i.e, non-peptidic) chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammaII and calicheamicin phiI1, see, e.g., Agnew, Chem. Intl. Ed. Engl, 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (TAXOL®, Bristol Meyers Squibb Oncology, Princeton, N.J.) and docetaxel (TAXOTERE®, Rhone-Poulenc Rorer, Antony, France); chlorambucil; gemcitabine (Gemzar®); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine®); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston®); inhibitors of the enzyme aromatase, which regulates estrogen production in the adrenal glands, such as, for example, 4(5)-imidazoles, aminoglutethimide, megestrol acetate (Megace®), exemestane, formestane, fadrozole, vorozole (Rivisor®), letrozole (Femara®), and anastrozole (Arimidex®); and anti-androgens such as flutamide, nilutamide, bicalutamide, leuprohde, and goserelin; and pharmaceutically acceptable salts, acids or derivatives of any of the above.

In another embodiment, the antibodies contemplated herein are combined with one or more antiangiogenic agents. Illustrative examples of anti-angiogenic agents include, but are not limited to, retinoid acid and derivatives thereof, 2-methoxyestradiol, ANGIOSTATIN®, ENDOSTATIN®, suramin, squalamine, tissue inhibitor of metalloproteinase-1, tissue inhibitor of metalloproternase-2, plasminogen activator inhibitor-1, plasminogen activator inbibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,I-3,4-dehydroproline, thiaproline, .alpha.-dipyridyl, .beta-aminopropionitrile fumarate, 4-propyl-5-(4-pyridinyl)-2(3 h)-oxazolone; methotrexate, mitoxantrone, heparin, interferons, 2 macroglobulin-serum, chimp-3, chymostatin, .beta.-cyclodextrin tetradecasulfate, eponemycin; fumagillin, gold sodium thiomalate, d-penicillamine (CDPT), beta.-1-anticollagenase-serum, alpba.2-antiplasmin, bisantrene, lobenzarit disodium, n-2-carboxyphenyl-4-chloroanthronilic acid disodium or “CCA”, thalidomide; angiostatic steroid, cargboxynaminolmidazole; metalloproteinase inhibitors such as BB94. Other anti-angiogenesis agents include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

In another embodiment, the antibodies contemplated herein are combined with one or more anti-fibrotic agents. Exemplary anti-fibrotic agents include, but are not limited to the compounds such as .beta-aminoproprionitrile (BAPN), as well as the compounds disclosed in U.S. Pat. No. 4,965,288 to Palfreyman, et al., issued Oct. 23, 1990, entitled “Inhibitors of lysyl oxidase,” relating to inhibitors of lysyl oxidase and their use in the treatment of diseases and conditions associated with the abnormal deposition of collagen; U.S. Pat. No. 4,997,854 to Kagan, et al., issued Mar. 5, 1991, entitled “Anti-fibrotic agents and methods for inhibiting the activity of lysyl oxidase in situ using adjacently positioned diamine analogue substrate,” relating to compounds which inhibit LOX for the treatment of various pathological fibrotic states, which are herein incorporated by reference. Further exemplary inhibitors are described in U.S. Pat. No. 4,943,593 to Palfreyman, et al., issued Jul. 24, 1990, entitled “Inhibitors of lysyl oxidase,” relating to compounds such as 2-isobutyl-3-fluoro-, chloro-, or bromo-allylamine; as well as, e.g., U.S. Pat. No. 5,021,456; U.S. Pat. No. 5,5059,714; U.S. Pat. No. 5,120,764; U.S. Pat. No. 5,182,297; U.S. Pat. No. 5,252,608 (relating to 2-(1-naphthyloxymemyl)-3-fluoroallylamine); and U.S. Patent Application No. 2004/0248871, which are herein incorporated by reference. Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives, semicarbazide, and urea derivatives, aminonitriles, such as beta-aminopropionitrile (BAPN), or 2-nitroethylamine, unsaturated or saturated haloamines, such as 2-bromo-ethylamine, 2-chloroethylamine, 2-trifluoroethylamine, 3-bromopropylamine, p-halobenzylamines, selenohomocysteine lactone. In another embodiment, the anti-fibrotic agents are copper chelating agents, penetrating or not penetrating the cells. Additional exemplary compounds include indirect inhibitors such compounds blocking the aldehyde derivatives originating from the oxidative deamination of the lysyl and hydroxylysyl residues by the lysyl oxidases, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-342-aminoethyl)dithio)butanoic acid, sodium-4-((p-1-dimethyl-2-amino-2-carboxyethyl)dithio)butane sulphanate, 2-acetamidoethyl-2-acetamidoethanethiol sulphanate, sodium-4-mercaptobutanesulphinate trihydrate.

In further embodiments, the antibodies contemplated herein are combined with one or more other antibodies.

Example of additional antibodies suitable for combination with the inventive antibodies contemplated herein, include but are not limited to, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomabn, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49 and 3F8.

In another embodiment, the antibodies contemplated herein are combined with one or more anti-MMP9 antibodies or anti-DDR1 antibodies.

In one embodiment, the one or more therapeutic agent may be an inhibitor to phosphatidylinositide 3-kinases (PI3K) such as PI3Kγ, PI3Kα, PI3Kβ, or PI3Kδ, spleen tyrosine kinase, lysyl oxidase-like protein such as LOXL1, LOXL2, LOXL3, LOXL4, or LOXL5, matrix metalloprotease (MMP) such as any one of MMP 2, 3, 7, 8, or 9, adenosine A2B receptor, isocitrate dehydrogenase (IDH) such as IDH1, Janus kinases (JAK) such as JAK1, JAK2, or JAK3, bruton's tyrosine kinase, apoptosis signal-regulating kinase, serine/threonine kinase Tpl2, or any combination thereof.

In other embodiments, the one or more therapeutic agent is: a JAK inhibitor, including but not limited to, momelotinib, Ruxolitinib (INCB018424, Incyte Pharmaceuticals/Novartis), SAR302503 (Sanofi), pacritinib (Cell Therapeutics), INCB039110 (Incyte), LY2784544 (Eli Lilly), BMS911543 (Bristol-Myers Squibb), NS018 (Nippon Shinyaku); a myelofibrosis inhibiting agent, including but not limited to, hedgehog inhibitors (saridegib from Infinity), histone deacetylase (HDAC) inhibitors (pracinostat from MEI Pharm, panobinostat from Novartis), tyrosine kinase inhibitor (lestaurtinib from Teva); a DDR1 inhibitor, including but not limited to, those disclosed in US2009/0142345 (Takeda Pharmaceutical), US2011/0287011 (OncoMed Pharmaceuticals), WO2013027802 (Chugai Pharmaceutical), WO2013034933 (Imperial Innovations), or those developed by Kolltan Pharmaceuticals; an MMP9 inhibitor, a LOXL2 inhibitor, an ASK1 inhibitor, a PI3Kδ inhibitor, including but not limited to, Idelalisib, PI3K II, TGR-1202 (TG Therap.), AMG-319 (Amgen), GSK2269557 (GSK), X-339 (Xcovery), X-414 (Xcovery), RP5090 (Incozen Therap.), KAR4141 (Karus Therap.), XL499 (Merck), OXY111A (NormOxys), IPI-145 (Infinity/Takeda), IPI-443 (Infinity); a PI3Kβ inhibitor, including but not limited to, GSK2636771 (GSK), BAY 10824391 (Bayer); a PI3Kα inhibitor, including but not limited to, Buparlisib (Novartis), BAY 80-6946 (Bayer), BYL719 (Novartis), PX-866 (Oncothyreon), RG7604 (Roche), MLN1117 (Takeda), WX-037 (Wilex/UCB), AEZS-129 (Aeterna Zentaris), PA799 (Chugai); a PI3Kγ inhibitor, including but not limited to, ZSTK474 (Zenyaku Koyo); a BTK inhibitor, including but not limited to, Ibrutinib (Pharmacyclics/J&J), HM71224 (Hanmi), ONO-4059 (Ono), CC-292 (Celgene); a SYK inhibitor, including but not limited to, R406, fostamatinib, BAY-61-3606, NVP-QAB 205 AA, R112, or R343; a BRD4 inhibitor, an IDH1 inhibitor, a TPL2 inhibitor, or an A2b inhibitor.

In some embodiments, the one or more therapeutic agents is a PI3K inhibitor such as Idelalisib, a JAK inhibitor such as Momelotinib, a LOXL2 inhibitor such as Simtuzumab, an anti-MMP9 antibody, or an anti-DDR1 antibody or a combination thereof.

In general, the antibodies are administered in a therapeutically effective amount, e.g., in an amount to effect treatment of a particular disease or condition, such as to effect a reduction or elimination of a symptom thereof, and/or an amount effective to inhibit DDR1 activity.

The selected dosage regimen will depend upon a variety of factors, which may include the activity of the antibody, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

A clinician having ordinary skill in the art can readily determine and prescribe the effective amount (ED₅₀) of the pharmaceutical composition required. For example, the physician or veterinarian can start doses of the compounds of the invention employed in the pharmaceutical composition at levels lower than that required in order to achieve the desired therapeutic effect and gradually increase the dosage until the desired effect is achieved.

In some cases, the methods of treatment include parenteral administration, e.g., intravenous, intra-arterial, intramuscular, or subcutaneous administration, or oral administration of the antibody or composition containing the same. The antibodies may also be administered locally.

If needed, for treatments, methods can further include additional therapies, such as in the case of cancer, surgical removal of the cancer and/or administration of an anti-cancer agent or treatment in addition to administration of the antibody. In some cases, administration of such an additional therapy can be concurrent with administration of the compositions or antibodies disclosed herein.

In some embodiments, the treatment methods include steps for monitoring treatment, including for monitoring efficacy or activity and/or detecting or measuring the presence, absence, levels, and/or expression of markers, including DDR1 and/or other markers of the disease or condition of interest.

The present disclosure also contemplates methods of detection using the provided antibodies, such as methods of detecting DDR1 and associated disease or condition, in a subject. Thus, the provided methods include diagnostic, prognostic, detection, and monitoring methods.

In some embodiments, samples (e.g., test biological samples) from a subject (e.g., a human suspected of having or known to have a disease or condition associated with DDR1 expression or activity) are analyzed for the presence, absence, expression, and/or levels of DDR1. For example, such samples can be collected and analyzed by detecting the presence or absence of binding of an antibody that specifically binds to DDR1, such as any of the provided antibodies, to substance (e.g., protein) in the sample. In some examples, the methods further include comparing the amount of binding detected to an amount of binding to a control sample, or comparing the detected level or activity of DDR1 to a control level or activity of DDR1. In some cases, the methods indicate the presence, absence, or severity of a disease or condition as described herein. In other embodiments, samples from a subject are analyzed for the presence, absence, and/or levels of phosphate moieties on DDR1 to determine its activities. By way of example, the phosphate moieties on DDR1 may be assessed using the levels of tyrosine phosphorylation on the DDR1 protein.

This analysis can be performed prior to the initiation of treatment using an antibody as described herein, or can be done as part of monitoring of progress of treatment. In some embodiments, provided are methods of treatment, carried out by performing the detection assays and initiating, altering, or discontinuing treatment of the subject, for example, based on the results of the diagnostic assay. Such diagnostic analysis can be performed using any sample, including but not limited to tissue, cells isolated from such tissues, and the like. In some cases, the methods are performed on liquid samples, such as blood, plasma, serum, whole blood, saliva, urine, or semen. Tissue samples include, for example, formalin-fixed or frozen tissue sections.

Any suitable method for detection and analysis of DDR1 can be employed. Various diagnostic assay techniques known in the art can be adapted for such purpose, such as competitive binding assays, direct or indirect sandwich assays and immunoprecipitation assays conducted in either heterogeneous or homogeneous phases.

Antibodies for use in detection methods can be labeled with a detectable moiety. The detectable moiety directly or indirectly produces a detectable signal. For example, the detectable moiety can be any of those described herein such as, for example, a radioisotope, such as ³H, ¹⁴C, ³²P, ³⁵S, or ¹²⁵I, a fluorescent or chemiluminescent compound, such as fluorescein isothiocyanate (FITC), Texas red, cyanin, photocyan, rhodamine, or luciferin, or an enzyme, such as alkaline phosphatase, β-galactosidase or horseradish peroxidase.

Detection can be accomplished by contacting a sample under conditions suitable for antibody binding to DDR1, and assessing the presence (e.g., level) or absence of antibody-protein (e.g., DDR1) complexes. A level of DDR1 in the sample in comparison with a level of a reference sample can indicate the presence of a disease or condition associated with DDR1. The reference sample can be a sample taken from the subject at an earlier time point or a sample from another individual.

Various aspects of the invention are further described and illustrated by way of the examples which follow, none of which are intended to limit the scope of the invention.

### Example 1

### Generation of Anti-DDR1Antibodies

A DDR1 fusion protein (DDR1ECD-Fc (purchased from R&D Systems, Catalog #2396-DR)) was used as an immunogen to generate anti-DDR1 antibodies. The DDR1ECD-Fc contained a human DDR1 extracellular domain (ECD) region from Asp21-Thr416 of the human DDR1 amino acid sequence and a IEFRMD linker, fused to human IgG1 (Pro100-Lys330).

Mice were immunized with the DDR1ECD-Fc immunogen or DDR1ECD-6Xhis, with Ribi adjuvant, using a PolyExpress protocol from Antibody Solutions. B cells from lymph nodes of immunized mice were fused with mouse myeloma cells to generate a hybridoma library, from which individual hybridomas were derived.

Ten (10) monoclonal antibodies produced by individual hybridomas derived from the library were designated AB2004, AB2009, AB2026, AB2039, AB2031, AB2041, AB2074, AB2078, AB2079, AB2092. The nucleotide and amino acid sequences for the variable heavy (VH) chain and variable light (VL) chain were determined for each antibody and are listed in Table 1, with the leader sequence set forth in italics and complementarity determining region (CDR) sequences set forth in bold. Individual CDR1, 2, and 3 sequences also are listed for each VH and VL sequence.

TABLE 1
Anti-DDR1 Monoclonal Antibodies

| | |
|---|---|
| | SEQ |
| | ID |
| Sequences | NO |

AB2004

| | | | |
|---|---|---|---|
| VH | Full | ATGAGAGTGCTGATTCTTTTGTGCCTGTTCACAGCCTTTCCTGGTAT | 3 |
| | Length | CCTGTCTGATGTACATCTTCAGGAGTCAGGACCTGACCTGGTGA | |
| | (leader | AACCTTCTCAGTCACTTTCACTCACCTGCACTGTCACTGACTAC | |
| | sequence | TCCATCACCAGTGGTTATGGCTGGCACTGGATCCGGCAGTTT | |
| | in italics) | CCAGGAAACAAACTGGAATGGATGGGCTACATACACTACGGT | |
| | | GGTAGCACTAAGTACAATTCATCTCTCAAAAGTCGCATCTCT | |
| | | ATCACTCGAGACACATCCAAGAACCAGTTCTTCCTGCAATTGA | |
| | | AATCTGTGACTACTGATGACACAGGCACATATTACTGTGCAAC | |
| | | CTCTGGTAACTACGGATTTGCTTACTGGGGCCAAGGGACTCT | |
| | | GGTCACTGTCTCTGCA | |
| | | MRVLILLCLFTAFPGILSDVHLQESGPDLVKPSQSLSLTCTVTDYSIT | 4 |
| | | SGYGWHWIRQFPGNKLEWMGYIHYGGSTKYNSSLKSRISITRDT | |
| | | SKNQFFLQLKSVTTDDTGTYYCATSGNYGFAYWGQGTLVTVSA | |
| | CDRH1 | GACTACTCCATCACCAGTGGTTATGGCTGGCAC | 5 |
| | | DYSITSGYGWH | 6 |
| | CDRH2 | TACATACACTACGGTGGTAGCACTAAGTACAATTCATCTCTCA | 7 |
| | | AAAGT | |
| | | YIHYGGSTKYNSSLKS | 8 |
| | CDRH3 | TCTGGTAACTACGGATTTGCTTAC | 9 |
| | | SGNYGFAY | 10 |
| VL | Full | ATGGAGACAGACACACTCCTGCTATGGGTGCTGCTGCTCTGGGTTC | 11 |
| | Length | CAGGTTCCACAGGTGACATTGTGCTGACCCAATCTCCAACTTCT | |
| | (leader | TTGGCTGTGTCTCTTGGGCAGAGGGCCACCATGTCCTGCAGAG | |
| | sequence | CCAGTGAAAGTGTTGATGATTATGGCACTAGTTTTATGCAC | |
| | in italics) | TGGTACCAGCAGAAACCAGGACAGCCACCCAAACTCCTCATCT | |
| | | ATCGTGCATCCAACCTAGAATCTGGGATCCCTGCCAGGTTCA | |
| | | GTGGCAGTGGGTCTAGGGCAGACTTCACCCTCAGCATTAATCC | |
| | | TGTGGAGCCTGATGATGTTGCAACCTATTACTGTCAGCAAAGT | |
| | | AATGAGGATCCATTCACGTTCGGCTCGGGGACAAAGTTGGAA | |
| | | ATAAAACGG | |
| | | METDTLLLWVLLLWVPGSTGDIVLTQSPTSLAVSLGQRATMSCRAS | 12 |
| | | ESVDDYGTSFMHWYQQKPGQPPKLLIYRASNLESGIPARFSGSGS | |
| | | RADFTLSINPVEPDDVATYYCQQSNEDPFTFGSGTKLEIKR | |
| | CDRL1 | AGAGCCAGTGAAAGTGTTGATGATTATGGCACTAGTTTTATGC | 13 |
| | | AC | |
| | | RASESVDDYGTSFMH | 14 |
| | CDRL2 | CGTGCATCCAACCTAGAATCT | 15 |
| | | RASNLES | 16 |
| | CDRL3 | CAGCAAAGTAATGAGGATCCATTCACG | 17 |
| | | QQSNEDPFT | 18 |

AB2009, AB2026, AB2039, AB2031

| | | | |
|---|---|---|---|
| VH | Full | ATGAACTTAGGGCTCAGCTTCATTTTCCTTGCCCTTATTTTAAAAGG | 19 |
| | Length | TGTCCAGTGTGAGGTGCAGCTGGTGGAGTCTGGGGGAGGCTTA | |
| | (leader | GTGCAGCCTGGAGGGTCCCTGAAATTCTCCTGTGCAGCCTCTG | |
| | sequence | GATTCACTTTCAGTAGCTATGGCATGTCTTGGGTTCGCCAGA | |
| | in Italics) | CTCCAGACAAGAGGCTGGAGTTGGTCGCAATTATTAATAGTC | |
| | | ATGGTTTTAGCACCTATTATCCTGACAGTGTGAAGGGCCGA | |
| | | TTCACCATCTCCAGAGACAATGCCAAGAACACCCTGTACCTGC | |
| | | AAATGAGCAGTCTTAAGTCTGAGGACACAGCCATATATTACTG | |
| | | TGCAAGAGGGGGGTATGGTAACTACGGGATGGACTACTGG | |
| | | GGTCAAGGAACCTCAGTCACCGTCTCCTCA | |
| | | MNLGLSFIFLALILKGVQCEVQLVESGGGLVQPGGSLKFSCAASGF | 20 |
| | | TFSSYGMSWVRQTPDKRLELVAIINSHGFSTYYPDSVKGRFTISR | |
| | | DNAKNTLYLQMSSLKSEDTAIYYCARGGYGNYGMDYWGQGTS | |
| | | VTVSS | |
| | CDRH1 | GGATTCACTTTCAGTAGCTATGGCATGTCT | 21 |
| | | GFTFSSYGMS | 22 |
| | CDRH2 | ATTATTAATAGTCATGGTTTTAGCACCTATTATCCTGACAGTGT | 23 |
| | | GAAGGGC | |
| | | IINSHGFSTYYPDSVKG | 24 |
| | CDRH3 | GGGGGGTATGGTAACTACGGGATGGACTAC | 25 |
| | | GGYGNYGMDY | 26 |
| VL | Full | ATGGAGACAGACACACTCCTGCTATGGGTGCTGCTGCTCTGGGTTC | 27 |
| | Length | CAGGTTCCACAGGTGACATTGTGCTGACCCAATCTCCAGCTTCT | |
| | (leader | TTGGCTGTGTCTCTAGGGCAGAGGGCCACCATATCCTGCAGAG | |
| | sequence | CCAGTGAAAGTGTTGATAGTTATGGCAATAGTTTTATGCAC | |
| | in italics) | TGGTACCAGCAGAAAGTTGGACAGCCACCCAAACTCCTCATCT | |
| | | TTCGTGCATCCAATATAGAATCTGGGATCCCTGCCAGGTTCA | |
| | | GTGGCAGTGGGTCTAGGACAGACTTCACCCTCACCATTAATTC | |
| | | TGTGGAGGCTGATGATGTTGCAACCTATTACTGTCAGCAAAGT | |
| | | AATGAGGCTCCGTACACGTTCGGAGGGGGGACCAAGCTGGA | |
| | | AATAAAACGG | |
| | | METDTLLLWVLLLWVPGSTGDIVLTQSPASLAVSLGQRATISCRA | 28 |
| | | SESVDSYGNSFMHWYQQKVGQPPKLLIFRASNIESGIPARFSGSG | |
| | | SRTDFTLTINSVEADDVATYYCQQSNEAPYTFGGGTKLEIKR | |
| | CDRL1 | AGAGCCAGTGAAAGTGTTGATAGTTATGGCAATAGTTTTATGC | 29 |
| | | AC | |
| | | RASESVDSYGNSFMH | 30 |
| | CDRL2 | CGTGCATCCAATATAGAATCT | 31 |
| | | RASNIES | 32 |
| | CDRL3 | CAGCAAAGTAATGAGGCTCCGTACACG | 33 |
| | | QQSNEAPYT | 34 |

AB2041

| | | | |
|---|---|---|---|
| VH | Full | ATGGAAAGGCACTGGATCTTTCTCTTCCTGTTTTCAGTAACTGCAGG | 35 |
| | Length | TGTCCACTCCCAGGTCCAGCTTCACCAGTCTGGGTCTGAACTGG | |
| | (leader | CAAAACCTGGGGCCTCAGTGAAGATGTCCTGCAAGGCTTCTGG | |
| | sequence | CTACATCTTTACTACCTACTGGATACACTGGTTAAAACAGAG | |
| | in italics) | GCCTGGACAGGGTCTGGAATGGATTGGATTCATTAATCCTGA | |
| | | CACTGATTATACTGAATACAATCAGAAGTTCAAGGACAAGG | |
| | | CCACATTGACTGCAGACAAATCCTCCAACACAGCCTACGTGCA | |
| | | ACTGAGCAGCCTGACATCTGAGGACTCTGCAGTCTATTACTGT | |
| | | GCGAGAGGAAACTATGGTTACGACGGTCTTGTTTACTGGGG | |
| | | CCTAGGGACTCTGGTCACTGTCTCTGCA | |
| | | MERHWIFLFLFSVTAGVHSQVQLHQSGSELAKPGASVKMSCKASG | 36 |
| | | YIFTTYWIHWLKQRPGQGLEWIGFINPDTDYTEYNQKFKDKAT | |
| | | LTADKSSNTAYVQLSSLTSEDSAVYYCARGNYGYDGLVYWGLG | |
| | | TLVTVSA | |
| | CDRH1 | GGCTACATCTTTACTACCTACTGGATACAC | 37 |
| | | GYIFTTYWIH | 38 |
| | CDRH2 | TTCATTAATCCTGACACTGATTATACTGAATACAATCAGAAGTT | 39 |
| | | CAAGGAC | |
| | | FINPDTDYTEYNQKFKD | 40 |
| | CDRH3 | GGAAACTATGGTTACGACGGTCTTGTTTAC | 41 |
| | | GNYGYDGLVY | 42 |
| VL | Full | ATGAAGTCACAGACCCAGGTCTTCGTATTTCTACTGCTCTGTGTGTC | 43 |
| | Length | TGGTGCTCATGGGAGTATTGTGATGACCCAGACTCCCAAATTCC | |
| | (leader | TGCTTGTATCAGCAGGAGACAGGGTTACCATAACCTGCAAGGC | |
| | sequence | CAGTCAGAGTGTGAGTACTGATGTAGCTTGGTACCAACAGA | |
| | in Italics) | AGCCAGGGCAGTCTCCTAAACTGCTGATATACTATGCATCCAA | |
| | | TCGCTACACTGGAGTCCCTGATCGCTTCACTGGCAGTGGATAT | |
| | | GGGACGGATTTCACTTTCACCATCAGCACTGTGCAGGCTGAAG | |
| | | ACCTGGCAGTTTATTTCTGTCAGCAGGATTATAACTCGTACA | |
| | | CGTTCGGAGGGGGGACCAAGCTGGAAATAAAACGG | |
| | | MKSQTQVFVFLLLCVSGAHGSIVMTQTPKFLLVSAGDRVTITCKAS | 44 |
| | | QSVSTDVAWYQQKPGQSPKLLIYYASNRYTGVPDRFTGSGYGTD | |
| | | FTFTISTVQAEDLAVYFCQQDYNSYTFGGGTKLEIKR | |
| | CDRL1 | AAGGCCAGTCAGAGTGTGAGTACTGATGTAGCT | 45 |
| | | KASQSVSTDVA | 46 |
| | CDRL2 | TATGCATCCAATCGCTACACT | 47 |
| | | YASNRYT | 48 |
| | CDRL3 | CAGCAGGATTATAACTCGTACACG | 49 |
| | | QQDYNSYT | 50 |

AB2074, AB2078

| | | | |
|---|---|---|---|
| VH | Full | ATGGAATGGAGCTGGATCTTTCTCTTCCTCCTGTCAGGAACTTCAG | 51 |
| | Length | GTGTCCACTCTGAGATCCAGCTGCAGCAGTCTGGACCTGAGCTG | |
| | (leader | GTGAACCCTGGGGCTTCAGTGAAGGTATCCTGCAAGGCTTCTG | |
| | sequence | GTTACTCATTCACTGACTACAACATGTACTGGGTGAAGCAG | |
| | in italics) | AGCCATGGAAAGAGCCTTGAGTGGATTGGATATATTGATCCT | |
| | | TACAATGGTGGTACTAGCTATAATCAGAAGTTCAAGGGCAA | |
| | | GGCCACATTGACTGTTGACAAGTCCTCCAGCACAGCCTTCATG | |
| | | CATCTCAACAGCCTGACATCTGAGGACTCTGCAGTCTATTACT | |
| | | GTGCAAGAGAGTACTACGGTAGTAGACTTTGGTACTTCGAT | |
| | | GTCTGGGGCGCAGGGACCGCGGTCACCGTCTCCTCA | |
| | | MEWSWIFLFLLSGTSGVHSEIQLQQSGPELVNPGASVKVSCKASGY | 52 |
| | | SFTDYNMYWVKQSHGKSLEWIGYIDPYNGGTSYNQKFKGKAT | |
| | | LTVDKSSSTAFMHLNSLTSEDSAVYYCAREYYGSRLWYFDVWG | |
| | | AGTAVTVSS | |
| | CDRH1 | GGTTACTCATTCACTGACTACAACATGTAC | 53 |
| | | GYSFTDYNMY | 54 |
| | CDRH2 | TATATTGATCCTTACAATGGTGGTACTAGCTATAATCAGAAGTT | 55 |
| | | CAAGGGC | |
| | | YIDPYNGGTSYNQKFKG | 56 |
| | CDRH3 | GAGTACTACGGTAGTAGACTTTGGTACTTCGATGTC | 57 |
| | | EYYGSRLWYFDV | 58 |
| VL | Full | ATGGAATCACAGACCCAGGTCCTCATGTTTCTTCTGCTCTGGGTAT | 59 |
| | Length | CTGGTGCCTGTGTAGACATTGTGATGACACAGTCTCCATCCTCCC | |
| | (leader | TGGCTATGTCAGTAGGACAGAAGGTCACTATGAGCTGCAAGTC | |
| | sequence | CAGTCAGAGTCTTTTAAATAGGAACAATCAAAAGAACTATT | |
| | in italics) | TGGCCTGGTACCAGCAGAAACCAGGACAGTCTCCTAAACTTCT | |
| | | GGTATACTTTGCATCCACTAGGGAATCTGGGGTCCCTGATCG | |
| | | CTTCATAGGCAGTGGATCTGGGACAGATTTCACTCTTACCCTCA | |
| | | GCAGTGTGCGGGCTGAAGACCTGGCAGAAAACTTCTGTCAGC | |
| | | AACATTATAGCACTCCGTTCACGTTCGGCTCGGGGACAAGGT | |
| | | TGGAAATAAAACGG | |
| | | MESQTQVLMFLLLWVSGACVDIVMTQSPSSLAMSVGQKVTMSCKS | 60 |
| | | SQSLLNRNNQKNYLAWYQQKPGQSPKLLVYFASTRESGVPDRFI | |
| | | GSGSGTDFTLTLSSVRAEDLAENFCQQHYSTPFTFGSGTRLEIKR | |
| | CDRL1 | AAGTCCAGTCAGAGTCTTTTAAATAGGAACAATCAAAAGAACT | 61 |
| | | ATTTGGCC | |
| | | KSSQSLLNRNNQKNYLA | 62 |
| | CDRL2 | TTTGCATCCACTAGGGAATCT | 63 |
| | | FASTRES | 64 |
| | CDRL3 | CAGCAACATTATAGCACTCCGTTCACG | 65 |
| | | QQHYSTPFT | 66 |

AB2079

| | | | |
|---|---|---|---|
| VH | Full | ATGAACTTCGGGCTCAGCTTGATTTTCCTTGTCCTTGTTTTAAAAGG | 67 |
| | Length | TGTCCAGTGTGAAGTGATGCTGGTGGAGTCTGGGGGAGGCTTA | |
| | (leader | GTGAAGCCTGGAGGGTCCCTGAAACTCTCCTGTGCAGCCTCTG | |
| | sequence | GATTCACTTTCAGTAACTATGCCATGTCTTGGGTTCGCCAGA | |
| | in Italics) | CTCCGGAGAAGAGGCTGGAGTGGGTCGCAAAAACTAGTAGTG | |
| | | GTGATACTTACACCTACTATCCAGACAGTGTGAAGGGGCGA | |
| | | TTCACCATCTCCAGGGACAATGCCAAGAACACCCTGTACCTGC | |
| | | AAATGACCAGTCTGAGGTCTGAGGACACGGCCATTTATTACTG | |
| | | TGCAAGACAAGACTACTACGGTATTACCCCTTGGTTCTTCG | |
| | | ATGTCTGGGGCGCAGGGACCACGGTCACCGTCTCCTCA | |
| | | MNFGLSLIFLVLVLKGVQCEVMLVESGGGLVKPGGSLKLSCAASG | 68 |
| | | FTFSNYAMSWVRQTPEKRLEWVAKTSSGDTYTYYPDSVKGRFT | |
| | | ISRDNAKNTLYLQMTSLRSEDTAIYYCARQDYYGITPWFFDVWG | |
| | | AGTTVTVSS | |
| | CDRH1 | GGATTCACTTTCAGTAACTATGCCATGTCT | 69 |
| | | GFTFSNYAMS | 70 |
| | CDRH2 | AAAACTAGTAGTGGTGATACTTACACCTACTATCCAGACAGTG | 71 |
| | | TGAAGGGG | |
| | | KTSSGDTYTYYPDSVKG | 72 |
| | CDRH3 | CAAGACTACTACGGTATTACCCCTTGGTTCTTCGATGTC | 73 |
| | | QDYYGITPWFFDV | 74 |
| VL | Full | ATGGATTTTCTGGTGCAGATTTTCAGCTTCTTGCTAATCAGTGCCTC | 75 |
| | Length | AGTTGCAATGTCCAGAGGAGAAAATGTGCTCACCCAGTCTCCAG | |
| | (leader | CAATCATGTCTGCATCTCCAGGGGAAAAGGTCACCATGACCTG | |
| | sequence | CAGGGCCAGCTCAAGTGTAAGTTCCATTTACTTGCACTGGT | |
| | in italics) | ACCAGCAGAAGTCAGGTGCCTCCCCCAAACTCTGGATTTATAG | |
| | | CACATCCAACTTGGCTTCTGGAGTCCCTGCTCGCTTCAGTGG | |
| | | CAGTGGGTCTGGGACCTCTTACTCTCTCACAATCACCAGTGTG | |
| | | GAGGCTGAAGATGCTGCCACTTATTACTGCCAGCAGTACAGT | |
| | | GGTTACCCACTCATCACGTTCGGTGCTGGGACCAAGCTGGAG | |
| | | CTGAAACGG | |
| | | MDFLVQIFSFLLISASVAMSRGENVLTQSPAIMSASPGEKVTMTCRA | 76 |
| | | SSSVSSIYLHWYQQKSGASPKLWIYSTSNLASGVPARFSGSGSGT | |
| | | SYSLTITSVEAEDAATYYCQQYSGYPLITFGAGTKLELKR | |
| | CDRL1 | AGGGCCAGCTCAAGTGTAAGTTCCATTTACTTGCAC | 77 |
| | | RASSSVSSIYLH | 78 |
| | CDRL2 | AGCACATCCAACTTGGCTTCT | 79 |
| | | STSNLAS | 80 |
| | CDRL3 | CAGCAGTACAGTGGTTACCCACTCATCACG | 81 |
| | | QQYSGYPLIT | 82 |

AB2092

| | | | |
|---|---|---|---|
| VH | Full | ATGGATTGGCTGTGGAACTTGCTATTCCTGATGGCAGCTGCCCAAA | 83 |
| | Length | GTGCCCAAGCACAGATCCAGTTGGTGCAATCTGGACCTGAGCT | |
| | (leader | GAAGAAGTCTGGAGAGACAGTCAAGATCTCCTGCAAGGCTTC | |
| | sequence | TGGGTATACCTTCACAGACTATGGAATGAACTGGGTGAAGC | |
| | in Italics) | AGGCTCCAGGAAAGGGTTTAAAGTGGATGGGCTGGATCAACA | |
| | | CCTACACTGGAAAGTCAACATATGCTGATGACTTCAAGGGA | |
| | | CGGTTTGCCTTCTCTTTGGAAACCTCTGCCAGCACTACCTATTT | |
| | | GCAGATCACCAACCTCAAAAATGAGGACATGGCTACATATTTC | |
| | | TGTGCGAGATGGGCGGATTACTTCGGTAGTAGCTACCTGTT | |
| | | TGCTTCCTGGGGCCAAGGGACTCTGGTCACTGTCTCTGCA | |
| | | MDWLWNLLFLMAAAQSAQAQIQLVQSGPELKKSGETVKISCKASG | 84 |
| | | YTFTDYGMNWVKQAPGKGLKWMGWINTYTGKSTYADDFKGR | |
| | | FAFSLETSASTTYLQITNLKNEDMATYFCARWADYFGSSYLFAS | |
| | | WGQGTLVTVSA | |
| | CDRH1 | AAGGCTTCTGGGTATACCTTCACAGACTATGGAATGAAC | 85 |
| | | KASGYTFTDYGMN | 86 |
| | CDRH2 | TGGATCAACACCTACACTGGAAAGTCAACATATGCTGATGACT | 87 |
| | | TCAAGGGA | |
| | | WINTYTGKSTYADDFKG | 88 |
| | CDRH3 | TGGGCGGATTACTTCGGTAGTAGCTACCTGTTTGCTTCC | 89 |
| | | WADYFGSSYLFAS | 90 |
| VL | Full | ATGAAGTTGCCTGTTAGGCTGTTGGTGCTGATGTTCTGGATTCCTG | 91 |
| | Length | TTTCCAGCAGTGATGTTTTGATGACCCAAACTCCACTCTCCCTGC | |
| | (leader | CTGTCAGTCTTGGAGATCAAGCCTCCATCTCTTGCAGATCTAG | |
| | sequence | TCAGAGCATTGTACATAGTGATGGAAACACCTATTTAGAAT | |
| | in italics) | GGTACCTGCAGAAACCAGGCCAGTCTCCAAAGCTCCTGATCTA | |
| | | CAAAGTTTCCAACCGATTTTCTGGGGTCCCAGACAGGTTCAG | |
| | | TGGCAGTGGATCAGGGACAGATTTCACACTCAAGATCAGCAGA | |
| | | GTGGAGGCTGAGGATCTGGGAATTTATTACTGCTTTCAAGGTT | |
| | | CACATGTTCCGTGGACGTTCGGTGGAGGCACCAAGCTGGAAA | |
| | | TCAAACGG | |
| | | MKLPVRLLVLMFWIPVSSSDVLMTQTPLSLPVSLGDQASISCRSSQS | 92 |
| | | IVHSDGNTYLEWYLQKPGQSPKWYKVSNRFSGVPDRFSGSGSG | |
| | | TDFTLKISRVEAEDLGIYYCFQGSHVPWTFGGGTKLEIKR | |
| | CDRL1 | AGATCTAGTCAGAGCATTGTACATAGTGATGGAAACACCTATT | 93 |
| | | TAGAA | |
| | | RSSQSIVHSDGNTYLE | 94 |
| | CDRL2 | AAAGTTTCCAACCGATTTTCT | 95 |
| | | KVSNRFS | 96 |
| | CDRL3 | TTTCAAGGTTCACATGTTCCGTGGACG | 97 |
| | | FQGSHVPWT | 98 |

### Example 2

### Characterization of DDR1Antibodies

The ten antibodies were assessed for binding to DDR1, with the results presented in Table 2 below. Antibodies were incubated with an HEK293 cell line engineered to stably express human DDR1 (isoform B). Flow cytometry (FACS) was used to assess binding of the antibodies to this cell line, measured as Mean Fluorescent Intensity (MFI). An HEK293 cell line stably transfected with an empty vector was also stained with the same antibody. The MFI values listed in Table 1 below are normalized to the MFI for this HEK293/empty vector control.

K_{d} values, measured via ELISA, also are listed in Table 2. No binding was detected by any of the antibodies to a DDR2ECD-Fc fusion protein.

TABLE 2
Anti-DDR1 Monoclonal Antibody Antigen Binding

| | Binding to | | |
|---|---|---|---|
| | HEK293/humanDDR1 | DDR1 ECD-Fc | DDR2 ECD-Fc |
| Antibody | (MFI)¹ | K_{d} (nM)² | K_{d} (nM) |
| AB2004 | 14434 | 0.154 | No Binding |
| AB2009 | 11920 | 0.257 | No Binding |
| AB2026 | 6352 | 0.298 | No binding |
| AB2039 | 8019 | 0.346 | No binding |
| AB2031 | 8540 | 0.257 | No Binding |
| AB2041 | 12484 | 0.561 | No Binding |
| AB2074 | 7806 | 0.259 | No Binding |
| AB2078 | 7491 | 0.374 | No Binding |
| AB2079 | 14280 | 0.088 | No Binding |
| AB2092 | 6018 | 0.287 | No Binding |

### Example 3

### Additional Anti-DDR1Antibodies

Additional monoclonal antibodies were obtained from individual hybridomas in Example 1 and were characterized for their properties. The binding of anti-DDR1 monoclonal antibodies to DDR1 and DDR2 proteins were examined in human epithelial kidney (HEK) 293 cells that were stably transfected with either human DDR1, human DDR2, mouse DDR1, or empty vector, as well as two human cancer cell lines exhibiting endogenous expression of DDR1 receptor: HCT-116 colorectal carcinoma and T47D breast cancer.

Cells were cultured in DMEM with 10% FBS and 0.8 mg/mL hygromycin. When cells reached 60-80% confluence, they were centrifuged at 1,200 rpm for 5 minutes, suspended in FACS buffer (1×PBS, 1% BSA), and aliquoted into a 96-well plate at 1×10⁶ cells per well. The anti-DDR1 monoclonal antibodies or appropriate isotype controls at 2 μg per 1×10⁶ cells were added and incubated at 4° C. for 1 hr. The cells were washed twice with FACS buffer then stained with the anti-IgG-PE secondary antibody at 10 μL per well at 4° C. for 1 hr. The cells were washed again and measured for the MFI values or signals using flow cytometry (LSR Fortessa instrument, BD Biosciences). Background MFI signals from vector-only cells (i.e. no DDR1 or DDR2 expression) was subtracted from those of the cells treated with antibodies. Higher MFI values or signals indicated increased binding or specificity of the anti-DDR1 antibodies to the DDR1 on the cell surface.

Table 3 summarized the MFI values of the cells treated with certain anti-DDR1 monoclonal antibodies. Some results were provided in Table 2. As shown in Tables 2 and 3, all of the tested anti-DDR1 monoclonal antibodies had more than 20 fold increased binding or specificity to human DDR1 compared to human DDR2. For example, the MFI value of AB2039 in the cells expressing human DDR1 was about 56 fold increased compared to those in the cells expressing human DDR2. Also, the MFI value of AB2041 in the cells expressing human DDR1 was about 47 fold increased compared to those in the cells expressing human DDR2. These results indicate that the anti-DDR1 monoclonal antibodies provided herein exhibit the binding or specificity to DDR1. Moreover, certain antibodies, including AB2004, AB2012, AB2039, AB2041, AB2078, and AB2092, exhibit the binding or specificity to both human and murine DDR1 proteins.

Table 3. The MFI values of anti-DDR1 antibodies.

TABLE 3
The MFI Values of Anti-DDR1 Antibodies

| | MFI | | | | |
|---|---|---|---|---|---|
| | FACS | MFI FACS | MFI FACS | MFI FACS | |
| Antibody | hDDR1 | hDDR2 | T47D | HCT116 | |
| AB2002 | 7243 | 170 | 3594 | 1012 | N/A |
| AB2003 | 15027 | 176 | 6098 | 1407 | NA |
| AB2004 | 14434 | 289 | 5990 | 1488 | 9505 |
| AB2009 | 11920 | 277 | 5930 | 1481 | N/A |
| AB2010 | 17805 | 647 | 6728 | 1726 | 7138 |
| AB2012 | 17270 | 448 | 7410 | 1870 | 52000 |
| AB2019 | 12578 | 244 | 4949 | 1490 | N/A |
| AB2021 | 15758 | 92 | 5007 | 1389 | N/A |
| AB2025 | 61 | 3 | 57 | 36 | N/A |
| AB2026 | 6352 | 203 | 4668 | 1539 | N/A |
| AB2029 | 11685 | 263 | 3557 | 1013 | N/A |
| AB2031 | 8540 | 195 | 4599 | 1459 | N/A |
| AB2032 | 13673 | 265 | 6824 | 1897 | N/A |
| AB2034 | 11449 | 175 | 4370 | 1058 | N/A |
| AB2039 | 8019 | 143 | 4464 | 1486 | 10500 |
| AB2040 | 13675 | 308 | 2813 | 2214 | N/A |
| AB2041 | 12484 | 265 | 2206 | 1274 | N/A |
| AB2049 | 11655 | 322 | 5025 | 1511 | N/A |
| AB2054 | 15511 | 306 | 5932 | 1528 | N/A |
| AB2061 | 7497 | 384 | 4617 | 1197 | N/A |
| AB2063 | 1438 | 0 | 615 | 111 | N/A |
| AB2065 | 3563 | 353 | 4404 | 2479 | N/A |
| AB2073 | 16216 | 410 | 6052 | 1691 | N/A |
| AB2074 | 7806 | 382 | 4420 | 1472 | N/A |
| AB2078 | 7491 | 380 | 4751 | 1484 | 24200 |
| AB2079 | 14280 | 326 | 6129 | 1728 | N/A |
| AB2080 | 3140 | 313 | 1215 | 572 | N/A |
| AB2085 | 8837 | 126 | 3766 | 943 | N/A |
| AB2092 | 6018 | 133 | 3880 | 1313 | 22800 |
| AB2154 | 15434 | 305 | N/A | N/A | N/A |
| AB2163 | 19949 | 304 | N/A | N/A | N/A |

N/A: not available

Table 4 summarized the sequences of the antibodies designated as AB2002, AB2010, AB2019, AB2021, AB2029, AB2032, AB2034, AB2049, AB2080, and AB2085. The nucleotide and amino acid sequences for the variable heavy (VH) chain and variable light (VL) chain for each antibody were provided, with the leader sequence set forth in italics and complementarity determining region (CDR) sequences set forth in bold. Individual CDR1, CDR2, and CDR3 sequences also were also provided for each VH and VL sequence. The sequence analysis indicated that AB2010, AB2019 and AB2032 were identical; AB2009, AB2026, AB2031 and AB2039 were identical; AB2003 and AB2004 were identical; AB2040 AB2054, AB2065, AB2073 and AB2079 were identical; and AB2061, AB2074 and AB2078 were identical.

TABLE 4
Additional Anti-DDR1 Monoclonal Antibodies

| | |
|---|---|
| | SEQ |
| | ID |
| Sequences | NO |

AB2010, AB2019, AB2032

| | | | |
|---|---|---|---|
| VH | Full | ATGGCTGTCTTGGGGCTGCTCTTCTGCCTGGTGACATTCCCAAGCT | 99 |
| | Length | GTGTCCTATCCCAGGTGCAGCTGAAGCAGTCAGGACCTGGCCT | |
| | (leader | AGTGCAGCCCTCACAGAGCCTGTCCATCACCTGCACAGTCTCT | |
| | sequence | GGTTTCTCATTTACTGGCTATGGTATACACTGGGTTCGCCAG | |
| | in italics) | TCTCCAGGAAAGGGTCTGGAGTGGCTCGGAGTGATTTGGAGT | |
| | | GGTGGAATCACAGACTATAATGCAGCTTTCATATCCAGACT | |
| | | GAGCATCAGCATGGACAGTTCCAAGGGCCAAGTTTTCTTTAAA | |
| | | ATGAACAGTCTGCAAGCTAATGACACAGCCATATATTACTGTG | |
| | | CCAGAAGAAGTACTACGGTAGTGGCCGACTATGCTATGGAC | |
| | | TACTGGGGTCAAGGAACCTCAGTCACCGTCTCCTCA | |
| | | MAVLGLLFCLVTFPSCVLSQVQLKQSGPGLVQPSQSLSITCTVSGFS | 100 |
| | | FTGYGIHWVRQSPGKGLEWLGVIWSGGITDYNAAFISRLSISMD | |
| | | SSKGQVFFKMNSLQANDTAIYYCARRSTTVVADYAMDYWGQG | |
| | | TSVTVSS | |
| | CDRH1 | GGTTTCTCATTTACTGGCTATGGTATACAC | 101 |
| | | GFSFTGYGIH | 102 |
| | CDRH2 | GTGATTTGGAGTGGTGGAATCACAGACTATAATGCAGCTTTCA | 103 |
| | | TATCC | |
| | | VIWSGGITDYNAAFIS | 104 |
| | CDRH3 | AGAAGTACTACGGTAGTGGCCGACTATGCTATGGACTAC | 105 |
| | | RSTTVVADYAMDY | 106 |
| VL | Full | ATGAAGTCACAGACCCAGGTCTTCGTATTTcTACTGCTCTGTG | 107 |
| | Length | TGTCTGGTGCTCATGGGAGTATTGTGATGACCCAGACTCC | |
| | (leader | CAAGTTCCTGCTTGTATCAGCAGGAGACAGGGTTACCAT | |
| | sequence | AACCTGCAAGGCCAGTCAGAGTGTGAGTAATGATGTA | |
| | in italics) | GCTTGGTACCAACAGAAgCCAGGGCAGTCTCCTAAACTG | |
| | | CTGATATACTATGCATCCAATCGCTACACTGGAGTCCCT | |
| | | GATCGCTTCACTGGCAGTGGATATGGGACGGATTTCACTT | |
| | | TCACCATCAACACTGTGCAGGCTGAAGACCTGGCAGTTT | |
| | | ATTTCTGTCAACAGGATTATACCTCTCCTCTCACGTTCG | |
| | | GTGCTGGGACCAAGCTGGAGCTGAAACGG | |
| | | MKSQTQVFVFLLLCVSGAHGSIVMTQTPKFLLVSAGDRVTITC | 108 |
| | | KASQSVSNDVAWYQQKPGQSPKLLIYYASNRYTGVPDRFT | |
| | | GSGYGTDFTFTINTVQAEDLAVYFCQQDYTSPLTFGAGTKL | |
| | | ELKR | |
| | CDRL1 | AAGGCCAGTCAGAGTGTGAGTAATGATGTAGCT | 109 |
| | | KASQSVSNDVA | 110 |
| | CDRL2 | TATGCATCCAATCGCTACACT | 111 |
| | | YASNRYT | 112 |
| | CDRL3 | CAACAGGATTATACCTCTCCTCTCACG | 113 |
| | | QQDYTSPLT | 114 |

AB2029

| | | | |
|---|---|---|---|
| VH | Full | ATGGCTGTCTTGGGGCTGCTCTTCTGCCTGGTGACATTCCCA | 115 |
| | Length | AGCTGTGTCCTATCCCAGGTGCTGCTGAGGCAGTCAGGAC | |
| | (leader | CTGGCCTAGTGCAGCCCTCACAGAGCCTGTCCATCACCTG | |
| | sequence | CACAGTCTCTGGTTTCTCATTAACTACCTATGGTGTAC | |
| | in italics) | ACTGGGTTCGCCAGTCTCCAGGAAAGGGTCTGGAGTGGC | |
| | | TGGGAGTGATATGGAGTGGTGGAAGCACAGACTATAA | |
| | | TGCAGCTTTCATATCCAGACTGACCATCAGCAAGGACA | |
| | | ATTCCAAGAGCCAAGTTTTCTTTAAAATGAACAGTCTGCA | |
| | | AGCTAATGACACAGCCATATATTACTGTGCCAGATATTA | |
| | | CTCCGGTAGTAGCCTTTACTTTGCTATGGACTACTGGG | |
| | | GTCAAGGAACCTCAGTCACCGTCTCCTCA | |
| | | MAVLGLLFCLVTFPSCVLSQVLLRQSGPGLVQPSQSLSITCTV | 116 |
| | | SGFSLTTYGVHWVRQSPGKGLEWLGVIWSGGSTDYNAAF | |
| | | ISRLTISKDNSKSQVFFKMNSLQANDTAIYYCARYYSGSSLY | |
| | | FAMDYWGQGTSVTVSS | |
| | CDRH1 | GGTTTCTCATTAACTACCTATGGTGTACAC | 117 |
| | | GFSLTTYGVH | 118 |
| | CDRH2 | GTGATATGGAGTGGTGGAAGCACAGACTATAATGCAGCT | 119 |
| | | TTCATATCC | |
| | | VIWSGGSTDYNAAFIS | 120 |
| | CDRH3 | TATTACTCCGGTAGTAGCCTTTACTTTGCTATGGACTAC | 121 |
| | | YYSGSSLYFAMDY | 122 |
| VL | Full | ATGAAGTCACAGACCCAGGTCTTCGTATTTCTACTGCTCTGTG | 123 |
| | Length | TGTCTGGTGCTCATGGGAATATTGTGATGACCCAGACTCC | |
| | (leader | CAAATTCCTGCTTGTATCAGCAGGAGACACGGTTACCATA | |
| | sequence | ACCTGCAAGGCCAGTCAGAGTGTGAGTAATGATGTTG | |
| | in italics) | CTTGGTACCAACAGAAGCCAGGGCAGTCTCCTAAACTGC | |
| | | TGATATACTATGCATCCAATCGCTACACTGGAGTCCCTG | |
| | | ATCGCTTCACTGGCAGTGGATTTGGGACGGATTTCACTTT | |
| | | CACCATCAGCACTGTGCAGACTGAAGACCTGGCAGTTTA | |
| | | TTTCTGTCAGCAGGATTATAGCTCGCTCACGTTCGGTGC | |
| | | TGGGACCAAGCTGGAGCTGAAACGG | |
| | | MKSQTQVFVFLLLCVSGAHGNIVMTQTPKFLLVSAGDTVTI | 124 |
| | | TCKASQSVSNDVAWYQQKPGQSPKLLIYYASNRYTGVPD | |
| | | RFTGSGFGTDFTFTISTVQTEDLAVYFCQQDYSSLTFGAGTK | |
| | | LELKR | |
| | CDRL1 | AAGGCCAGTCAGAGTGTGAGTAATGATGTTGCT | 125 |
| | | KASQSVSNDVA | 126 |
| | CDRL2 | TATGCATCCAATCGCTACACT | 127 |
| | | YASNRYT | 128 |
| | CDRL3 | CAGCAGGATTATAGCTCGCTCACG | 129 |
| | | QQDYSSLT | 130 |

AB2034

| | | | |
|---|---|---|---|
| VH | Full | ATGGCTGTCCTGGGGCTGCTTCTCTGCCTGGTGACTTTCCCA | 131 |
| | Length | AGCTGTGTCCTGTCCCAGGTACAGTTGAAGGAGTCAGGAC | |
| | (leader | CTGGCCTGGTGGCGCCCTCACAGAGCCTGTCCATCACATG | |
| | sequence | CACCGTCTCAGGGTTTTCATTAACTAGTTATCATATAC | |
| | in italics) | ACTGGGTTCGTCAGCCTCCAGGAAAGGGTCTGGAGTGGC | |
| | | TGGTAGTGATATGGAGTGATGGAAGCACAACCTATAA | |
| | | TTCAGCTCTCAAATCCAGACTGAGCATCAGCAAGGACA | |
| | | ACTCCAAGAGCCAAGTTTTCTTAAAAATGAACAGTCTCCA | |
| | | AACTGATGACACAGCCATTTACTACTGTGCCAGAACTGG | |
| | | TAATTTTTATGCTATGGACTACTGGGGTCAAGGAACCT | |
| | | CAGTCACCGTCTCCTCA | |
| | | MAVLGLLLCLVTFPSCVLSQVQLKESGPGLVAPSQSLSITCTV | 132 |
| | | SGFSLTSYHIHWVRQPPGKGLEWLVVIWSDGSTTYNSALK | |
| | | SRLSISKDNSKSQVFLKMNSLQTDDTAIYYCARTGNFYAMD | |
| | | YWGQGTSVTVSS | |
| | CDRH1 | GGGTTTTCATTAACTAGTTATCATATACAC | 133 |
| | | GFSLTSYHIH | 134 |
| | CDRH2 | GTGATATGGAGTGATGGAAGCACAACCTATAATTCAGCT | 135 |
| | | CTCAAATCC | |
| | CDRH3 | VIWSDGSTTYNSALKS | 136 |
| | | ACTGGTAATTTTTATGCTATGGACTAC | 137 |
| | | TGNFYAMDY | 138 |
| VL | Full | ATGAAGTCACAGACCCAGGTCTTCGTATTTCTACTGCTCTGTG | 139 |
| | Length | TGTCTGGTGCTCATGGGAGTATTGTGATGACCCAGACTCC | |
| | (leader | CAAATTCCTGCTTGTATCAGCAGGAGACAGGGTTACCAT | |
| | sequence | AACCTGCAAGGCCAGTCAGAGTGTGAGTAATGATGTA | |
| | in italics) | GTTTGGTACCAACAGAAGCCAGGGCAGTCTCCTAAACTG | |
| | | CTGATATAYTATGCATCCAGTCGCTACACTGGAGTCCCT | |
| | | GATCGCTTCACTGGCAGTGGATATGGGACGGATTTCACTT | |
| | | TCACCATCAGCACTGTGCAGGCTGAAGACCTGGCAGTTT | |
| | | ATTTCTGTCAGCAGGATTATAGCTCTCCGTACACGTTC | |
| | | GGAGGGGGGACCAAGCTGGAAATAAGA | |
| | | MKSQTQVFVFLLLCVSGAHGSIVMTQTPKFLLVSAGDRVTITC | 140 |
| | | KASQSVSNDVVWYQQKPGQSPKLLIYYASSRYTGVPDRFT | |
| | | GSGYGTDFTFTISTVQAEDLAVYFCQQDYSSPYTFGGGTKL | |
| | | EIR | |
| | CDRL1 | AAGGCCAGTCAGAGTGTGAGTAATGATGTAGTT | 141 |
| | | KASQSVSNDVV | 142 |
| | CDRL2 | TATGCATCCAGTCGCTACACT | 143 |
| | | YASSRYT | 144 |
| | CDRL3 | CAGCAGGATTATAGCTCTCCGTACACG | 145 |
| | | QQDYSSPYT | 146 |

AB2002

| | | | |
|---|---|---|---|
| VH | Full | ATGAACTTCGGGTTCAGCTTGATTTTCCTTGTCCTTGTTTTAAA | 147 |
| | Length | AGGTGTCCAGTGTGAAGTGAACCTGGTGGAGTCTGGGGGA | |
| | (leader | GGCTTAGTGAAGCCTGGAGGGTCCCTGAAACTCTCCTGTG | |
| | sequence | CAGCCTCTGGATTCACTTTCAGTACCTATGCCATGTCT | |
| | in italics) | TGGGTTCGCCAAACTCCAGAGAAGAGGCTGGAGTGGGTC | |
| | | GCATCCATTAGTAGTGGTGGTATCACCCACTATCCAG | |
| | | ACAGTATGAAGGGCCGATTCACCGTCTCCAGAGATAAT | |
| | | GCCAGGAACATCCTGTACCTGCAAATGCGCAGTCTGAGG | |
| | | TCTGAGGACACGGCCAAGTATTACTGTGCAAGGGGTCGA | |
| | | TCTTCTATGATCCCCGACTACTGGGGTCAAGGAACCTC | |
| | | AGTCTCCGTCTCCTCA | |
| | | MNFGFSLIFLVLVLKGVQCEVNLVESGGGLVKPGGSLKLSCA | 148 |
| | | ASGFTFSTYAMSWVRQTPEKRLEWVASISSGGITHYPDSM | |
| | | KGRFTVSRDNARNILYLQMRSLRSEDTAKYYCARGRSSMI | |
| | | PDYWGQGTSVSVSS | |
| | CDRH1 | GGATTCACTTTCAGTACCTATGCCATGTCT | 149 |
| | | GFTFSTYAMS | 150 |
| | CDRH2 | TCCATTAGTAGTGGTGGTATCACCCACTATCCAGACAGTA | 151 |
| | | TGAAGGGC | |
| | | SISSGGITHYPDSMKG | 152 |
| | CDRH3 | GGTCGATCTTCTATGATCCCCGACTAC | 153 |
| | | GRSSMIPDY | 154 |
| VL | Full | ATGGAGACACATTCTCAGGTCTTTGTATACATGTTGCTGTGGT | 211 |
| | Length | TGTCTGGTGTTGAAGGAGACATTGTGATGACCCAGTCTCAC | |
| | (leader | AAATTCATGTCCACATCAGTAGGAGACAGGGTCAGCATC | |
| | sequence | ACCTGCAAGGCCAGTCAGGATGTGGGTACTGCTGTAG | |
| | in italics) | CCTGGTATCAACAGAAACCAGGACAATCTCCTAAACTAC | |
| | | TGATTTACTGGGCATCCACCCGGCACACTGGAGTCCCT | |
| | | GATCGCTTCACAGGCAGTGGATCTGGGACAGATTTCACTC | |
| | | TCACCATTAGCAATGTGCAGTCTGAAGACTTGGCAGATTA | |
| | | TTTCTGTCAGCAATACAGCACCTATCCTCTCACGTTCGG | |
| | | TGCTGGGACCAAGCTGGAGCTGAAACGG | |
| | | METHSQVFVYMLLWLSGVEGDIVMTQSHKFMSTSVGDRVSIT | 212 |
| | | CKASQDVGTAVAWYQQKPGQSPKLLIYWASTRHTGVPDR | |
| | | FTGSGSGTDFTLTISNVQSEDLADYFCQQYSTYPLTFGAGT | |
| | | KLELKR | |
| | CDRL1 | AAGGCCAGTCAGGATGTGGGTACTGCTGTAGCC | 213 |
| | | KASQDVGTAVA | 214 |
| | CDRL2 | TGGGCATCCACCCGGCACACT | 215 |
| | | WASTRHT | 216 |
| | CDRL3 | CAGCAATACAGCACCTATCCTCTCACG | 217 |
| | | QQYSTYPLT | 218 |

AB2021

| | | | |
|---|---|---|---|
| VH | Full | ATGAACTTCGGGTTCAGCTTGATTTTCCTTGTCCTTGTTTTAAA | 155 |
| | Length | AGGTGTCCAGTGTGAAGTGAAGCTGGTGGAGTCTGGGGGA | |
| | (leader | GGCTTAGTGAAGCCTGGAGGGTCCCTGAACCTCTCCTGTG | |
| | sequence | CAGCCTCTGGATTCACTTTCAGTAACTATGCCATGTCT | |
| | in italics) | TGGTCTCGCCAGACTCCAGAGAAGAGGCTGGAGTGGGTC | |
| | | GCATCCATTAGTAGTGGTGGTAACACCTACTATCCAG | |
| | | ACAGTGTGAGGGGCCGATTCACCATCTCCAGAGATAAT | |
| | | GCCAGGAACATCCTGTACCTGCAAATGAGCAGTCTGAGG | |
| | | TCTGAGGACACGGCCATGTATTACTGTGCAAGAGAGCCC | |
| | | GGTACCTACAACTGGTACTTCGATGTCTGGGGCGCAGG | |
| | | GACCACGGTCACCGTCTCCTCA | |
| | | MNFGFSLIFLVLVLKGVQCEVKLVESGGGLVKPGGSLNLSCA | 156 |
| | | ASGFTFSNYAMSWSRQTPEKRLEWVASISSGGNTYYPDSV | |
| | | RGRFTISRDNARNILYLQMSSLRSEDTAMYYCAREPGTYN | |
| | | WYFDVWGAGTTVTVSS | |
| | CDRH1 | GGATTCACTTTCAGTAACTATGCCATGTCT | 157 |
| | | GFTFSNYAMS | 158 |
| | CDRH2 | TCCATTAGTAGTGGTGGTAACACCTACTATCCAGACAGTG | 159 |
| | | TGAGGGGC | |
| | | SISSGGNTYYPDSVRG | 160 |
| | CDRH3 | GAGCCCGGTACCTACAACTGGTACTTCGATGTC | 161 |
| | | EPGTYNWYFDV | 162 |
| VL | Full | ATGGAGTCACAGACTCAGGTCTTTGTATACATGTTGCTGTGGT | 219 |
| | Length | TGTCTGGTGTTGATGGAGACATTGTGATGACCCAGTCTCAA | |
| | (leader | AAATTCATGTCCACATCAGTAGGAGACAGGGTCAGCGTC | |
| | sequence | ACCTGCAAGGCCAGTCAGAATGTGGGTACTAATGTAG | |
| | in italics) | CCTGGTATCAACAGAAACCAGGGCAATCTCTTAAAGCAC | |
| | | TGATTTACTCGGCATCCTACCGGTTCAGTGGAGTCCCTG | |
| | | ATCGCTTCACAGGCAGTGGATATGGGACAGATTTCACTCT | |
| | | CACCATCAGCAATGTGCAGTCTGAAGACTTGGCAGAGTA | |
| | | TTTCTGTCAGCAATGTAACAGCTATCCTCTCACGTTCGG | |
| | | TGCTGGGACCAAGCTGGAGCTGAAACGG | |
| | | MESQTQVFVYMLLWLSGVDGDIVMTQSQKFMSTSVGDRVSV | 220 |
| | | TCKASQNVGTNVAWYQQKPGQSLKALIYSASYRFSGVPDR | |
| | | FTGSGYGTDFTLTISNVQSEDLAEYFCQQCNSYPLTFGAGT | |
| | | KLELKR | |
| | CDRL1 | AAGGCCAGTCAGAATGTGGGTACTAATGTAGCC | 221 |
| | | KASQNVGTNVA | 222 |
| | CDRL2 | TCGGCATCCTACCGGTTCAGT | 223 |
| | | SASYRFS | 224 |
| | CDRL3 | CAGCAATGTAACAGCTATCCTCTCACG | 225 |
| | | QQCNSYPLT | 226 |

AB2049

| | | | |
|---|---|---|---|
| VH | Full | ATGGCTGTCTTGGGGCTGCTCTTCTGCCTGGTGACATTCCCA | 163 |
| | Length | AGCTGTGTCCTATCCCAGGTGCAGCTGAAGCAGTCAGGAC | |
| | (leader | CTGGCCTAGTGCAGCCCTCACAGAGCCTGTCCATCACCTG | |
| | sequence | CACAGTCTCTGGTTTCTCATTAACTACCTATGGTGTAC | |
| | in italics) | ACTGGTTTCGCCAGTCTCCAGGAAAGGGTCTGGAGTGGC | |
| | | TGGGAGTGATATGGAGTGGTGGAAGCACAGACTTTAA | |
| | | TGCAGCTTTCATATCCAGACTGACCATCAACAAGGACA | |
| | | ATTCCAAGAGCCAAGTTTTCTTTACAATGAACAGTCTGCA | |
| | | AGCTAATGACACCGCCATATATTACTGTGCCAGAATTAG | |
| | | TACGGTACTAGCCAATTACTATCCTTTGGACTACTGGG | |
| | | GTCAAGGAACCTCAGTCACCGTCTCCTCA | |
| | | MAVLGLLFCLVTFPSCVLSQVQLKQSGPGLVQPSQSLSITCTV | 164 |
| | | SGFSLTTYGVHWFRQSPGKGLEWLGVIWSGGSTDFNAAFI | |
| | | SRLTINKDNSKSQVFFTMNSLQANDTAIYYCARISTVLANY | |
| | | YPLDYWGQGTSVTVSS | |
| | CDRH1 | GGTTTCTCATTAACTACCTATGGTGTACAC | 165 |
| | | GFSLTTYGVH | 166 |
| | CDRH2 | GTGATATGGAGTGGTGGAAGCACAGACTTTAATGCAGCT | 167 |
| | | TTCATATCC | |
| | | VIWSGGSTDFNAAFIS | 168 |
| | CDRH3 | ATTAGTACGGTACTAGCCAATTACTATCCTTTGGACTAC | 169 |
| | | ISTVLANYYPLDY | 170 |
| | CDRL1 | AAGACCAGTCAGAGTGTGAGTAATGATGTAGCT | 171 |
| | | KTSQSVSNDVA | 172 |
| | CDRL2 | TATGCATCCAATCGCTACACT | 173 |
| | | YASNRYT | 174 |
| | CDRL3 | CACCAGGATTATAGGTCTCCGCTCACG | 175 |
| | | HQDYRSPLT | 176 |

AB2080

| | | | |
|---|---|---|---|
| VH | Full | GAGGTCCAGCTGCAACAGTCTGGACCTGAGCTGGTGAAG | 177 |
| | Length | CCTGGAGCTTCAATGAAGATATCCTGCAAGGCTTCTGGT | |
| | without | TACTCATTCACTGGCTACACCATGACCTGGGTGAAGCA | |
| | full | GAGCCATGGAAAGAACCTTGAGTGGATTGGACTTATTAA | |
| | signal | TCCTTACATTGGTGTTACTAGCTACAACCAGAAGTTCA | |
| | peptide | AGGGCAAGGCCACATTGACTGTAGACAAGTCATCCAGCA | |
| | | CAGCCTACATGGAGCTCCTCAGTCTGACATCTGAGGACTC | |
| | | TGCAGTCTATTACTGTGCAAGACTCTATGATCGAGGTGG | |
| | | ACGCTATGCTATGGACTACTGGGGTCAAGGAACCTCAG | |
| | | TCACCGTCTCCTCA | |
| | | EVQLQQSGPELVKPGASMKISCKASGYSFTGYTMTWVKQS | 178 |
| | | HGKNLEWIGLINPYIGVTSYNQKFKGKATLTVDKSSSTAY | |
| | | MELLSLTSEDSAVYYCARLYDRGGRYAMDYWGQGTSVTV | |
| | | SS | |
| | CDRH1 | GGTTACTCATTCACTGGCTACACCATGACC | 179 |
| | | GYSFTGYTMT | 180 |
| | CDRH2 | CTTATTAATCCTTACATTGGTGTTACTAGCTACAACCAGA | 181 |
| | | AGTTCAAGGGC | |
| | | LINPYIGVTSYNQKFKG | 182 |
| | CDRH3 | CTCTATGATCGAGGTGGACGCTATGCTATGGACTAC | 183 |
| | | LYDRGGRYAMDY | 184 |
| VL | Full | CAAATTGTTCTCACCCAGTCTCCAGCAATCATGTCTGCAT | 185 |
| | Length | CTCCAGGGGAGAAGGTCCCCATGACCTGCAGTGCCAGCT | |
| | without | CAAGTGTAAGTTTCATGCACTGGTACCAGCAGAAGTCA | |
| | full | GGCACCTCCCCCAAAAGATGGATTTATGACACATCCAAA | |
| | signal | CTGGCTTCTGGAGTCCCTGCTCGCTTCAGTGGCAGTGGG | |
| | peptide | TCTGGGACCTCTTACTCTCTCACAATCAGCAACATGGAGG | |
| | | CTGAAGATGCTGCCACTTATTACTGCCAGCAGTGGAATA | |
| | | GTGACCCGTGGACGTTCGGTGGAGGCTCCAAGCTT | |
| | | QIVLTQSPAIMSASPGEKVPMTCSASSSVSFMHWYQQKSGT | 186 |
| | | SPKRWIYDTSKLASGVPARFSGSGSGTSYSLTISNMEAEDA | |
| | | ATYYCQQWNSDPWTFGGGSKL | |
| | CDRL1 | AGTGCCAGCTCAAGTGTAAGTTTCATGCAC | 187 |
| | | SASSSVSFMH | 188 |
| | CDRL2 | GACACATCCAAACTGGCTTCT | 189 |
| | | DTSKLAS | 190 |
| | CDRL3 | CAGCAGTGGAATAGTGACCCGTGGACG | 191 |
| | | QQWNSDPWT | 192 |

AB2085

| | | | |
|---|---|---|---|
| VH | Full | ATGGAATGGCTGTGGAACTTGCTATTTCTCATGGCAGCAGCT | 193 |
| | Length | CAAAGTATCCAAGCACAGATCCAGTTGGTGCAGTCTGGACC | |
| | (leader | TGAGCTGAAGAAGCCTGGAGAGACAGTCAGGATCTCCTG | |
| | sequence | CAAGGCTTCTGGGTATTCCTTCACAACTGCTGGAATGC | |
| | in italics) | AGTGGGTGCAAGAGATGCCAGGAAAGGGTTTGAAGTGG | |
| | | ATTGGCTGGATAGACACCCACTCTGCAGTGCCAAAATA | |
| | | TGCAGAAGACTTCAAGGGACGGTTTGCCTTCTCTTTGGA | |
| | | AACCTCTGCCAGCACTGCATATTTACAGATAAGCAACCTC | |
| | | AAAAATGAGGACACGGCTGCGTATTTCTGTGCGAGATCT | |
| | | CAGGCCGACTATGGTAACTACGGATTTGATTACTGGG | |
| | | GCCAAGGGACTCTGGTCACTGTGTCTGCA | |
| | | MEWLWNLLFLMAAAQSIQAQIQLVQSGPELKKPGETVRISCK | 194 |
| | | ASGYSFTTAGMQWVQEMPGKGLKWIGWIDTHSAVPKYA | |
| | | EDFKGRFAFSLETSASTAYLQISNLKNEDTAAYFCARSQAD | |
| | | YGNYGFDYWGQGTLVTVSA | |
| | CDRH1 | GGGTATTCCTTCACAACTGCTGGAATGCAG | 195 |
| | | GYSFTTAGMQ | 196 |
| | CDRH2 | TGGATAGACACCCACTCTGCAGTGCCAAAATATGCAGAA | 197 |
| | | GACTTCAAGGGA | |
| | | WIDTHSAVPKYAEDFKG | 198 |
| | CDRH3 | TCTCAGGCCGACTATGGTAACTACGGATTTGATTAC | 199 |
| | | SQADYGNYGFDY | 200 |

### Example 4

### Cluster Formation Studies

As DDR1 and its binding partners mediate interactions between cells, the anti-DDR1 monoclonal antibodies were analyzed for their abilities to inhibit or reduce the formation of multi-cell clusters of A431 tumor cells. In the study, about 7,000 A431 cells from human epidermoid carcinoma cell line were plated as a single-cell suspension into a 96-well plate containing 34 μL, of collagen/matrigel mixture (1 part matrigel, 2 parts collagen, 2 parts pH-balanced tissue culture media with fetal bovine serum). The cells were added with the anti-DDR1 monoclonal antibodies or the anti-DDR1 polyclonal antibody (R&D systems), or vehicle were added and incubated for 40-48 hours. Visual observation detected that the cells treated with vehicle (i.e. negative control) formed large multi-cell clusters and the cells treated with the anti-DDR1 antibodies exhibited reduced cluster formation with smaller cluster sizes.

To quantify the cluster formation, the cells were fixed in paraformaldehyde and stained with Hoechst 33342 dye. The staining images were collected using an automated fluorescent microscope (ImageXpress Micro, Molecular Devices) at 2× magnification. Images were analyzed with the MetaXpress software (Molecular Devices) as follows: the cell clusters above a specified size (approximately 20 cells) were identified and defined as large clusters, and the cells not classified as large clusters was calculated for a function of antibody dose. EC₅₀ values were determined by sigmoidal curve fitting the results of multiple averaged assay results. The EC₅₀ values and the standard deviations (StDev) are summarized in Table 5. The results of the study showed that, except AB2040, 2054, 2063, 2073, and AB2079, all tested anti-DDR1 monoclonal antibodies inhibited the cluster formation of A431 cells.

TABLE 5
EC₅₀ value of Anti-DDR1 Antibodies in Cluster Formation Assay

| Antibody | EC₅₀ (nM) | StDev |
|---|---|---|
| AB2002 | 9.72 | 3.93 |
| AB2003 | 2.38 | 0.97 |
| AB2004 | 2.28 | 1.37 |
| AB2009 | 0.43 | 0.23 |
| AB2010 | 4.84 | 2.30 |
| AB2012 | 5.41 | 2.10 |
| AB2021 | 18.51 | 6.69 |
| AB2031 | 0.63 | 0.38 |
| AB2032 | 6.89 | 4.76 |
| AB2034 | 7.87 | 3.63 |
| AB2039 | 0.93 | 2.83 |
| AB2040 | Not inhibitory | Not inhibitory |
| AB2041 | 2.03 | 2.73 |
| AB2054 | Not inhibitory | Not inhibitory |
| AB2061 | 0.88 | 0.56 |
| AB2063 | Not inhibitory | Not inhibitory |
| AB2073 | Not inhibitory | Not inhibitory |
| AB2078 | 1.82 | 1.90 |
| AB2079 | Not inhibitory | Not inhibitory |
| AB2080 | 1.24 | 0.62 |
| AB2092 | 4.18 | 1.76 |

The quantitative assessment of the normalized percent inhibition (NPI) of the cluster formation was determined using the point-test assays. The cells were treated with antibodies at a single concentration of 66 nm. Signals from the vehicle-treated cells (i.e. negative control) were defined as 0% inhibition and signals from the cells treated with the anti-DDR1 polyclonal antibody (i.e. positive control) were defined as 100% inhibition. Where quantitative data was not available, qualitative assessment of cluster formation based on visual observation was used to provide either full inhibition (i.e. equivalent to 100% NPI) for no clusters or no inhibition (i.e. equivalent to 0% NPI) for the clusters. When less than 40% NPI was detected in the cells treated an antibody in a point assay of 66 nM, there was no inhibition of cluster formation in the cells treated with the same antibody in a full dose-response curve. This indicates that the NPI values less than 40% in the point test were likely a background reading.

As shown in Table 6, more than 80% NPI was detected in most anti-DDR1 monoclonal antibodies tested, indicating that most anti-DDR1 antibodies inhibited the cluster formation in this study. Consistent with the EC₅₀ results in Table 5, the quantitative NPI values in Table 6 also indicated that the presence of AB2040, AB2054, AB2063, AB2073 and AB2079 did not inhibit the cluster formation in their treated cells.

TABLE 6
Summary of Percent Inhibition

| Antibody | NPI |
|---|---|
| AB2002 | 102.0 |
| AB2003 | 128.5 |
| AB2004 | 109.5 |
| AB2009 | 126.6 |
| AB2010 | 107.8 |
| AB2012 | 116.3 |
| AB2019 | 112.6 |
| AB2021 | 106.3 |
| AB2025 | −14.7 |
| AB2026 | 115.2 |
| AB2029 | 100.2 |
| AB2031 | 113.2 |
| AB2032 | 102.7 |
| AB2034 | 118.8 |
| AB2039 | 103.4 |
| AB2040 | 3.6 |
| AB2049 | 126.2 |
| AB2054 | 3.6 |
| AB2061 | 126.1 |
| AB2063 | −8.6 |
| AB2065 | 15.8 |
| AB2073 | −0.2 |
| AB2074 | 112.1 |
| AB2078 | 123.0 |
| AB2079 | 0.5 |
| AB2080 | Full inhibition |
| AB2085 | Full inhibition |
| AB2092 | Full inhibition |
| AB2154 | 110.9 |
| AB2163 | 107.0 |
| AB2049 | 126.2 |

### Example 5

### Localization Assay

In tumor cells grown on plastic dishes without stimulation, DDR1 is primarily localized to the outer cell membrane. Upon collagen stimulation, the localization of DDR1 is altered. To analyze the collagen-mediated relocalization of DDR1 in the presence of anti-DDR1 antibodies, HCT-116 tumor cells (human colorectal carcinoma, ATCC) stably over-expressing DDR1 were used. About 5,000 cells were plated in 96 well plate and cultured in serum-free media containing 40 μg/mL collagen I. A titration of anti-DDR1 monoclonal antibodies were added to the cells. After 16 hours, the cells were fixed in paraformaldehyde and stained with a 1:1600 dilution of the rabbit anti-DDR1 antibody (Cell Signaling Technology cat#5583) followed by an Alexa-647 conjugated anti-rabbit antibody. Cells were also counterstained with Hoechst 33342 and Whole Cell Green dye.

Stained imagines were collected using an automated fluorescent microscope (ImageXpress Micro, Molecular Devices) at 10× magnification. The intensity of the Alexa-647 in the area outside the cells and that of inside the cells was quantified using the MetaXpress software (Molecular Devices). The intensity of the pixels was plotted as a function of antibody concentration, and EC₅₀ values were determined by sigmoidal curve fitting. AB0039, AB2004 and AB2041 inhibited the relocation of DDR1 that was normally stimulated by collagen at EC₅₀ value of 0.45 nM (standard deviation or s.d. 0.36), 1.4 nM (s.d. 0.92), and 2.2 nM (s.d. 1.29), respectively. AB2092 and AB2078 did not alter the relocalization of DDR1 that was stimulated by collagen.

### Example 6

### NFκB Reporter and Phosphorylation Studies

Anti-DDR1 antibodies were also characterized using a cell-based NFκB luciferase reporter assay and two phosphorylation assays were used. In the NFκB reporter assay, HCT-116 tumor cells (human colorectal carcinoma line, ATCC) stably transfected to over-express DDR1 were plated in complete medium (DMEM/10% FBS) at 8×10⁶ cells per 10 cm plate. When cells reached 90% confluence, they were transfected with 20 μg NFκB-Luc and 2 μg Renilla-Luc plasmids using Lipofectamine 2000 in serum-free Opti-MEM medium (Life Technologies). Four hours post-transfection, medium was exchanged for complete medium and cells were incubated overnight. The next day, cells were detached from the plate with Accutase (Life Technologies) and re-plated into 96-well white-bottom plates at 1×10⁵ cells per well in the presence or absence of anti-DDR1 antibody at various concentrations, in triplicate. After 30-60 minutes, cells were stimulated with 50 μg/ml Collagen II (DiscoveRx) or 50 μM acetic acid vehicle. After 20-24 hours, cells were analyzed for luciferase activity using the Dual-Glo luciferase assay system (Promega).

For data analysis, the raw firefly luciferase values were divided by the raw Renilla luciferase values in the same well (FF values/Ren values) to normalize for transfection efficiency and the resulting ratio was multiplied by a scaling factor to achieve whole numbers (scaling factor=100). Signals from the cells treated with collagen alone (i.e. positive control) were defined as 100%, and signals from the cells treated without collagen (i.e. negative control) were defined as 0%. All signals or values for the cells treated with anti-DDR1 monoclonal antibodies were normalized by subtracting the background average then dividing by the average value of the positive control. The normalized values were plotted as a function of antibody concentration, and EC₅₀ values were determined by sigmoidal curve fitting (Prism software, GraphPad). A summary of EC₅₀ and EC %) values is provided in Table 7. The results provided are the average of two or more experiments. Lower EC₅₀ value indicates that the anti-DDR1 antibody is more potent and inhibits the DDR1 protein at lower concentration.

TABLE 7
Summary of EC₅₀ and EC₉₀ values

| Antibody | EC₅₀ (nM) | Stand. Dev. | EC₉₀ (nM) |
|---|---|---|---|
| AB2004* | 3.70 | 2.36 | 6.95 |
| AB2039* | 1.63 | 1.00 | 4.61 |
| AB2041 | 1.12 | 0.37 | 4.17 |
| AB2078* | 3.01 | 1.64 | 6.66 |
| AB2092 | 1.13 | 1.07 | 4.73 |

*The values were an average of multiple antibody clones that had the same sequence (AB2004 = AB2004 and AB2003; AB2039 = AB2009, AB2026, AB2031, and AB2039; AB2078 = AB2061, AB2074, and AB2078).

In the first phosphorylation studies, a cell-based assay from DiscoveRx Corporation was used to measure the inhibition of phosphorylation of DDR1 at tyrosine 513 in response to collagen stimulation. In this study, engineered U20S cells (human osteosarcoma cell line) that express DDR1 with a ProLink™ Tag (PK) and enzyme acceptor (EA) tagged-SHC1 adaptor protein were used. Cells were plated at 20,000 cells/well in 96 plates using DiscoverRx Plating Media 16. After 30 minutes, antibodies in serial dilution by phosphate buffered saline (PBS) was added to the cells. The cells were then treated with Collagen II at 12.5 μg/mL for 24 hours to initiate DDR1 phosphorylation and recruitment of SHC-1-EA to the receptor. This interaction leads to complementation of the two β-galactosidase enzyme fragments (EA and PK) to make active enzyme, which upon addition of DiscoveRx substrate solution hydrolyzes the substrate to generate chemiluminescent signal proportional to the tyrosine 513 DDR1 phosphorylation. Chemiluminescence was measured using a BioTek Synergy plate reader and the data were plotted as relative light units versus antibody concentration. EC₅₀ values were calculated using 4-parameter non-linear fit using Prism software (GraphPad). Mean EC₅₀ values are shown below in Table 8. Some of the results are averaged value from multiple independent experiments.

TABLE 8
Potency of Anti-DDR1 Antibodies
Measured by a Phosphorylation Assay

| | Mean EC₅₀ |
|---|---|
| Antibody | (nM) |
| AB2002 | 4.5 |
| AB2003 | 3 |
| AB2004 | 1.85 |
| AB2009 | 1.5 |
| AB2010 | 7.6 |
| AB2012 | 8.6 |
| AB2019 | 6.6 |
| AB2021 | 7.71 |
| AB2025 | No inhibition |
| AB2026 | 1.7 |
| AB2029 | 4.9 |
| AB2031 | 0.99 |
| AB2032 | 7.72 |
| AB2034 | 5.2 |
| AB2039 | 1.1 |
| AB2040 | No inhibition |
| AB2041 | 2.65 |
| AB2049 | 4.68 |
| AB2054 | No inhibition |
| AB2061 | 2.5 |
| AB2063 | No inhibition |
| AB2065 | 25.7 |
| AB2073 | No inhibition |
| AB2074 | 1.5 |
| AB2078 | 1.33 |
| AB2079 | No inhibition |
| AB2080 | 2.2 |
| AB2085 | 3.9 |
| AB2092 | 2.45 |
| AB2154 | 24.2 |
| AB2163 | 14.5 |

In the second phosphorylation assay, the ability of anti-DDR1 antibodies to inhibit DDR1 kinase activation in response to collagen stimulation was evaluated using an ELISA assay to detect phospho-tyrosine-DDR1 protein in T47D cells (human ductal breast epithelial tumor cell line).

For this study, T47D cells were seeded in a 96-well plate at 5×10⁵ cells per well in RPMI-10% FBS overnight at 37° C. The following day, the medium was replaced with cold serum-free RPMI containing 20 μg/mL collagen (BD Biosciences) and added the anti-DDR1 antibodies at different concentrations. Cells incubated with no collagen (i.e. media alone) and collagen-only were used as controls. After overnight treatment, cell lysates were prepared by removing conditioned medium and suspended in 200 μL/well of 1× lysis buffer (Cell Signaling Technology) containing protease inhibitors (Sigma). Samples were incubated for 15 minutes at 4° C. and centrifuged for 10 minutes at 4° C. at 1000×g. Clarified cell lysates were then added to a 96 well plate pre-coated with the anti-DDR1 capture antibody (Cell Signaling Technologies) and incubated overnight at 4° C. The next day, anti-phospho-tyrosine HRP conjugated detection antibody (R&D Systems) was added for 2 hours at room temperature, followed by HRP substrate (R&D Systems). After blue color developed, the reaction was stopped with addition of 2N sulfuric acid and measured for absorbance at 450 nm using a spectrophotometer within 30 minutes.

The absorbance values were plotted as a function of antibody concentration, and EC₅₀ values were determined by sigmoidal curve fitting using Prism software (GraphPad). Results (in some cases shown as average values) were summarized in Table 9. The cells treated with certain antibodies, including AB2025 and AB2063, did not exhibit any inhibition even at highest concentration. This suggests that they are non-inhibitory antibodies. Also, some antibodies, including AB2040, AB2054, AB2073 and AB2079, exhibited inhibition that was non-maximal, suggesting that they are incomplete inhibitory antibodies. All other anti-DDR1 antibodies tested, at various concentration, exhibited 100% inhibition.

TABLE 9
Inhibition of DDR1 Kinase Activation by Anti-DDR1
Antibodies Measured by a DDR1 Phospho-specific ELISA

| | | Maximal |
|---|---|---|
| Antibody | EC₅₀ (nM) | inhibition |
| AB2002 | 1.07 | 100% |
| AB2003 | 0.42 | 100% |
| AB2004 | 0.43 | 100% |
| AB2009 | 0.47 | 100% |
| AB2010 | 0.63 | 100% |
| AB2012 | 0.62 | 100% |
| AB2019 | 0.66 | 100% |
| AB2021 | 2.26 | 100% |
| AB2025 | No inhibition | n/a |
| AB2026 | 0.39 | 100% |
| AB2029 | 0.55 | 100% |
| AB2031 | 0.40 | 100% |
| AB2032 | 0.64 | 100% |
| AB2034 | 0.63 | 100% |
| AB2039 | 0.43 | 100% |
| AB2040 | 0.34 | Non-maximal |
| AB2041 | 0.72 | 100% |
| AB2049 | 0.96 | 100% |
| AB2054 | 0.36 | Non-maximal |
| AB2061 | 0.66 | 100% |
| AB2063 | No inhibition | n/a |
| AB2065 | 0.35 | 100% |
| AB2073 | 0.45 | Non-maximal |
| AB2074 | 0.47 | 100% |
| AB2078 | 0.39 | 100% |
| AB2079 | 0.13 | Non-maximal |
| AB2080 | 4.67 | 100% |
| AB2085 | 0.51 | 100% |
| AB2092 | 0.52 | 100% |
| AB2154 | 3.23 | 100% |
| AB2163 | 4.00 | 100% |

### Example 7

### ELISA Assay

In this study, ELISA was used to determine the affinity of murine anti-DDR1 monoclonal antibodies to the extracellular domain (ECD) of either human or murine DDR1. A 96-well MaxiSorb ELISA plates (Nunc) were coated with 2 μg/mL of one of three purified recombinant tagged proteins: human ECD-DDR1-His protein, human ECD-DDR1-Fc, or mouse ECD-DDR1-Fc overnight at 4° C. The next day, the plates were blocked with 5% bovine serum albumin (BSA, Jackson Immunoresearch) prior to the addition of serially diluted anti-DDR1 monoclonal antibodies. Antibodies were incubated for 1 hr at room temperature. The binding of antibody to the antigen was detected by incubating samples with anti-mouse IgG-HRP (horse radish peroxidase) secondary detection antibody (Jackson Immunoresearch) for 1 hr. After a washing step, 3,3′,5,5″-tetramethylbenzidine (TMB) reagent was added for HRP to produce a colorimetric signal. The reaction was stopped by addition of 1M hydrochloric acid after 2 minutes and measured for absorbance (optical density, OD) at 450λ on a Molecular Devices plate reader. The OD values were plotted against antibody concentration and fitted using a using 4-parameter logistic equation in SoftMax software (Molecular Devices). The apparent affinity constant (K_{D}) was taken from the C-parameter of the equation (y=(A−D)/(1+(x/C)̂B)+D). The apparent affinity constants were summarized (in some cases shown as average values) in Table 10 below. Lower value indicates higher binding affinity of the antibody to the antigen.

TABLE 10
Affinity of Murine Anti-DDR1 Monoclonal Antibodies
for Human DDR1 Measured by ELISA

| | Average K_{D} | | |
|---|---|---|---|
| | vs. human | human | mouse |
| | ECD-DDR1- | ECD-DDR1-Fc | ECD-DDR1-Fc |
| AB # | His (nM) | (Kd in nM) | (Kd, in nM) |
| AB2002 | 0.233 | 0.268 | NB |
| AB2003 | 0.153 | 0.216 | NB |
| AB2004 | 0.181 | 0.154 | NB |
| AB2009 | 0.197 | 0.257 | 6.93 |
| AB2010 | 0.398 | 0.431 | 33.7 |
| AB2012 | 0.534 | 0.509 | 60.2 |
| AB2019 | 0.372 | 0.324 | NB |
| AB2021 | 0.153 | 0.252 | NB |
| AB2025 | 0.207 | 0.231 | 0.241 |
| AB2026 | 0.161 | 0.298 | 17.9 |
| AB2029 | 0.397 | 0.302 | NB |
| AB2031 | 0.148 | 0.257 | 17.3 |
| AB2032 | 0.5 | 0.596 | NB |
| AB2034 | 0.311 | 0.256 | NB |
| AB2039 | 0.205 | 0.346 | 2.37 |
| AB2040 | 0.115 | 0.254 | NB |
| AB2041 | 0.617 | 0.561 | NB |
| AB2049 | 0.576 | 0.362 | NB |
| AB2054 | 0.154 | 0.31 | NB |
| AB2061 | 0.391 | 0.418 | 28 |
| AB2063 | 0.387 | 0.344 | 0.891 |
| AB2065 | 0.197 | 0.234 | NB |
| AB2073 | 0.2 | 0.209 | NB |
| AB2074 | 0.47 | 0.259 | 28.5 |
| AB2078 | 0.554 | 0.374 | 10.9 |
| AB2079 | 0.07 | 0.0883 | NB |
| AB2080 | 0.418 | 0.669 | 2.89 |
| AB2085 | 0.267 | 0.23 | NB |
| AB2092 | 0.275 | 0.287 | 1.19 |

n/a: not available
NB: no binding

### Example 8

### Interaction Between DDR1Protein Residues and Anti-DDR1Antibodies

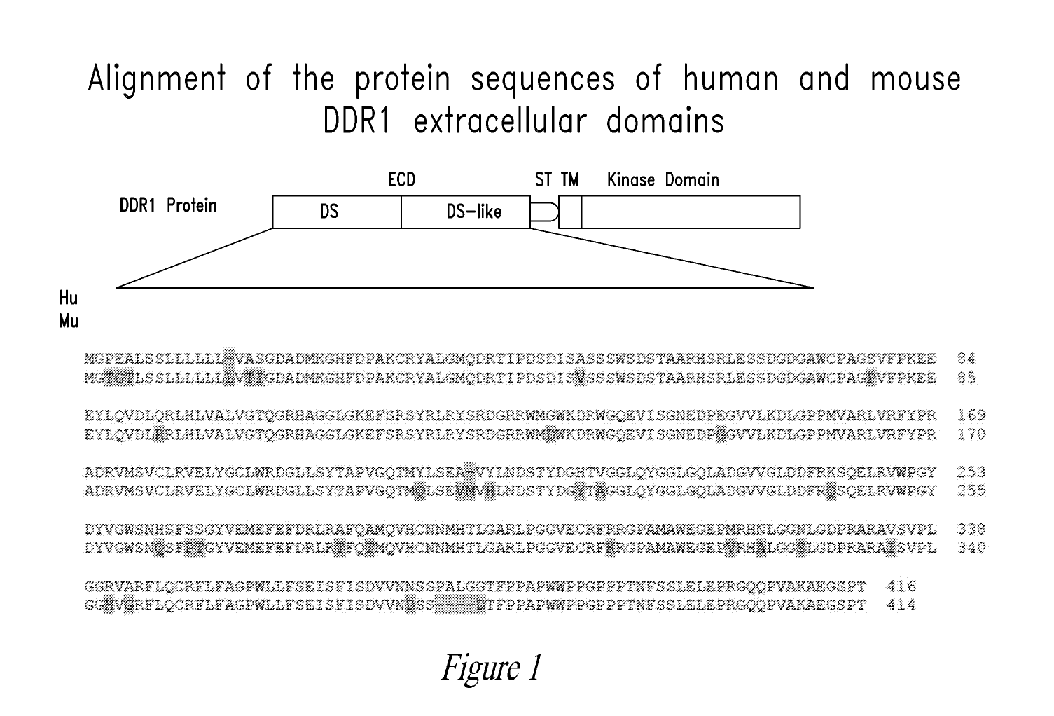

To map out the residues on the DDR1 extracellular domain (ECD) that interact with anti-DDR1 monoclonal antibodies, a series of DDR1 point mutations were generated. The immunoassays were used to determine the strength of antibody-antigen interaction. First, the ECD from mouse DDR1 was subcloned from a cDNA-containing plasmid (Origene #MG223468) into both a His-tag expression vector (pSectag2-hygro, Life Technologies) and a human IgG1-Fc expression vector (pFuse-hIgG1-Fc1, Invivogen), followed by standard transformation protocol in One shot TOP 10 chemically competent cells (Life Technologies, Cat:C4040-06). The alignment of the protein sequences of human and mouse DDR1ECD domains and the identification of non-conserved residues between the two species is shown in FIG. 1. A series of individual “humanized” point mutations were introduced into the mouse DDR1ECD expression plasmids described above, using the QuickChange II Site-Directed Mutagenesis kit (Agilent Technologies). Similarly, human DDR1ECD was subcloned from human DDR1 cDNA (Origene) into the human IgG1-Fc expression vector described above, and individual “murinized” point mutations were introduced using the QuickChange II kit as before. All resulting plasmids were introduced individually into HEK 293 cells for protein expression using a standard transfection protocol with Lipofectamine 2000 (Life Technologies).

To determine antibody binding, mouse or human ECD-DDR1 proteins were first purified by either nickel affinity chromatography or protein A chromatography then used in an ELISA assay. MaxiSorp 96-well plates (Nunc) were coated over night with 1 μg/mL of purified DDR1-ECD-His or -Fc proteins at 100 μL/well in PBS, then blocked overnight with 5% (w/v) bovine serum albumin (BSA) in PBS at a volume of 200 μL/well. After washing the plate 3 times in PBS+0.05% Tween-20 (PBS-T), 0.1-1 pg/mL antibody was added to the plate in duplicate at 100 μl/well and incubated on the plate rocker for 1 hour at room temperature. After another series of washes, goat anti-Mouse IgG-HRP (ThermoScientific) was added to the plate at 1:10,000 dilution in 0.5% BSA/PBS at 100 μL/well, followed by incubation on the plate rocker for 1 hour and repeated washes as described above. Finally, plates were developed by addition of TMB substrate at 100 μL/well; the reaction was stopped after 30 seconds with additional of 100 μL/well of 1M HCl. Absorbance at 450λ was measured on a SpectramaxM5 plate reader (Molecular Devices).

For each anti-DDR1 antibody, residues involved in receptor binding were identified by comparing the ELISA signal intensity for binding to wild-type mouse or human DDR1-ECD protein, compared to each of the mutant proteins. For antibodies with higher affinity to human than mouse DDR1, residues were considered part of the epitope if humanization of the mouse residue resulted in increased binding to mouse DDR1-ECD, and/or the murinization of the human residue resulted in decreased binding to human DDR1-ECD.

For antibodies with higher affinity for human DDR1 than for mouse DDR1, residues in the DDR1 ECD identified as involved in binding of anti-DDR1 antibodies are as follows. “Humanizing” mutations that increase binding to mouse DDR1-ECD include: AB2009 (Q245K), AB2010 (Q245K, Y220H, K308R), AB2026 (Q245K), AB2031 (Q245K), AB2039 (Q245K), AB2061 (Q245K), AB2074 (Q245K), AB2078 (Q245K), AB2080 (G149E), AB2092 (Q245K), AB2154 (H343R), and AB2163 (H343R). “Murinizing” mutations, or other mutations, that decrease binding to human DDR1-ECD include AB2039 (D239A, R242A, R242T, K243Q, S244T). In addition, studies of these mutations in three-dimensional model of human DDR1-ECD protein to illustrate the interaction or binding between the protein residue and anti-DDR1 antibodies. The results of this study were summarized in Table 11.

TABLE 11

| Human | |
|---|---|
| DDR1-ECD | |
| Residue/ | |
| amino acid | Anti-DDR1 Antibodies |
| S78 | AB2034, AB2085 |
| E148 | AB2002, AB2080 |
| S131 | AB2041, AB2049 |
| Y203 | AB2065 |
| H218 | AB2010, AB2019, AB2032, AB2029, AB2041, AB2049 |
| V220 | AB2041, AB2049 |
| K243 | AB2010, AB2032, AB2003, AB2009, AB2026, AB2031, |
| | AB2039, AB2021, AB2061, AB2074, AB2078, AB2092 |
| R306 | AB2010, AB2019, AB2032, AB2041, AB2049 |
| R341 | AB2154, AB2163 |

For antibodies displaying no detectable binding to wild-type mouse DDR1-ECD, residues in the DDR1 ECD identified as involved in binding of anti-DDR1 antibodies are as follows. “Humanizing” mutations that cause initiation of binding to mouse DDR1-ECD include: AB2002 (G149E), AB2003 (Q245K), AB2021 (Q245K), AB2029 (Y220H), AB2032 (Q245K, K308R, Y220H), AB2034 (P79S), AB2041 (Y220H, D132G, K308R, A222V), AB2049 (Y220H, D132G, K308R, A222V), AB2065 (Q204Y), AB2085 (P79S), and AB2019 (K308R, Y220H). “Murinizing” mutations, or other mutations, that decrease binding to human DDR1-ECD include AB2041 (H218Y, V220A, Y225T, R306K).

### Example 9

### Epitope Binning of Anti-DDR1Monoclonal Antibodies by Measurement of Cross-Blocking

Monoclonal antibodies that bind to the extracellular domain (ECD) of human DDR1 protein were grouped into epitope bins by assessing which pairs of antibodies were able to bind simultaneously to the ECD, and/or which antibodies cross-blocked each other. Cross-blocking indicates that the two antibodies may bind overlapping epitopes on the target protein.

Epitope binning experiments were performed on an OctetRed384 (ForteBio). Data were collected at 30° C. in PBS (pH7.4) supplemented with Tween (0.005%) and BSA (0.01%), unless stated otherwise. Purified recombinant huDDR1-ECD-His and huDDR1-Fc were prepared from HEK293 cell conditioned medium and purified as described infra. Antibodies were expressed either from mouse hybridoma cells, or from transient heavy and light chain DNA transfection in HEK293 cells, and purified via protein A chromatography. To perform epitope binning experiments on the OctetRed, anti-mouse-Fc coated biosensors (AMC) or anti-human-Fc coated biosensors (AHC) were used to capture the first mouse (20-200 ng/ml) or human (20-200 ng/ml) anti-DDR1 antibodies. Biosensors were blocked with either a non-specific mouse or human IgG (2 μM). Antibody-coated biosensors were then dipped into wells containing a huDDR1 antigen (100 nM, 200 s) followed by an array of second anti DDR1 antibodies. Under this experimental configuration, using a pair of antibodies that favor complete blocking, no binding response will be detected after the addition of second antibody. This binary analysis was used to assign antibodies to epitope bins according to their blocking profiles relative to one another. The summary of epitope bins is shown in Table 12. Antibodies in the same epitope bin cross-block each other, i.e., they are not capable of simultaneous pairwise binding to DDR1 ECD. Antibodies that cross-blocked with more than one epitope bin are listed as “mixed”.

TABLE 12

| | | | | | | Mixed |
|---|---|---|---|---|---|---|
| Epitope | Epitope | Epitope | Epitope | Epitope | Epitope | epitope |
| bin #1 | bin #2 | bin #3 | bin #4 | bin #5 | bin #6 | bins |
| AB2010 | AB2040 | AB2003 | AB2002 | AB2034 | AB2154 | AB2025 |
| AB2019 | AB2054 | AB2004 | AB2080 | AB2085 | AB2163 | AB2063 |
| AB2029 | AB2065 | AB2009 | | ADI-9695 | | ADI-9669 |
| AB2032 | AB2073 | AB2012 | | ADI-9697 | | ADI-9671 |
| AB2041 | AB2079 | AB2021 | | ADI-9705 | | ADI-9684 |
| AB2049 | | AB2026 | | | | ADI-9689 |
| ADI-9677 | | AB2031 | | | | |
| ADI-9682 | | AB2039 | | | | |
| | | AB2061 | | | | |
| | | AB2074 | | | | |
| | | AB2078 | | | | |
| | | AB2092 | | | | |

Throughout this application, various publications, patent applications and patents are referenced. The disclosures of each of these references are hereby incorporated by reference herein in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

1. An isolated antibody, comprising one or more heavy chain complementarity determining region (CDRH), wherein the CDRH is selected from the group consisting of:
a) the heavy chain variable (VH) region comprising the amino acid sequence set forth as SEQ ID NO: 4;
b) the VH region comprising the amino acid sequence set forth as SEQ ID NO: 20;
c) the VH region comprising the amino acid sequence set forth as SEQ ID NO: 36;
d) the VH region comprising the amino acid sequence set forth as SEQ ID NO: 52;
e) the VH region comprising the amino acid sequence set forth as SEQ ID NO: 68;
f) the VH region comprising the amino acid sequence set forth as SEQ ID NO: 84;
g) the VH region comprising the amino acid sequence set forth as SEQ ID NO: 100;
h) the VH region comprising the amino acid sequence set forth as SEQ ID NO:116;
i) the VH region comprising the amino acid sequence set forth as SEQ ID NO:132;
I) the VH region comprising the amino acid sequence set forth as SEQ ID NO:148;
k) the VH region comprising the amino acid sequence set forth as SEQ ID NO:156;
l) the VH region comprising the amino acid sequence set forth as SEQ ID NO:164;
m) the VH region comprising the amino acid sequence set forth as SEQ ID NO:178;
n) the VH region comprising the amino acid sequence set forth as SEQ ID NO:194;
o) the VH region comprising the amino acid sequence set forth as SEQ ID NO:203;
p) the VH region comprising the amino acid sequence set forth as SEQ ID NO:204;
q) the VH region comprising the amino acid sequence set forth as SEQ ID NO:205;
r) the VH region comprising the amino acid sequence set forth as SEQ ID NO:206;
s) the VH region comprising the amino acid sequence set forth as SEQ ID NO:227;
t) the VH region comprising the amino acid sequence set forth as SEQ ID NO:228;
u) the VH region comprising the amino acid sequence set forth as SEQ ID NO:229;
v) the VH region comprising the amino acid sequence set forth as SEQ ID NO:230;
w) the VH region comprising the amino acid sequence set forth as SEQ ID NO:231; and
x) the VH region comprising the amino acid sequence set forth as SEQ ID NO:232.

2. The isolated antibody of claim 1, wherein the one or more CDRH comprises a CDRH3.

3. The isolated antibody of claim 2, wherein the CDRH3 comprises the amino acid sequence set forth as SEQ ID NO: 10, 26, 42, 58, 74, 90, 106, 122, 138, 154, 162, 170, 184, or 200.

4. The isolated antibody of any of claims 1-3, wherein the one or more CDRH comprises a CDRH1 and a CDRH2.

5. The isolated antibody of claim 4, wherein the CDRH1 comprises the amino acid sequence set forth as SEQ ID NO: 6, 22, 38, 54, 70, 86, 102, 118, 134, 150, 158, 166, 180, or 196, and the CDRH2 comprises the amino acid sequence set forth as SEQ ID NO: 8, 24, 40, 56, 72, 88, 104, 120, 136, 152, 160, 168, 182, or 198.

6. The isolated antibody of any of claims 1-5, wherein the antibody comprises a VH region comprising the amino acid sequence set forth in SEQ ID NO: 4, 20, 36, 52, 68, 84, 100, 116, 132, 148, 156, 164, 178, or 194 with or without the leader sequence.

7. The isolated antibody of any of claims 1-5, wherein the antibody comprises a VH region comprising the amino acid sequence set forth in SEQ ID NO: 203, 204, 205, 206, 227, 228, 229, 230, 231, or 232.

8. The isolated antibody of any of claims 1-7, wherein the antibody further comprises one or more light chain complementarity determining region (CDRL), wherein the CDRL is selected from the group consisting of:
a) the light chain variable (VL) region comprising the amino acid sequence set forth as SEQ ID NO: 12;
b) the VL region comprising the amino acid sequence set forth as SEQ ID NO: 28;
c) the VL region comprising the amino acid sequence set forth as SEQ ID NO: 44;
d) the VL region comprising the amino acid sequence set forth as SEQ ID NO: 60;
e) the VL region comprising the amino acid sequence set forth as SEQ ID NO: 76;
f) the VL region comprising the amino acid sequence set forth as SEQ ID NO: 92;
g) the VL region comprising the amino acid sequence set forth as SEQ ID NO:108;
h) the VL region comprising the amino acid sequence set forth as SEQ ID NO:124;
i) the VL region comprising the amino acid sequence set forth as SEQ ID NO:140;
j) the VL region comprising the amino acid sequence set forth as SEQ ID NO:186;
k) the VL region comprising the amino acid sequence set forth as SEQ ID NO:207;
l) the VL region comprising the amino acid sequence set forth as SEQ ID NO:208;
m) the VL region comprising the amino acid sequence set forth as SEQ ID NO: 209;
n) the VL region comprising the amino acid sequence set forth as SEQ ID NO:210;
o) the VL region comprising the amino acid sequence set forth as SEQ ID NO:212;
p) the VL region comprising the amino acid sequence set forth as SEQ ID NO:220;
q) the VL region comprising the amino acid sequence set forth as SEQ ID NO:233;
r) the VL region comprising the amino acid sequence set forth as SEQ ID NO:234;
s) the VL region comprising the amino acid sequence set forth as SEQ ID NO:235;
t) the VL region comprising the amino acid sequence set forth as SEQ ID NO:236; and
u) the VL region comprising the amino acid sequence set forth as SEQ ID NO:237;.

9. The isolated antibody of claim 8, wherein the one or more CDRL comprises a CDRL3.

10. The isolated antibody of claim 9, wherein the CDRL3 comprises the amino acid sequence set forth as SEQ ID NO: 18, 34, 50, 66, 82, 98, 114, 130, 146, 176, 192, 218, or 226.

11. The isolated antibody of any of claims 8-10, wherein the one or more CDRL comprises a CDRL1 and a CDRL2.

12. The isolated antibody of claim 11, wherein the CDRL1 comprises the amino acid sequence set forth as SEQ ID NO: 14, 30, 46, 62, 78, 94, 110, 126, 142, 172, 188, 214, or 222, and the CDRL2 comprises the amino acid sequence set forth as SEQ ID NO: 16, 32, 48, 64, 80, 96, 112, 128, 144, 174, 190, 216, or 224.

13. The isolated antibody of any of claims 1-12, wherein the antibody comprises a VL region comprising the amino acid sequence set forth in SEQ ID NO: 12, 28, 44, 60, 76, 92, 108, 124, 140, 186, 212, or 220, with or without the leader sequence.

14. The isolated antibody of any of claims 1-12, wherein the antibody comprises a VL region comprising the amino acid sequence set forth in SEQ ID NO: 207, 208, 209, 210, 233, 234, 235, 236, or 237.

15. The isolated antibody of any of claims 1-14, wherein the antibody comprises:
a) a VH region comprising the amino acid sequence set forth as SEQ ID NO: 4, with or without the leader sequence and a VL region having the amino acid sequence set forth as SEQ ID NO: 12, with or without the leader sequence;
b) a VH region comprising the amino acid sequence set forth as SEQ ID NO: 20, with or without the leader sequence and a VL region having the amino acid sequence set forth as SEQ ID NO: 28, with or without the leader sequence;
c) a VH region comprising the amino acid sequence set forth as SEQ ID NO: 36, with or without the leader sequence and a VL region having the amino acid sequence set forth as SEQ ID NO: 44, with or without the leader sequence;
d) a VH region comprising the amino acid sequence set forth as SEQ ID NO: 52, with or without the leader sequence and a VL region having the amino acid sequence set forth as SEQ ID NO: 60, with or without the leader sequence;
e) a VH region comprising the amino acid sequence set forth as SEQ ID NO: 68, with or without the leader sequence and a VL region having the amino acid sequence set forth as SEQ ID NO: 76, with or without the leader sequence;
f) a VH region comprising the amino acid sequence set forth as SEQ ID NO: 84, with or without the leader sequence and a VL region having the amino acid sequence set forth as SEQ ID NO: 92, with or without the leader sequence;
g) a VH region comprising the amino acid sequence set forth as SEQ ID NO: 100, with or without the leader sequence and a VL region having the amino acid sequence set forth as SEQ ID NO: 108, with or without the leader sequence;
h) a VH region comprising the amino acid sequence set forth as SEQ ID NO: 116, with or without the leader sequence and a VL region having the amino acid sequence set forth as SEQ ID NO: 124, with or without the leader sequence;
i) a VH region comprising the amino acid sequence set forth as SEQ ID NO: 132, with or without the leader sequence and a VL region having the amino acid sequence set forth as SEQ ID NO: 140, with or without the leader sequence;
j) a VH region comprising the amino acid sequence set forth as SEQ ID NO: 178, with or without the leader sequence and a VL region having the amino acid sequence set forth as SEQ ID NO: 186, with or without the leader sequence;
k) a VH region comprising the amino acid sequence set forth as SEQ ID NO: 148, with or without the leader sequence and a VL region having the amino acid sequence set forth as SEQ ID NO: 212, with or without the leader sequence;
l) a VH region comprising the amino acid sequence set forth as SEQ ID NO: 156, with or without the leader sequence and a VL region having the amino acid sequence set forth as SEQ ID NO: 220, with or without the leader sequence;
m) a VH region comprising the amino acid sequence set forth in any one of SEQ ID NOs: 202-206, with or without the leader sequence and a VL region having the amino acid sequence set forth in any one of SEQ ID NOs: 207-210, with or without the leader sequence; or
n) a VH region comprising the amino acid sequence set forth in any one of SEQ ID NOs: 227-232, with or without the leader sequence and a VL region having the amino acid sequence set forth in any one of SEQ ID NOs: 233-237, with or without the leader sequence.

16. An isolated antibody that competes for binding to a DDR1 protein with the antibody of any of claims 1-15.

17. The isolated antibody of any of claims 1-16, wherein the antibody is human or humanized.

18. The isolated antibody of any of claims 1-17, wherein the antibody is an antibody fragment.

19. The isolated antibody of any of claims 1-18, wherein the antibody specifically binds to a DDR1 protein.

20. The antibody of claim 19, wherein the antibody inhibits activity of the DDR1 protein.

21. A method for treating a disease or condition associated with DDR1 in a subject, the method comprising: administering to the subject an antibody that specifically binds to DDR1.

22. The method of claim 21, wherein the antibody is the antibody of any of claims 1-20.

23. The method of claim 22, wherein the disease or condition is cancer, metastasis, tumor invasion, angiogenesis, a fibrotic disease or condition, an immunological disease or condition, or a disease associated with deregulation of extracellular matrix production.

24. A detection method, comprising detecting an expression level or activity of a DDR1 protein in a biological sample from a subject, wherein the level indicates the presence or severity of a disease or condition associated with DDR1.

25. The method of claim 24, wherein the detection is carried out using the antibody of any of claims 1-20.
